(19) United States
(12) Patent Application Publication
(10) Pub. No.: US 2016/0145643 A1
(43) Pub. Date: May 26, 2016

(54) Endothelial-targeted Adenoviral Vectors, Methods and Uses Therefor

(71) Applicant: Washington University, Saint Louis, MO (US)

(72) Inventors: Jefferey Arbeit, Saint Louis, MO (US); David Curiel, Saint Louis, MO (US)

(73) Assignee: Washington University, Saint Louis, MO (US)

(21) Appl. No.: 14/966,078

(22) Filed: Dec. 11, 2015

Related U.S. Application Data
(63) Continuation of application No. PCT/US14/42204, filed on Jun. 12, 2014.
(60) Provisional application No. 61/834,385, filed on Jun. 12, 2013.

(51) Int. Cl. C12N 15/86; C12N 7/00; C07K 14/715; A61K 48/00; C12N 9/78; C07K 14/71

(52) U.S. Cl. CPC C12N 15/86; C12N 9/78; C07K 14/71; C07K 14/7158; A61K 48/0058; C12N 7/00; C12Y 305/04001; A61K 38/00

(57) ABSTRACT

Disclosed are adenovirus vectors comprising a ROBO4 enhancer/promoter operatively linked to a transgene. Also disclosed are adenovirus vectors comprising a chimeric AD5-T4 phage fibritin shaft, a trimerization domain displaying a myeloid cell-binding peptide (MBP), and a ROBO4 enhancer promoter operatively linked to a transgene. Also disclosed are methods of expressing a transgene in an endothelial cell in vivo, comprising administering to a mammal an adenovirus comprising a ROBO4 enhancer/promoter operatively linked to a transgene. Also disclosed are uses of the adenoviral vectors, including mobilization of granulocytes, monocytes and lymphocytes from bone marrow, mobilization of cancer cells in vivo, selective targeting of endothelial cells, and cancer treatment methods.

### CROSS REFERENCE TO RELATED APPLICATION

This application claims benefit of and priority to PCT/US14/42204 filed Jun. 12, 2014. PCT/US14/42204 claims priority from U.S. Provisional Application Ser. No. 61/834,385 filed Jun. 12, 2013, each of which is incorporated herein by reference in its entirety.

### STATEMENT REGARDING FEDERALLY SPONSORED RESEARCH

This work received support from NIB R01CA159959, and CA154697. The government may have certain rights in the invention.

### INTRODUCTION

In stem cell biology, stem cells possess the properties of self-renewal, proliferative quiescence, and organ-tumor multi-lineage repopulation (Barker et al. 2010). Stem cells can require a host cellular niche to maintain their functions (Voog and Jones 2010). Stem cells in most organs and tissues persist for the organism lifetime (Voog and Jones 2010). Persistence can be due to markedly prolonged cell cycle times inferred by prolonged retention of the nucleotide analogs tritiated thymidine or bromodeoxyuridine (BrdU), or assayed by chromatin bound histone 2B fluorophore fusion proteins (Foudi et al. 2009). Tissue stem cell biology has been conceptually modeled based on the hierarchical organization of stem and progenitor cells in the hematopoietic system (Essers and Trumpp 2010). A hematopoietic cellular hierarchy has been identified and stem cells isolated by fluorescence activated cell sorting of cell surface markers combined with functional cell culture and intact animal repopulation and colony forming assays (Rieger and Schroeder 2012: Mayle et. al. 2013). Existence of prostate stem cells, in particular in the rodent, was strongly suggested by studies demonstrating 30 cycles of prostate regeneration following castration and exogenous androgen provision (English et al. 1987). A combination of flow cytometric markers, serial cell culture, and tissue recombination with kidney subcapsular grafting enabled construction of murine and human prostate stem/differentiated hierarchies (Goldstein and Witte 2012; Guo et al. 2012). Prostate stem like cells form spheroids “prostaspheres” when grown in anchorage-independent cell culture (Lukacs et al. 2010; Rhim 2013). Prostaspheres self-renew during prolonged serial passage, and repopulate tubules and ducts, forming prostate organoids when re-implanted into mice (Azuma et al, 2005: Guo et al. 2012). Stem cells have been identified in classical PCA cell lines including PC3, DUI44. parental LNCaP, and derivative LNCaP-2B cells (Miki et al. 2007).

Approaches targeting metastatic neovasculature are needed. An option can be tumor vascular endothelial cell (EC) adenoviral (Ad) vector targeting. Although EC transductional and transcriptional targeting has been accomplished, vector administration approaches of limited clinical utility, lack of tumor-wide EC expression quantification, and a failure to address avid liver sequestration, has challenged prior research. Previous vascular targeted drugs and biologics aim to destroy/inhibit the formation of new vasculature in an attempt to inhibit either tumor growth or subdue inflammation.

The tumor neovascularization field remains challenged by the multiple evasion mechanisms induced in malignancies during antiangiogenic therapies (Bergers G et al. 2008). The discovery of vascular endothelial growth factor (VEGF) (Ferrara N 2004) and VEGF delineation as one of the predominant tumor produced angiogenic factors spawned research into drugs and biologics targeting tumor production, stromal availability, and VEGF receptor signal transduction (Cook K M et al. 2010). Although some patients experience tumor size reductions from available methods, tumor growth eventually resumes. De novo or acquired tumor antiangiogenic therapy resistance can be due to several factors. One evasion mechanism is cancer cell production of untargeted angiogenic factors (Bergers G et al. 2008). Another mechanism is tumor chemo and cytokine endocrine secretion that mobilizes and recruits proangiogenic bone marrow myeloid and immune cells (Ferrara N 2010). Tumor-activated stromal fibroblasts can produce untargeted angiogenic factors. (Crawford Y et al. 2009). Tumors can also shift their growth patterns and invade into tissues by host blood vessel cooption (Leenders W P et al. 2004).

There has been a research interest in targeting tumor neovascularization (Goldman et al. 1998; Triozzi and Borden 2011). A series of papers have indirectly targeted neovessels by vectors engineered for tumor cellular expression of soluble angiogenesis growth factor decoys (Mahasreshti et al. 2001). Another approach focused on neovessel transduction using capsid display of peptides cognate for receptors upregulated on tumor microvessels (Bachtarzi et al. 2011). Another strategy used enhancer/promoters differentially activated in tumor ECs by the tumor microenvironment (Jaggar et al. 1997; Takayama et al. 2007; Dong and Nor 2009). Yet the goal of these past studies was to eradicate tumor neovessels. There has also been research interest in tissue resident stem cells beyond those known to repopulate rapid turnover organs such as the gut and skin (Barker et al. 2010). Past research has also utilized a “typical” cytolytic or apoptotic vector approach of conditionally replicating Ad (CRAd) vectors.

Vascular endothelial cells (ECs) are ideal gene therapy targets as they provide widespread tissue access and are the first contact surfaces following intravenous vector administration. Tumor vasculature can be a conduit for nutrient and oxygen influx and metabolic efflux, however emerging studies demonstrate that the microvasculature and the vascular endothelial cell (EC), can be components for establishment and maintenance of niches for host organ stem cells (Ding L et al. 2012). Tumor stem/initiating cells have been identified in these perivascular niches (Zhu T S et al. 2011). The perivascular niche can be maintained by short range, “angiocrine”. EC growth factor secretion and contact between tumor cells and host microvessels (Butler J M et al. 2010).

The tumor gene therapy field is challenged by several issues; target cell vector transduction, hepatic toxicity due to viral gene expression, and innate and adaptive host vector immune response (Khare R et al. 2011; Duffy M R et al. 2012). Previous studies have failed to investigate vector vascular expression in an extensive panel of host organs, and elucidate global determination of reporter expression distribution throughout the tumor neovasculature.

Gene therapy approaches to the vascular endothelium have exploited several approaches. Vector-host cell transduction was manipulated to produce tumor EC targeting (Reynolds P N et al. 2000; Baker A H et al. 2005). Human recombinant adenovirus serotype 5 (Ad5) is the most frequently used gene transfer system because of its appreciable transgene payload capacity and lack of somatic mutation risk. Adenoviral and adeno-associated vectors have been engineered for capsid display of peptides identified on tumor-activated endothelium, or bispecific antibodies cognate for integrins, selectins, or vessel luminal cell surface receptors (Preuss M A et al. 2008; Bachtarzi H et al. 2011; Nettelbeck D M et al. 2001). Vector pseudotyping using fiberknobs from serotypes other than adenovirus type 5, other animal host species, or fiber replacements, either from other viruses or virus-synthetic chimeric fibers, also achieved EC tropism (Preuss M A et al. 2008; Shinozaki K et al. 2006). However, standard Ad5 vectors predominantly transduce liver but not the vasculature following intravenous administration.

Some studies have focused on the dual goals of liver sequestration inhibition, and hCAR de-targeting concomitant with tumor EC transductional targeting (Bachtarzi H et al. 2011). Conditionally replicative adenoviral vectors were used based on tumor angiogenic factor induced EC proliferation (Peled M et al. 2009; Takayama K et al. 2007). Other efforts centered on transcriptional targeting using DNA enhancer/promoter elements induced in tumor-activated ECs either due to growth factor stimulation or tumor microenvironmental alterations such as hypoxia (Dong Z et al. 2009; Greenberger S et al. 2004; Savontaus M J et al. 2002).

A recent study revealed that human “androgen independent” PGA CSCs can be segregated based on a PSA “low” reporter expression (Qin et al. 2012). Another recent study discovered that PCA stem like cells directly home to the bone marrow (BM) hematopoietic stem cell (HSC) niche. PCA stem cells both physically and biochemically mobilized HSCs out of the niche into the more differentiated hematopoietic progenitor cell (HPC) pool (Shiozawa et al. 2011). This PCA stem cell HSC eviction function was controlled by cell surface CXCR4. Abrogation of PCA stem cell CXCR4-bone marrow niche SDF1 adherence by the CXCR4 blocker, AMD3100 mobilized PCA CSCs into the circulation. Beyond SDF1-CXCR4. other ligand/receptor signaling modules such as Wnt-Frizzled receptor, delta/jagged family Notch ligands/receptors, and sonic hedgehog-patched have all been implicated in PCA metastatic growth (Leong and Gao 2008; Takebe et al. 2011). Ligand decoys have been generated for many of these receptors (Funahashi et al. 2008; Lavergne et al. 2011). Despite the extensive research on PCA CSC isolation and function, multi-directional interactions between the melange of bone niche cellular components regulating CSC maintenance, and metastatic PCA growth have not been investigated in depth.

PCA cells can reach the bone via several routes. In BM, PCA cells can adhere to and traverse sinusoidal ECs (Glinsky 2006). PCA-EC adherence can to be regulated by a combination of integrin αv/β3 and CXCR4 chemokine receptor engagement and signaling. PCA cells express CXCR4 and bone perivascular stromal cells, sinusoidal ECs, osteoblasts, and mesenchymal cells express the CXCR4 ligand, stromal derived factor-1, SDF-1/CXCL12. Bone colonizing PCA cells can also engage a gene expression program termed “osteogenic mimicry” (Chung et al. 2009). PCA cells can upregulate molecules activating both osteoclasts and osteoblasts. Receptor activator of NFkB ligand (RANKL) can engage its RANK receptor on osteoclasts to stimulate bone resorption. PCA parathyroid hormone production can similarly stimulates osteoclasts (Kostenuik et al. 2009). Osteoclastogenesis enhanced bone resorption can release bone matrix bound growth factors such as TGFβ that activate both PCA growth and expansion and osteoblasts to produce bone matrix, leading to increased though abnormal woven bone formation (Ibrahim et al. 2010). Molecules stimulating angiogenesis such as VEGF and basic FGF can be released by osteoclasts from the bone matrix, and from metastatic PCA cells (Morrissey et al. 2008). Collectively, the growth factor/chemokine rich metastatic bone microenvironment can enhance proliferation and upregulate survival pathways that can facilitate PCA chemotheropeutic resistance (Sottnik et al. 2011).

CSC mobilization has been achieved using small molecule receptor inhibitors, but the effect is global rather than niche targeted. Drugs such as AMD3100 are well tolerated but present the specter of indiscriminant HSC mobilization complicating tandem cytotoxic chemotherapy administration. Enhanced bone metastatic tumor growth due to AMD3100-mediated osteoclaslogenesis induction is another example of global off-target effects of systemic administration of stem cell ligand blocking factors (Hirbe et al. 2007).

PCA CSCs can compete with host HSPCs for BM niches (Shiozawa et al. 2011). Recent work used lineage-marked mice to elucidate the specific cell types controlling host HSPC maintenance (Nagasawa et al. 2011; Ding and Morrison 2013; Greenbaum et al. 2013). Lineage tracing has yet to be extensively used to study PCA CSC niche interactions. The cellular niche organization and anatomical relationships of the BM have been recently elucidated. There is a close juxtaposition and/or encirclement of host sinusoidal capillaries by niche components (Nagasawa et al. 2011).

Past research using antibodies and small molecule drugs has focused on ablating or inhibiting the process of tumor neovascularization to starve a tumor of nutrients and oxygen. However, tumors possess multiple redundant pathways evading neovascular ablation. Hence, these strategies have in general failed to achieve survival benefits for cancer patients. Nor does the vascular ablation approach benefit patients with benign but equally morbid or lethal diseases such as autoimmune inflammatory diseases, bone marrow failure, Alzheimer's, amyotrophic lateral sclerosis, or multiple sclerosis.

U.S. Patent Application 2006/0099143, “Antibodies Binding to Human Magic Roundabout (MR). Polypeptides and Uses Thereof for Inhibition Angiogensis,” (Bicknell et al.) describes an antibody that binds to Magic Roundabout and an expression system in a host cell using an adenovirus with a promoter. The expression system encodes a polynucleotide in a suitable host cell to produce the antibody or compound of the invention to inhibit angiogenesis and all diseases associated with angiogenesis using expression of a decoy fragment of the Magic Roundabout (ROBO4) protein. However, this reference is silent about the ROBO4 promoter.

U.S. Patent Application 2010/0222401. “Compositions and Methods for Treating Pathologic Angiogenesis and Vascular Permeability,” (Li et al.) describes methods for producing and screening compounds and compositions capable of modulating the described signaling pathway, inhibiting vascular permeability, and inhibiting pathologic angiogenesis. The signaling pathway described in the application is Robo4 signaling and its ability to inhibit protrusive events involved in cell migration, stabilize endothelial cell-cell junctions, and block pathological angiogenesis. This application discloses that expressing Robo4 using an adenoviral vector and Robo4's expression is endothelial-specific. This application does not teach using an adenoviral vector to target expression to endothelial cells by use of the Robo4 promoter/enhancer fragment.

U.S. Pat. No. 8,394,381, “Antibodies, polypeptides and uses thereof,” (Bicknell et al.) discloses a method of inhibiting angiogenesis in an individual in need thereof by administering an antibody that binds Magic Roundabout (MR) or a fragment thereof that inhibits its endothelial cell migration and/or proliferation.

“A three-kilobase fragment of the human Robo4 promoter directs cell type-specific expression in endothelium,” (Okada et al. Cir. Res. 100: 1712-1722.2007) describes the use of the Robo4 promoter for targeting gene expression from vectors to the endothelial cell.

“Derivation of a myeloid cell-binding adenovirus for gene therapy of inflammation,”Alberti. M. O., et al., PLoS one7:e37812, 2012) discloses an adenovirus comprising MBP, and binding of viruses to primary myeloid cell types. Binding is illustrated for peripheral blood, spleen and lung myeloid cells. However, viral transduction or expression in endothelial cells is not disclosed.

A goal of past vascular-targeted therapies was intratumoral ablation in order to “starve” the tumor of nutrients and oxygen. However, vessel ablative therapies can render the tumor microenvironment hypoxic redox stressed. This altered microenvironment can produce untargeted angiogenic factors either via malignant cell autocrine production, or from host bone marrow (BM) derived cells recruited by endocrine tumoral production. The efficacy of intratumoral vessel “normalization” to increase perfusion and consequently drug and oxygen delivery for enhanced radiosensitivity has been questioned by recent studies.

The targeting efficacies and the therapeutic utility of these approaches were affected by different factors. Some studies were solely performed in cultured BCs (Nettelbeck D M et al. 2001; Yang W Y et al. 2006). Bridging studies tested in vitro transduced ECs in mixed tumor-EC injections (Mavria G et al. 2000). Other approaches used direct injection of vascular-targeted vectors into tumors (Song W et al. 2008). These experimental strategies failed to address the crucial challenge of tumor vessel delivery following systemic administration that is the preclinical translational lynchpin. Prior work engaging systemic vector delivery predominantly used enzymatic luciferase assays of whole tissue that were not linearly quantitative (Takayama K et al. 2007). Studies documenting co-localization frequently presented “coned down” high magnification views of single vessels but failed to evaluate tumor-wide vascular distribution (Bachtarzi H et al. 2011; Varda-Bloom N et al. 2001; Haisma H J et al. 2010).

The concept of enzymatic conversion of an inactive prodrug to an active derivative has been employed in cancer therapeutics. One approach has been to capitalize on host cellular, or cancer cell overexpression of endogenous prodrug converting enzymes such as thymidylate kinase for capecitabine conversion to DNA/RNA nucleotide 5-FU or carboxyl esterase irinotecan conversion to the active topoisomerase inhibitor SN38 (Rivory et al. 1996; Hatfield et al. 2011; Shindoh et al. 2011). Another approach has been to transduce tumor cells directly by local injection of Ad vectors encoding prodrug converting enzymes such as thymidine kinase or cytosine deaminase (CD) (Kaliberov et al. 2006; Fuchita et al. 2009).

Subsequent studies also detected ROBO4 activation in lymphatic endothelium and in hematopoietic stem cells (Smith-Berdan S et al. 2011; Zhang X et al. 2012). ROBO4 function has been controversial, ranging from angiogenesis in zebrafish (Bedell V M et al. 2005), or negative regulation in the mammary gland (Marlow R et al. 2010), to vascular integrity and stabilization (Jones C A et al. 2008). migration inhibition (Park K W et al. 2003) versus stimulation (Sheldon H et al. 2009), and repulsion (Koch A W et al. 2011). At the molecular level, ROBO4 was shown to bind paxillin leading to inhibition of Rae activation and lamellipodial formation via GITI-GAP Arf6 GTPase inactivation (Jones C A et al. 2009). Most of the ROBO4 functions were delineated using Slit proteins as presumptive ligands (Jones C A et al. 2008), however more recent work definitively demonstrated the UNC5B receptor as the ROBO4 binding partner (Koch A W et al. 2011).

Prior research has undergone challenges in discerning the differential upregulation of endogenous ROBO4 expression in tumor activated versus quiescent endothelium because most localization studies have used enzyme reporter genes (Okada Y et al. 2007; Jones C A et al. 2008), through ROBO4 has been implicated as a marker of activated endothelium (Huminiecki L et al. 2002; Seth P et al. 2005).

### SUMMARY

The present inventors have developed methods and compositions that make use of the intact vasculature and the endothelial cells (ECs) contained therein as vehicles for delivery of therapeutic agents in benign and malignant disease. In various embodiments, adenoviral vectors are targeted to vascular endothelial cells. In some configurations, the endothelial cell-targeted adenoviral vectors can provide angiocrine functions and thus can be used to treat malignant and benign diseases. In various embodiments, transgene-carrying adenoviral vectors of the present teachings include the following; 1) adenoviral vectors which selectively enter (transduce) and/or are exclusively expressed in vascular ECs; 2) adenoviral vectors comprising transgenes which encode prodrug converting enzymes which produce active cytotoxic chemotherapy drugs following inactive prodrug administration 3) adenoviral vectors comprising transgenes that convert prodrugs or elaborate conversion product molecules that are secreted by ECs into the tissue microenvironments, 4) adenoviral vectors comprising transgenes that are expressed in ECs and activate EC surface molecules to affect cellular function in an adjacent microenvironment, 5) adenoviral vectors comprising transgenes that inhibit inflammation by sequestration of chemo- or cytokines, or encode molecules stimulating disaggregation of plaque formation in Alzheimer's or other benign diseases.

In various embodiments, the present teachings make use of the fact that the vasculature provides widespread access to diseased tissue. In addition, the vascular endothelial cells are in close approximation of target cells within diseased tissue allowing increased and more specific targeted dosing of therapeutic agents. Furthermore, the vascular endothelium is the first cell type/organ encountered by adenoviral vectors. Thus, systemic intravenous or intraarterial vector injection can target vascular endothelium prior to uptake in nonvascular cells in organs and tissues. In some embodiments, endothelial targeted adenoviral vectors can be engineered for cargo gene expression that can be restricted to disease tissue microenvironments. The microenvironment can include different cell types in addition to the diseased cells. Ancillary cell types can include fibroblasts, inflammatory cells-myeloid cells, macrophages and lymphocytes, and fibroblasts. Collectively the crosstalk between diseased cells and the ancillary cellular collection can change the tissue microenvironment. Such changes can include low oxygen, low pH-high acidity, altered redox potential, and intracellular stress. There can also be DNA regulatory regions-enhancer/promoters that are solely activated by one or more diseased tissue microenvironmental alterations. In various configurations, enhancer-promoters can be engineered into adenoviral vectors to increase transgene expression in diseased compared to normal tissue specificity.

In various embodiments, endothelial-targeted adenoviral vectors of the present teachings can be applied to a variety of diseases, including, without limitation, the following:

Cancer, such as solid organ primary site (site of origin) cancer, in particular brain cancer; solid organ metastatic cancer, including but not limited to bone, lung, liver, and lymph nodes, occult cancer metastatic imaging, hematopoietic cancers, including multiple myeloma, leukemia, lymphoma.

Benign diseases, such as inflammatory diseases including but not limited to rheumatoid arthritis, antherosclerosis, psoriasis, Crohn's disease, ulcerative colitis, Type 1 (juvenile onset) diabetes, inflammatory and degenerative central nervous system diseases including but not limited to: Alzheimer's disease, multiple sclerosis, Parkinson's disease, amyotrophic lateral sclerosis; osteoporosis via endothelial angiocrine osteoclast inhibition alone or combined with concomitant angiocrine osteoblast stimulation, vascular insufficiency/ischemic disease including but not limited to: coronary artery disease, lower limb arteriosclerotic vascular insufficiency (peripheral vascular disease), ischemic stroke, CNS diseases including but not limited to cerebral vasospasm following subarachnoid hemorrhage.

In various embodiments, the present teachings include an adenovirus vector comprising a ROBO4 enhancer/promoter operatively linked to a transgene. In various configurations, the transgene can encode a prodrug converting enzyme. In various configurations, the prodrug converting enzyme can be a cytosine deaminase. In various configurations, the transgene can encode a decoy receptor, such as, without limitation, a decoy receptor that binds at least one angiocrine factor. In various aspects, the transgene can encode a truncated CXCR4 receptor. In some configurations, a ROBO4 enhancer/promoter of the present teachings can comprise a tissue-specific expression control element. In some configurations, a ROBO4 enhancer/promoter of the present teachings can comprise a Tet response element. In some configurations, a ROBO4 enhancer promoter of the present teachings can comprise a hypoxia-response element. In some configurations, a ROBO4 enhancer/promoter of the present teachings can comprise a GABP-binding element.

In various embodiments, the present teachings include an adenovirus vector comprising a chimeric AD5-T4 phage fibritin shaft, a trimerization domain displaying a heptapepide, “myeloid cell-binding peptide” (MBP), and a ROBO4 enhancer/promoter operatively linked to a transgene. Ad.MBP includes MBP displayed at the tip of a “de-knobbed” chimeric fiber (Muro. S., et al. 2004; Alberti, M. O., et al. 2012). This vector was shown to bind specifically to myeloid cells ex vivo but predominantly transduced lung vascular endothelium following systemic administration. (Alberti, M. O., et al. 2013). In various configurations, the transgene can encode, without limitation, a reporter, such as a green fluorescent protein, or a prodrug converting enzyme, such as without limitation, a cytosine deaminase. In various configurations, the transgene can encode a decoy receptor, such as, without limitation, a decoy receptor that binds at least one angiocrine factor. In various configurations, the transgene can encode a truncated CXCR4 receptor.

In various embodiments, an Ad.MBP of the present teachings can provide widespread EC transduction in organs such as lung, heart, kidney, skeletal muscle, pancreas, small bowel, and brain. Accordingly, in some embodiments, the present teachings provide molecular access to hitherto inaccessible organs including brain, small and large bowel mucosa, kidney glomeruli, medulla, and papilla, skeletal muscle, and cardiac subendocardium and myocardium. Thus, in various embodiments, a vector of the present teachings can be used for targeting many prominent and vexing human diseases.

In various configurations, Ad.MBP can retain hepatocyte tropism albeit at a reduced frequency compared with standard Ad5. In various configurations, Ad.MBP can bind specifically to myeloid cells ex vivo. In various configurations, multi-organ Ad.MBP expression is not dependent on circulating monocytes or macrophages. In various configurations, Ad.MBP dose de-escalation can maintain full lung targeting capacity but drastically reduced transgene expression in other organs. In various configurations, swapping the EC-specific ROBO4 promoter for the CMV promoter/enhancer can abrogate hepatocyte expression and can also reduce gene expression in other organs.

In various embodiments, the present teachings include methods of expressing a transgene in an endothelial cell (EC) in vivo. In various configurations, the methods can comprise administering to a mammal an adenovirus comprising a ROBO4 enhancer/promoter operatively linked to a transgene. In various aspects, the transgene can encode a prodrug converting enzyme, such as, without limitation, a cytosine deaminase. In various aspects, the transgene can encode a decoy receptor, such as, without limitation, a decoy receptor that binds at least one angiocrine factor. In various aspects, the transgene can encode a truncated CXCR4 receptor.

In various embodiments, the present teachings include methods of mobilizing at least one of granulocytes, monocytes and lymphocytes from bone marrow. In various configurations, these methods can include administering to a mammal an adenovirus comprising a ROBO4 enhancer/promoter operationally linked to a transgene encoding a truncated CXCR4 receptor.

In various embodiments, the present teachings include methods of mobilizing cancer cells in vivo, in various configurations, these methods can include administering to a mammal an adenoviral comprising a ROBO4 enhancer promoter operationally linked to a transgene encoding a truncated CXCR4 receptor. In various aspects, the cancer cells can be comprised by bone marrow (BM).

In various embodiments, the present teachings include methods of selectively targeting endothelial cells. In various configurations, these methods can comprise administering to a mammal an adenovirus, wherein the adenovirus comprises a chimeric AD5-T4 phage fibritin shall and a trimerization domain displaying a myeloid cell-binding peptide (MBP), and an exogenous promoter operatively linked to a transgene. In various configurations, the exogenous promoter can be or can comprise or consist of a ROBO4 enhancer/promoter. In various configurations, the exogenous promoter can be or can comprise or consist of a Tet-responsive element. In various configurations, the exogenous promoter can be or can comprise or consist of a hypoxia-responsive element. In various configurations, the endothelial cells (ECs) can be selected from the group consisting of brain ECs, kidney ECs and muscle ECs. In various configurations, the transgene can encode a truncated CXCR4 receptor.

In various embodiments, the present teachings include methods of treating a cancer. In various configurations, these methods can comprise administering to a mammal an adenovirus comprising a chimeric AD5-T4 phage fibritin shaft and trimerization domain displaying a myeloid cell-binding pteptide (MBP) and a nucleic acid sequence encoding a truncated CXCR4 receptor, and administering a chemotherapeutic agent. In various configurations, the administration of a chemotherapeutic agent can comprise or consist of administering a therapeutically effective amount of the chemotherapeutic agent.

In various embodiments, the present teachings include use of an adenovirus vector comprising a ROBO4 enhancer/promoter operatively linked to a transgene for the treatment of a disease such as, without limitation, a cancer, such as solid organ primary site (site of origin) cancer, in particular brain cancer; solid organ metastatic cancer including but not limited to bone, lung, liver, and lymph nodes; occult cancer metastatic imaging; hematopoietic cancers, including multiple myeloma, leukemia, or lymphoma. In various embodiments, the present teachings include use of an adenovirus vector comprising a ROBO4 enhancer/promoter operatively linked to a transgene for the treatment of a disease such as, without limitation, a benign disease, such as, without limitation, an inflammatory disease such as rheumatoid arthritis, atherosclerosis, psoriasis. Crohn's disease, ulcerative colitis, Type 1 (juvenile onset) or diabetes. In various embodiments, the present teachings include use of an adenovirus vector comprising a ROBO4 enhancer promoter operatively linked to a transgene for the treatment of a disease such as, without limitation, an inflammatory and degenerative central nervous system disease such as Alzheimer's disease, multiple sclerosis. Parkinson's disease or amyotrophic lateral sclerosis. In various embodiments, the present teachings include use of an adenovirus vector comprising a ROBO4 enhancer/promoter operatively linked to a transgene for the treatment of a disease such as, without limitation, osteoporosis via endothelial angiocrine osteoclast inhibition alone or combined with concomitant angiocrine osteoblast stimulation. In various embodiments, the present teachings include use of an adenovirus vector comprising a ROBO4 enhancer-promoter operatively linked to a transgene for the treatment of a disease such as, without limitation, a vascular insufficiency/ischemic disease such as coronary artery disease, lower limb arteriosclerotic vascular insufficiency (peripheral vascular disease), or ischemic stroke. In various embodiments, the present teachings include use of an adenovirus vector comprising a ROBO4 enhancer/promoter operatively linked to a transgene for the treatment of a disease such as, without limitation, a CNS disease such as cerebral vasospasm following subarachnoid hemorrhage.

In various embodiments, the present teachings include methods of treating a disease or disorder that activates angiogenesis in villous endothelium. In various configurations, these methods can comprise administering to a mammal an adenovirus vector comprising a ROBO4 enhancer/promoter operatively linked to a transgene. In some configurations, a disease or disorder of these embodiments can be selected from the group consisting of inflammatory bowel disease, regional enteritis, inflammatory bowel disease of the colon, infection with toxin producing bacteria, and colon cancer precursor legions of multiple polyposis. In some aspects, a transgene of these embodiments can encode a secreted anti-inflammatory cytokine decoy, in some aspects, a decoy can be selected from the group consisting of soluble TNF-alpha receptor, single chain anti-IL1, single chain anti-IL17 antibody, a bacterial anti-toxin, and an RNAi molecule targeting gene product induced by the activation of the WNT pathway in multiple polyposis. In some configurations, the toxin-reducing bacteria can be selected from the group consisting of Clostridium difficile, Clostridium botulinum, and Shigella.

In some embodiments, the present teachings disclose methods of treating an inflammatory CNS disease in a mammal. In various configurations, these methods can comprise administering to the mammal an Ad.MBP.CMV vector encoding a cytokine decoy. In various configurations, the inflammatory disease can be selected from the group consisting of amyotrophic lateral sclerosis and multiple sclerosis.

In some embodiments, the present teachings disclose methods of treating a degenerative disease in a mammal. In various configurations, these methods can comprise administering to the mammal an Ad.MBP.CMV vector encoding a cytokine decoy. In various aspects, the degenerative disease can be selected from the group consisting of Alzheimer's disease and Parkinson's disease.

In some embodiments, the present teachings disclose methods of stimulating appetite in a mammal. In various configurations, these methods can comprise administering to the mammal an Ad.MBP.CMV vector encoding a secreted molecule that affects the hypothalamic appetite nuclei.

In some embodiments, the present teachings disclose methods of inducing satiety in a mammal. In various configurations, these methods can comprise administering to the mammal an Ad.MBP.CMV vector encoding a secreted molecule that affects the hypothalamic appetite nuclei.

In some embodiments, the present teachings disclose methods of treating myelodysplastic syndrome in a mammal. In various configurations, these methods can comprise administering to the mammal an Ad.RGD.H5/H3.ROBO4 vector, wherein the Ad.RGD.H5/H3.ROBO4 vector produces at least one anti-inflammatory molecule.

In some embodiments, the present teachings disclose methods of treating a genetic disease selected from the group consisting of hemophilia and sickle cell anemia in a mammal. In various configurations, these methods can comprise administering to the mammal an Ad.RGD.H5/H3.ROBO4 vector, wherein the Ad-RGD.H5/H3.ROBO4 vector produces at least one anti-inflammatory molecule.

In some embodiments, the present teachings disclose methods of treating a cancer in a mammal. In various configurations, these methods can comprise administering to the mammal an Ad.RGD.H5/H3.ROBO4 vector, wherein the Ad.RGD.H5/H3.ROBO4 vector produces at least one molecule selected from the group consisting of a molecule that mobilizes metastatic cancer or leukemic stem cells and a molecule producing a chemotherapeutic prodrug converting enzyme.

In various embodiments, the present teachings include an adenovirus vector comprising a ROBO4 enhancer/promoter operatively linked to a transgene for use in the treatment of a disease such as, without limitation, a cancer, such as solid organ primary site (site of origin) cancer, in particular brain cancer, solid organ metastatic cancer: including but not limited to bone, lung, liver, and lymph nodes; occult cancer metastatic imaging; hematopoietic cancers, including multiple myeloma, leukemia, or lymphoma. In various embodiments, the present teachings include an adenovirus vector comprising a ROBO4 enhancer/promoter operatively linked to a transgene for use in the treatment of a disease such as, without limitation, a benign disease, such as, without limitation, an inflammatory disease such as rheumatoid arthritis, atherosclerosis, psoriasis, Crohn's disease, ulcerative colitis, Type 1 (juvenile onset) or diabetes. In various embodiments, the present teachings include an adenovirus vector comprising a ROBO4 enhancer/promoter operatively linked to a transgene for use in the treatment of a disease such as, without limitation, an inflammatory and degenerative central nervous system disease such as Alzheimer's disease, multiple sclerosis, Parkinson's disease or amyotrophic lateral sclerosis. In various embodiments, the present teachings include an adenovirus vector comprising a ROBO4 enhancer/promoter operatively linked to a transgene for use in the treatment of a disease such as, without limitation, osteoporosis via endothelial angiocrine osteoclast inhibition alone or combined with concomitant angiocrine osteoblast stimulation. In various embodiments, the present teachings include an adenovirus vector comprising a ROBO4 enhancer/promoter operatively linked to a transgene for use in the treatment of a disease such as, without limitation, a vascular insufficiency/ischemic disease such as coronary artery disease, lower limb arteriosclerotic vascular insufficiency (peripheral vascular disease), or ischemic stroke. In various embodiments, the present teachings include an adenovirus vector comprising a ROBO4 enhancer/promoter operatively linked to a transgene for use in the treatment of a disease such as, without limitation, a CNS disease such as cerebral vasospasm following subarachnoid hemorrhage.

In various embodiments, the present teachings include use of an adenovirus vector comprising a ROBO4 enhancer/promoter operatively linked to a transgene for the manufacture of a medicament to treat a disease such as, without limitation, a cancer, such as solid organ primary site (site of origin) cancer, in particular brain cancer, a solid organ metastatic cancer including but not limited to bone, lung, liver, and lymph nodes, occult cancer metastatic imaging, a hematopoietic cancer, including multiple myeloma, leukemia, or lymphoma. In various embodiments, the present teachings include use of an adenovirus vector comprising a ROBO4 enhancer/promoter operatively linked to a transgene for the manufacture of a medicament to treat a disease such as, without limitation, a benign disease, such as, without limitation, an inflammatory disease such as rheumatoid arthritis, atherosclerosis, psoriasis, Crohn's disease, ulcerative colitis. Type 1 (juvenile onset) or diabetes. In various embodiments, the present teachings include use of an adenovirus vector comprising a ROBO4 enhancer/promoter operatively linked to a transgene for the manufacture of a medicament to treat a disease such as, without limitation, an inflammatory and degenerative central nervous system disease such as Alzheimer's disease, multiple sclerosis, Parkinson's disease or amyotrophic lateral sclerosis. In various embodiments, the present teachings include use of an adenovirus vector comprising a ROBO4 enhancer promoter operatively linked to a transgene for the manufacture of a medicament to treat a disease such as, without limitation, osteoporosis via endothelial angiocrine osteoclast inhibition alone or combined with concomitant angiocrine osteoblast stimulation. In various embodiments, the present teachings include use of an adenovirus vector comprising a ROBO4 enhancer promoter operatively linked to a transgene for the manufacture of a medicament to treat a disease such as, without limitation, a vascular insufficiency/ischemic disease such as coronary artery disease, lower limb arteriosclerotic vascular insufficiency (peripheral vascular disease), or ischemic stroke. In various embodiments, the present teachings include use of an adenovirus vector comprising a ROBO4 enhancer/promoter operatively linked to a transgene for the manufacture of a medicament to treat a disease such as, without limitation, a CNS disease such as cerebral vasospasm following subarachnoid hemorrhage.

### BRIEF DESCRIPTION OF THE DRAWINGS

FIGS. 1A-B illustrate AdROBO4-EGFP expression showing upregulation of endogenous ROBO4 in orthotopic and xenograft tumors. FIG. 1A illustrates immunoblots of liver (Li), kidney orthotopic (KO) und subcutaneous (SC) xenograft tumors derived from 786-O renal cell carcinoma cells probed with a polyclonal ROBO4 antibody. FIG. 1B illustrates densitometry of ROBO4 protein expression normalized to the endothelial cell (EC) marker VE-Cadherin reveals induction in tumors from both locales.

FIGS. 2A-C illustrates vascular restricted ROBO4-directed reporter expression in kidney orthograft and subcutaneous heterograft tumors. Injection of 1.5×10¹¹ Ad5ROBO4-EGFP (ROBO4) or Ad5CMV-EGFP (CMV) viral particles (vp) in hCAR transgenic: Rag2KO/KO mice produces extensive and intense reporter gene expression localized to microvessel endothelial cells in FIG. 2A subcapsular kidney orthotopic (KO), and in FIG. 2B subcutaneous (SC) xenografts. FIG. 2C illustrates immunoblots and densitometry normalized to either b-tubulin or VE-Cadherin reveal elevated EGFP reporter protein expression in both types of tumor. “K”, host kidney, arrow, glomerular tufts, arrowheads and “T” mark tumor boundaries in left panels whereas arrowheads indicate endothelial tip cells in right upper ROBO4 panels in FIG. 2A and FIG. 2B. Magnifications: 40× and 200×; Red, endomucin/CD31 cocktail; Green, EGFP immunofluorescence. In FIG. 2 and subsequent drawings based on multi-color originals, gray-scale versions of each color channel (red, green and blue) are shown, as well as a composite gray scale that combines all 3 (RGB) color channels. In each case, the top left panel is the red channel, the top right panel is the blue channel, the bottom left panel is the green channel, and the bottom right channel is the composite.

FIGS. 3A-D illustrates that Ad5ROBO4 can transcriptionally target metastatic endothelium. ROBO4-directed expression also differentially detected in circumferential microvessels immediately adjacent to ovarian follicles, asterisks in FIG. 3A and FIG. 3B, but not in stromal microvessels. Ad5ROBO4-directed expression is also evident in most microvessels within a peritoneal 786-O renal cancer metastasis compared to nearly undetectable expression in adjacent host fallopian tube microvessels, asterisks. (FIG. 3D). Magnification of FIG. 3A and FIG. 3B 40×, FIG. 3C 200×, FIG. 3D 100×, Red, endomucin/CD31 cocktail; Green, EGFP immunofluorescence in FIGS. 3A, 3C, and 3D. FIG. 3B EGFP immunohistochemistry and hematoxylin counterstain.

FIG. 4 illustrates Ad5 vector expression in a host organ panel in tumor bearing hCAR:Rag2KO/KO composite mice. Magnification: 10×; Red, endomucin/CD31 cocktail; Green, EGFP immunofluorescence.

FIGS. 5A-B illustrates that warfarin pretreatment detargets liver sequestration. FIG. 5A illustrates widespread high-level hepatocyte EGFP expression in tumor bearing Rag2KO/KO mice injected with 1×10¹¹ vp Ad5CMV-EGFP. FIG. 5B illustrates warfarin pretreatment, 5 mg/kg, on day −3 and −1 prior to vector injection at day 0 markedly decreases the frequency of hepatocyte EGFP expression. Warfarin pretreated detargets liver sequestration. Red: CD31/endomucin, Green: EGFP, Blue: DAP1.A. 10×, B. 200×.

FIGS. 6A-B illustrate warfarin liver detargeting in Rag2KO/KO mice increases the endothelial specificity of the Ad5ROBO4 compared to the Ad5CMV vector. FIG. 6A illustrates injection of 1×10¹¹ vp of Ad5ROBO4-EGFP into mice sans the hCAR transgene essentially abrogates endothelial expression in all organs except for liver and spleen. FIG. 6B illustrates immunoblotting corroborates trace detectable host organ EGFP protein expression in all host organs except for liver and spleen. W(−): vehicle injected mice, W(+): mice treated with day −3/−1 warfarin prior to vector injection. Magnification 100×; Red, endomucin/CD31 cocktail; Green, EGFP.

FIGS. 7A-C illustrate warfarin liver detargeting enhances tumor neovascular endothelia cell specificity of the Ad5ROBO4 vector. The Ad5ROBO4 vector mediated sporadic but easily detectable tumor endothelial cell EGFP immunofluorescence in both FIG. 7A orthotopic and

FIG. 7B subcutaneous 786-O tumors grown in vehicle-treated Rag2KO/KO mice. FIG. 7C illustrates that EGFP immunoblotting and densitometry reveal warfarin-mediated reporter expression in both tumor types concomitant with decreased liver expression. Arrowheads; tumor-kidney boundary, rectangle: area of low power image detailed in adjacent panel. Magnifications: 40 and 100×; Red, endomucin CD31 cocktail; Green, EGFP.

FIGS. 8A-B illustrate Ad5ROBO4 retargets liver expression to hepatic ECs following IV injection. FIG. 8A illustrates Ad5CMVEGFP, FIG. 8B illustrates Ad5ROBO4-EGFP. Red/Green/Blue as above. Magnification FIG. 8A 100×, FIG. 8B 200×. An embodiment of the EC targeted Ad vector can detarget the liver for transgene expression.

FIGS. 9A-C illustrates Ad5ROBO4 tumor EC expression. Red: CD31/endomucin, Green: EGFP, Blue: DAPI. FIG. 9A illustrates subcutaneous 786-O tumor. FIG. 9B and FIG. 9C illustrate “Krukenberg” intra-ovarian 786-O metastases. Arrowheads: tumors. Asterisks host ovarian follicles. Red/Green/Blue as above. Magnification FIG. 9A and FIG. 9B 100×, FIG. 9C 200'. Tumor EC expression bias produces widespread intratumoral EC expression.

FIG. 10 illustrates Ad5ROBO4-EGFP intra-, and peri-tumoral marrow expression in an IGR-CaP1 tibial metastasis. Green line outlines proximal tibial tumor. Red: CD31/endomucin, Green: EGFP, Blue: DAPI, 100×.

FIG. 11 illustrates EC angiocrine-targeted Ad vector strategy.

FIG. 12 illustrates femur BM from a CXCL12-GFP mouse. Investment of bone sinusoidal vascular ECs by CAR-EGFP cells. The ECs (Red) are ensheathed by CXC12-Abundant Reticular (CAR) cells (Green). Blue: DAPI. 400×.

FIG. 13 illustrates an embodiment of an EC targeted prodrug-converting enzyme Ad vector Ad5ROBO4-bCDD314A.

FIG. 14 illustrates vector and dose specific toxicity.

FIG. 15 illustrates focal bone marrow ablation mediated by Ad5ROBO4-bCD production of 5-FU following 5-FC 500 mg/kg BID IP. Red: CD31/endomucin, Green: EGFP, Blue: DAPI. 100×.

FIG. 16 illustrates lineage reporter transgenic mice.

FIG. 17 illustrates strategy for simultaneous quiescence testing of PCA CSCs and host stem cells.

FIG. 18 illustrates metastatic implantation inhibition by liver targeted the AdCMV-sCXCR4/SDF1 ligand decoy.

FIG. 19 illustrates a diagram of an embodiment of an EC-targeted Ad5 SDF1 ligand decoy.

FIG. 20 illustrates Ad5ROBO-sCXCR4 mediated blood and spleen hematopoietic mobilization in C57mice, B: blood, S: spleen, BM; bone marrow.

FIG. 21 illustrates strategy for NOTCH/WNT pathway activation.

FIG. 22 illustrates polycistronic cDNA for creation of a gutless, “theranostic”, Ad vector embodiment.

FIGS. 23A-C illustrate incorporation of MBP into Ad5 increased viral gene expression to vascular beds of multiple host organs. FIG. 23A illustrates immunofluorescence microscopy analysis of vector EGFP expression in host organs following intravenous injection of 1×10¹¹ viral particles (vp) of Ad.MBP.CMV into adult C57BL/6J mice. FIG. 23B illustrates EGFP fluorescence per μm² of tissue section area (FI, fluorescence intensity) in each organ derived from Ad5.CMV-injected mice (n=4 for all organs) versus that from Ad.MBP.CMV-injected mice (n=10 for liver, spleen, heart, kidney, muscle, small bowel, and brain; n=7 for lung, pancreas, and large bowel). FIG. 23C illustrates the percentage of vascular EC area expressing EGFP in each organ derived from Ad5.CMV-injected mice (n=4 for all organs) versus that from Ad.MBP.CMV-injected mice (n=10 for heart, kidney, muscle, small bowel, and brain; n=7 for lung, pancreas, and large bowel). Bar graph is mean +/− standard deviation asterisk: adjusted p<0.05. Magnification: 100×. Red: endomucin/CD31, Green: EGFP immunofluorescence. Blue: DAPI, Li: liver, S: spleen, Lu: lung, H: heart, K: kidney, M: muscle, P: pancreas, SB: small bowel, LB: large bowel, B: brain.

FIGS. 24A-B illustrate that warfarin pretreatment reduced Ad.MBP.CMV liver tropism but did not alter gene expression in other host organs. FIG. 24A illustrates warfarin, 5 mg/kg, on day −3 and −1 before vector injection diminished hepatocyte expression but did not change transgene expression in spleen. FIG. 24B illustrates EGFP fluorescence per μm² of tissue area in each organ derived from warfarin-treated mice (n=3 for all organs) normalized as percentage of the mean value of vehicle-treated or untreated counterparts (n=10 for liver, spleen, heart, kidney, muscle, small bowel, and brain; n=7 for lung) with standard deviation. Spleen (S), lung (Lu), heart (H), kidney (K), muscle (M), small bowel (SB), or brain (B). Asterisk indicates adjusted p<0.05. Magnification: 100×, Red: CD31/endomucin, Green: EGFP immunofluorescence, Blue: DAPI.

FIGS. 25A-C illustrate that systemic administration of a low dose of Ad.MBP.CMV into adult mice produced differential and non-linear reduction in gene expression in host organs. FIG. 25A illustrates EGFP expression in host liver, spleen, lung, and brain following intravenous injection of 1×10¹¹ or 2×10¹⁰ vp of Ad.MBP.CMV into adult mice. FIG. 25B illustrates EGFP fluorescence per μm² of tissue area in each organ derived from the low-dose group (n=6 for each organ). FIG. 25C illustrates normalization of the tissue EGFP fluorescence intensity values in FIG. 25B to the mean value of the high-dose counterparts. Asterisk indicates p<0.05. Magnification: 100×, Red: endomucin, CD31, Green: EGFP immunofluorescence. Blue: DAPI, Li: liver, S: spleen, Lu: lung, H: heart, K: kidney, M: muscle, P: pancreas, SB: small bowel, B: brain.

FIG. 26 illustrates that depletion of circulating monocytes and hepatic and splenic macrophages lead to an increased Ad.MBP.CMV gene expression in the lung without a significant change in gene expression in other organs. Representative flow cytometry plots (left panel) quantifying the FSC-high/SSC-low/CD11b-positive/CD45-positive monocyte population in circulation.

FIGS. 27A-B illustrate Ad.MBP.ROBO4 detargeted hepatocyte expression but reduced levels of vascular EC expression in other host organs. FIG. 27A illustrates EGFP expression following intravenous injection of 1×10¹¹ vp of Ad.MBP.ROBO4 info adult mice. FIG. 27B illustrates the EGFP-positive vascular area analysis was performed as shown in FIG. 23C. Magnification: 100×, Red: endomucin, CD31, Green: EGFP immunofluorescence, Blue: DAPI, Li: liver, S: spleen, Lu: lung, H: heart, K: kidney, M: muscle, SB: small bowel, LB: large bowel, B: brain.

FIGS. 28A-B illustrate that incorporation of MBP into Ad5 detargeted the virus from liver hepatocytes, modestly increased gene expression in splenic marginal zone, and markedly enhanced gene expression in all regions of the brain. FIG. 28A illustrates EGFP expression in liver and spleen following intravenous injection of 1×10¹¹ vp of Ad5.CMV or Ad.MBP.CMV into adult C57BL/6J mice. FIG. 28B illustrates immunofluorescence microscopy analysis of EGFP expression in different regions of the brain following intravenous injection of 1×10¹¹ vp of Ad.MBP.CMV into adult C57BL/6J mice. Magnification: 100×, Red: endomucin/CD31, Green: EGFP immunofluorescence, Blue: DAPI.

FIG. 29 illustrates that Ad.MBP.CMV selectively targeted vascular ECs but not pericytes in multiple host organs. High-power magnification EFI micrographs of tissue sections co-stained with an endomucin/CD31 cocktail (top panels) and an EGFP antibody localized Ad.MBP.CMV transgene expression to vascular ECs by the in lung, heart, kidney, muscle, small bowel, large bowel, and brain. Magnification: 400×, Red: CD31/endomucin for top-row panels. PDGFRβ for middle-row panels, and NG2 for bottom-row panels. Green: EGFP immunofluorescence. Blue: DAPI.

FIG. 30 illustrates Ad.MBP.CMV targeted cell population(s) distinct from CD45-positive or F4/80-positive cells in most host organs. Magnification: 400×, Red: CD45 for top-row panels and F4/80 for bottom-row panels, Green: EGFP immunofluorescence. Blue: DAPI.

FIG. 31 illustrates depletion of hepatic and splenic macrophages by clodronate liposomes. Micrographs show F4/80 expression in liver and spleen from saline-treated mice (veh) or clodronate liposome-treated mice (clod). Magnification: 100×, Red: F4/80, blue: DAPI.

FIGS. 32A-F illustrates induced expression of Ad.MBP.ROBO4-EGFP and Ad.RGD.ROBO4-EGFP vectors in region of ischemia-reperfusion (I/R) in a suture mouse model. FIG. 32A illustrates Ad.MBP.ROBO4 expression in the left ventricular IR region. FIG. 32B illustrates Ad.MBP.ROBO4 expression in left ventricular septum. FIG. 32C illustrates Ad.MBP.ROBO4 expression in right ventricular free wall. FIG. 32D illustrates Ad.RGD.ROBO4 expression in left ventricular I/R region. FIG. 32E illustrates Ad.RGD.ROBO4 expression in left ventricular septum. FIG. 32F illustrates Ad.RGD.ROBO4 expression in right ventricular free wall. Red: vascular endothelial specific immunofluorescence using a CD31/endomucin antibody cocktail. Green: EGFP immunofluorescence, Blue: DAPI nuclear stain, Magnification: 40×.

FIG. 33 illustrates Ad.MBP.ROBO4-EGFP expression in the vascular endothelium of the adductor (thigh) muscle following hindlimb ischemia secondary to femoral artery ligation. Red, Green, Blue as in FIG. 32. Mag: 40×.

FIGS. 34A-C illustrates adenoviral vector expression localized within angiogenic villi in a small bowel resection (SBR) model. FIG. 34A illustrates mice injected with Ad.MBP.ROBO4-EGFP live days post sham surgery. FIG. 34B illustrates endothelial and possible lymphatic expression of the same vector in angiogenic villi post SBR. FIG. 34C illustrates high power view of villous in FIG. 34B (arrowhead) showing colocalized vector transgene expression in angiogenic sprouting endothelium (arrowheads indicate sprouts).

FIG. 34A and FIG. 34B 100×, FIG. 34C 400×.

FIG. 35 illustrates Ad.MBP.CMV vector expression in the vascular endothelium surrounding the hypothalamus (encircled). Red, Green, Blue as in FIG. 32. Mag: 40×.

FIGS. 36A-C illustrates expression of Ad.RGD.H5/H3 vector within the vascular endothelium of human prostate brain metastases in a mouse. FIG. 36A illustrates a histological section that is adjacent to FIG. 36B. FIG. 36C illustrates a prostate brain metastases in another mouse. Asterisks denote metastases, cross uninvolved brain. Red, Green, Blue as in FIG. 32. Mag: 100×.

FIGS. 37A-B illustrates Ad.RGD.H5/H3.ROBO4 vector expression in bone marrow sinusoidal endothelium. FIG. 37A illustrates cortical bone marrow in bone shaft. FIG. 37B illustrates trabecular bone marrow near bone end and cartilaginous plate. Red, Green, Blue as in FIG. 32. Mag: 100%

FIGS. 38A-B illustrates expression of Ad.RGD.ROBO4-EGFP in a IGR-CaP1 human prostate cancer femoral bone metastases in NOD/SCTD/IL2Rγ immunodeficient mouse. FIG. 38A illustrates an adjacent section to FIG. 38B. Green and yellow (top of picture) asterisks are hematopoietic cells adjacent to metastasis. White and black (bottom of picture) asterisks are de novo, osteoblastic bone. While and black crosses are metastatic cells. Arrowhead delineates osteoblastic “rimming”, a pathological hallmark of osteoblastic metastases. Red, Green, Blue as in FIG. 32. Mag; 100×.

FIGS. 39A-D illustrates angiocrine production of 5-fluorouracil (5-FU) from bone marrow sinusoidal endothelial cells expressing cytosinc deaminase (bCD) from an Ad.ROBO4 vector. FIG. 39 illustrates bone trabecular histology from a mouse injected with Ad.ROBO4-EGFP control virus. FIG. 39B illustrates corresponding vascular marker immunofluorescence. FIG. 39C illustrates bone trabecular histopathology 5-FC treated mice following Ad.ROBO4-bCD and preinjection warfarin to detarget liver hepatocyte vector sequestration. FIG. 39D illustrates vascular immunofluorescence demonstrating dilated but intact vasculature and apoptotic hematopoietic cells. Red and Blue as in FIG. 32. Mag: 100×.

### DETAILED DESCRIPTION

### Abbreviations

Ad adenoviral/adenovirus
Ad5 adenovirus serotype 5
ANOVA analysis of variance
BM bone marrow
CAR Coxsackie and adenovirus receptor
CMV cytomegalovirus
CSC cancer stem cell
DAPI 4′,6-diamidino-2-phenylindole
EC endothelial cell
EGFP enhanced green fluorescent protein
GFP green fluorescent protein
HPC hematopoietic progenitor cell
KO knock-out or kidney orthotopic
PCA prostate cancer
PVDF polyvinylidene difluoride
RCC renal cell cancer
ROBO4 Magic Roundabout
SC subcutaneous
VE-Cadherin vascular endothelial cadherin
VEGF vascular endothelial growth factor
vp viral particles

The present inventors have found that an angiocrine niche can affect angiogenic inhibitor resistance, and can provide a focal microenvironment for selection of aggressive tumor emergence. They thus modified vascular endothelial angiocrine functions for malignant and benign disease treatment using endothelial targeted adenoviral vectors.

The present inventors exploited the intact vasculature and the endothelial cells contained therein as a vehicle for delivery of therapeutic agents in benign and malignant disease. The vasculature can provide access to diseased tissue and the vascular endothelial cells are in close approximation of target cells within diseased tissue which allows for increased and more specific targeted dosing of therapeutic agents. The vascular endothelium is the first cell type/organ encountered by adenoviral vectors. Thus, systemic intravenous or intraarterial vector injection can target vascular endothelium first prior to uptake in nonvascular cells in organs and tissues.

In some configurations, an endothelial targeted adenoviral vector can be modified for cargo gene expression that is restricted to disease tissue microenvironments. The microenvironment can include different cell types in addition to the diseased cells. These cell types can include but are not limited to ancillary cell types including fibroblasts, inflammatory cells, myeloid cells, macrophages and lymphocytes, and fibroblasts. Collectively the crosstalk between diseased cells and the ancillary cellular collection can alter the tissue microenvironment. These alterations can include but are not limited to low oxygen, low pH high acidity, altered redox potential, and intracellular stress. In some embodiments DNA regulatory regions-enhancer/promoters that are solely activated by one or more diseased tissue micro-environmental alterations can be employed. These enhancer promoters can be engineered into adenoviral vectors to increase diseased compared to normal tissue specificity.

Diseases and/or conditions to which endothelial-targeted adenoviral vectors of the present teachings can be applied can include but are not limited to cancers, such as without limitation solid organ primary site (site of origin) cancer, brain cancer, solid organ metastatic cancer including but not limited to bone, lung, liver, and lymph nodes, occult cancer metastatic imaging, hematopoietic cancers including but not limited to multiple myeloma, leukemia, and lymphoma; benign diseases; inflammatory diseases including but not limited to rheumatoid arthritis, atherosclerosis, psoriasis, Crohn's disease, ulcerative colitis, juvenile onset diabetes and Type 1 diabetes, inflammatory and degenerative central nervous system diseases including but not limited to Alzheimer's disease, multiple sclerosis, Parkinson's disease, and amyotrophic lateral sclerosis, osteoporosis via endothelial angiocrine osteoclast inhibition alone or combined with concomitant angiocrine osteoblast stimulation, vascular insufficiency/ischemic disease including but not limited to: coronary artery disease, lower limb arteriosclerotic vascular insufficiency (peripheral vascular disease), and ischemic stroke, and other central nervous system diseases including but not limited to cerebral vasospasm following subarachnoid hemorrhage.

In addition to perfusion for nutrient and oxygen provision, endothelial cells (ECs) can produce and secrete growth factors, chemo- and cytokines into their local microenvironment. This EC function can regulate other stromal cells such as fibroblasts, inflammatory cells, organ parenchymal cells. ECs can regulate adjacent cells by “appositional” signaling that includes direct attachment of adjacent cells to the abluminal EC surface and engagement of membrane tethered growth factors, receptors, and other EC surface molecules that interact with receptors on the adjacent stromal and organ parenchymal cells. Cancer or benign cells (in particular cancer or organ stem cells) can also be regulated by these EC angiocrine functions.

Embodiments of the present teachings include the structure and use of adenoviral vectors carrying transgenes. Configurations can include adenoviral vectors that can selectively enter (transduce) and/or can be exclusively expressed in vascular ECs. In some embodiments, a vector transgene can encode a prodrug-converting enzyme. An expressed enzyme can produce an active cytotoxic chemotherapy drug following inactive prodrug administration. In other embodiments, a transgene can generate, or prodrugs can elaborate conversion product molecules that are secreted by ECs into the tissue microenvironments. In other embodiments, a transgene can be expressed in ECs and activate EC surface molecules which can affect cellular function in an adjacent microenvironment. In some embodiments, for benign diseases, a vector transgene can encode a molecule that can inhibit inflammation by sequestration of chemo- or cytokines. In some embodiments, a vector transgene can encode a molecule that can stimulate disaggregation of plaque formation in Alzheimer's disease.

In some embodiments, adenoviral vectors can be engineered for EC-specific vector entry (transductional targeting) and/or they can be engineered to contain a DNA enhancer/promoter (DNA regulatory element) that can be specifically expressed in ECs. In other embodiments, adenoviral vectors can be engineered with transgenes that can include but are not limited to promoter independent regulatory elements including microRNA seed sequences, 3′ mRNA stability elements, and/or mRNA elements containing mRNA secondary structure that can be translated in microenvironmentally stressed slates such as hypoxia or altered redox.

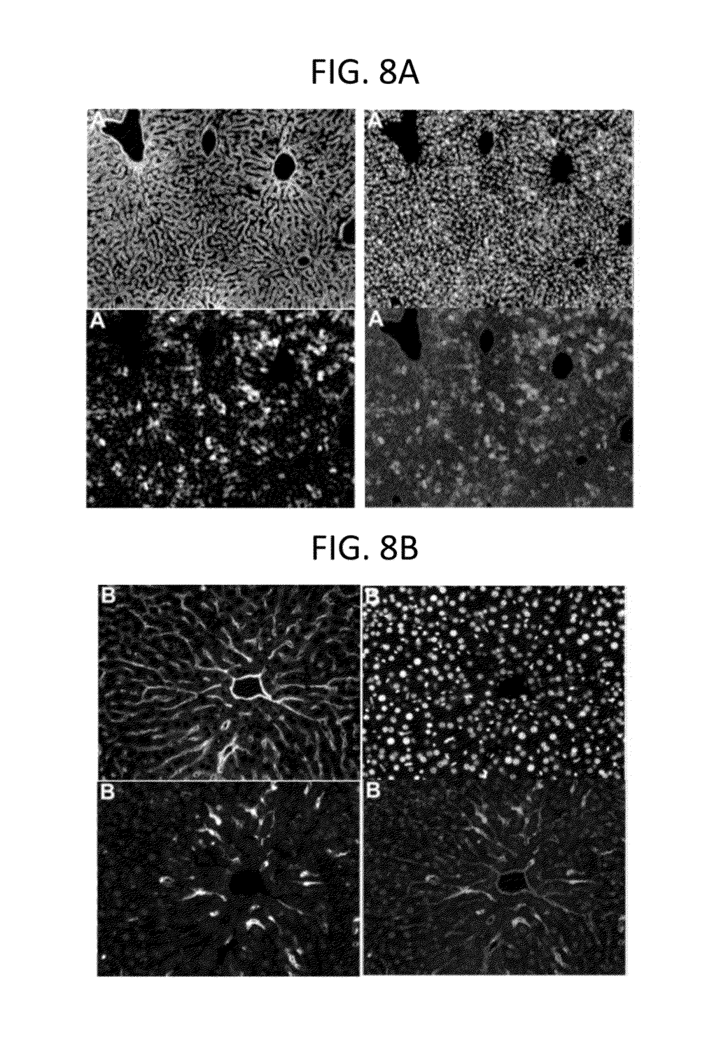

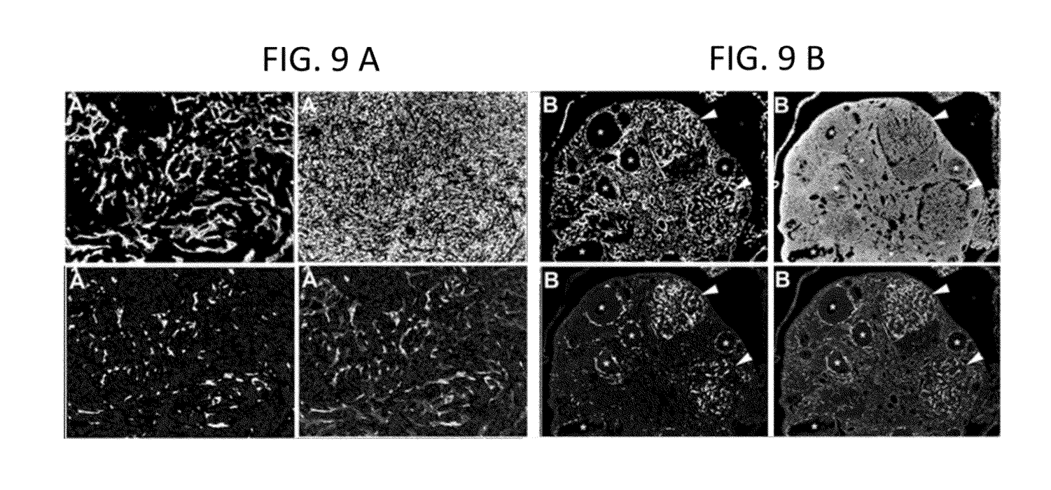

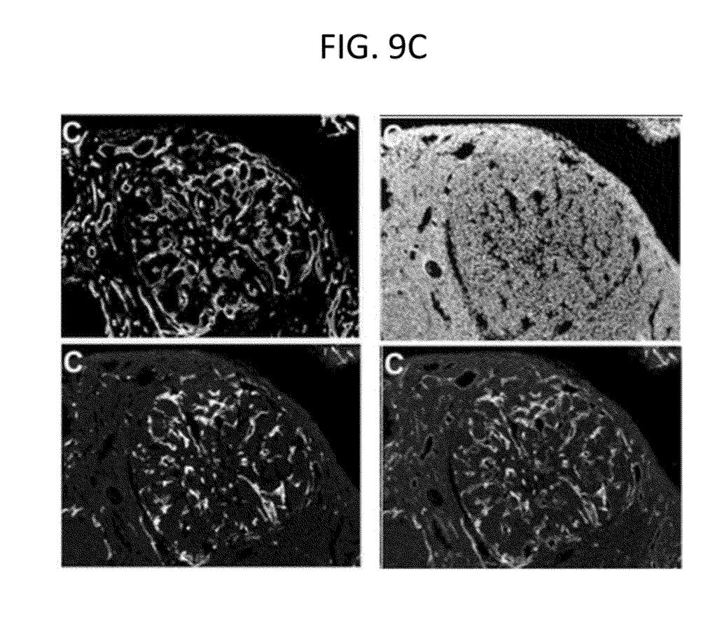

Some embodiments of the present teachings include vector-mediated subversion of endothelial cell (EC) angiocrine functions, which can be used to “cripple” host niche cells that surround ECs and closely appose cancer stem cells (CSCs). In some configurations, the vasculature can be preserved and can redirect ECs to produce secreted molecules in order to dysregulate CSC niche sites throughout bone metastases. In some embodiments, EC-targeted Ad vector configurations can detarget the liver for transgene expression (FIG. 8). In some embodiments, tumor EC expression of the vector configuration bias can produce widespread robust intratumoral EC expression (FIG. 9).

In some configurations, an IGR-CaP1 prostate cancer cell line derived from a Gleason grade 7 radical prostatectomy can grow as gland-forming adenocarcinomas, and form mixed osteoblastic/ostcolytic bone metastases in (immunodeficient) mice (Al Nakouzi et al. 2012). These IGR-CaP1 cells can be androgen independent, and can be enriched for PCA CSC markers (Chauchereau et al. 2011). In some aspects, EC-targeted Ad vector configurations can be expressed within and adjacent to IGR-CaP1 bone metastases (FIG. 10).

In some embodiments, an EC-targeted Ad vector of the present teachings can dysregulate perivascular bone niches which can be essential for CSC maintenance. In some configurations, an Ad vector of the present teachings can control metastatic growth either via enforced CSC differentiation, or by chemo/irradiation therapy synergism due to proliferative transit amplifying cell population expansion. In some embodiments, an Ad vector of the present teachings in combination with bone niche lineage tracing, cell cycle quiescence, and stem cell ligand signaling reporters, can be used to elucidate PCA CSC bone niche dynamics. In some embodiments, angiocrine-targeted Ad vectors can translationally transition to clinical therapeutics.

In some embodiments, the present teachings include use of tumor blood vessels to access the most remote regions of a tumor. In some embodiments, the present teachings include hijacking the perfusion independent “angiocrine” vascular EC functions to produce active drug metabolites or secrete CSC ligand decoys locally and at high levels within bone marrow CSC metastatic niches. In some aspects, this approach can be performed by commandeering EC angiocrine functions using Ad vectors with a predominant metastatic neovascular expression (FIGS. 9, 10). In various configurations, this approach can allow for prodrug end product elaboration specifically within metallic niches for the elimination of systemic toxicities such as stomatitis, diarrhea, or heart failure typical of systemic chemotherapy. In some configurations, an EC-targeted Ad vector of the present teachings can be used to preserve and/or exploit the intralumoral vasculature while avoiding multiple tumor cell autonomous and microenvironmental alterations.

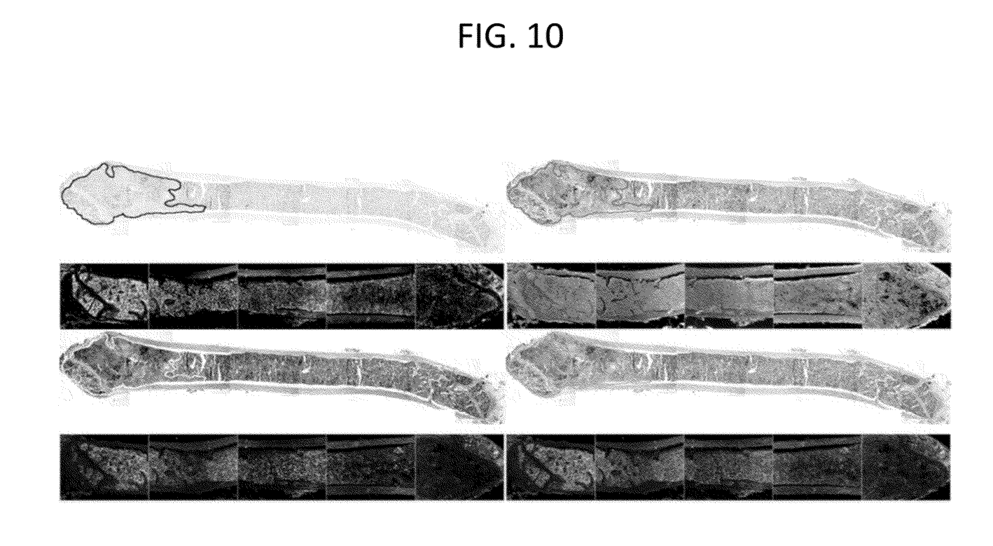

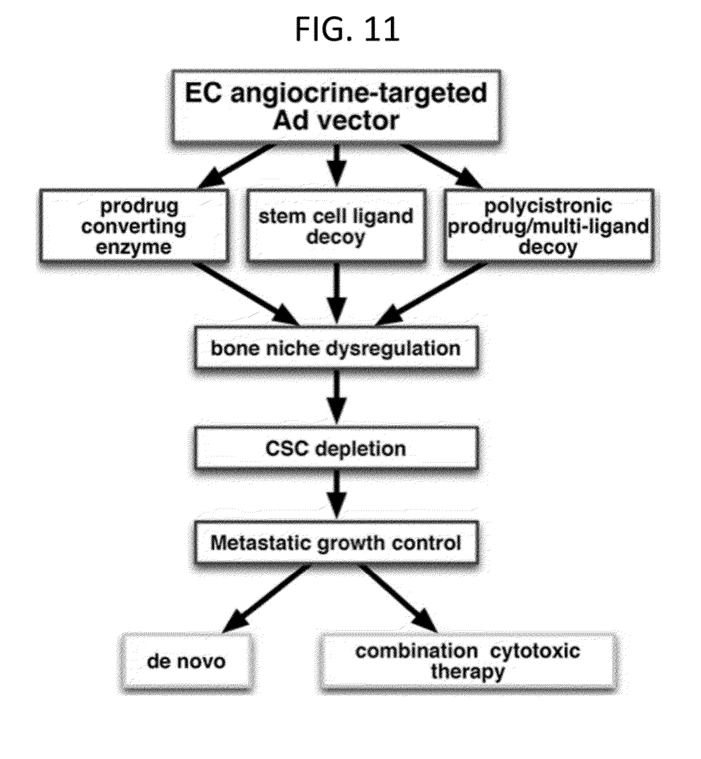

In some embodiments, the present teachings include EC angiocrine secretion modulated by a modified Ad vector (FIG. 11) for targeting metastatic cancer. Ad vector-mediated exploitive engineering of EC angiocrine secretion is a therapeutic strategy for targeting metastatic cancer. (FIG. 11) Metastatic cancer can include, without limitation, prostate cancer, which can metastasize to the bone. In some aspects, multiple niche cellular components within the bone marrow can be targeted. In some aspects, EC targeted Ad vectors of the present teachings can be expressed at high levels in BM sinusoidal EC's both within and adjacent to osteoblastic PCA metastases (FIG. 10). In some aspects, there can be perivascular apposition of niche cellular components combined with bone sinusoidal capillary fenestrations. In some aspects, vectors of the present teachings can be used to dysregulate and disrupt bone PGA CSC niches. In some aspects, bone ECs can be targeted for expression of the 5-fluorouracil (5FU) prodrug converting enzyme, cytosinc deaminase.

In some embodiments of the present teachings, an angiocrine-engineered Ad vector that expresses a stem cell ligand decoy can be used to differentially mobilize PCA CSCs from metastatic bone niches. In some configurations, the CXCR4-SUF1 axis can be disrupted through expression of a decoy such as, without limitation, a truncated NOTCH or WNT ligand decoy.

In some configurations, PCA CSC mobilization effectors can be selected to test combinatorial enhancement of the PCA standard of care chemotherapeutic, docetaxel. In some configurations, Ad-sNOTCH and Ad-sFR/(WNT) ligand decoys in cell culture can be constructed and functionally tested. Combinations of vector embodiments for additive or synergistic PCA CSC mobilization can also be tested. In some configurations, “gutless”polycistronic Ad vectors ligand decoy(s) can be constructed. Such a polycistron can be under switchable control and can obviate constitutive low-level host stem cell mobilization and can provide the potential for a synchronous prodrug-mediated cytotoxic therapy combination. A LUC:GFP fusion construct can be included. In some configurations, a polycistronic vector configurations can be tested in a bone metastatic model. In some aspects, a gutless vector can persist for a prolonged period following a single systemic administration, and can elicit minimal preformed immune responses. In some aspects, a large gutless vector configuration transgene capacity can offer thernanostic potential for combining therapeutic and imaging capabilities into one vector embodiment.

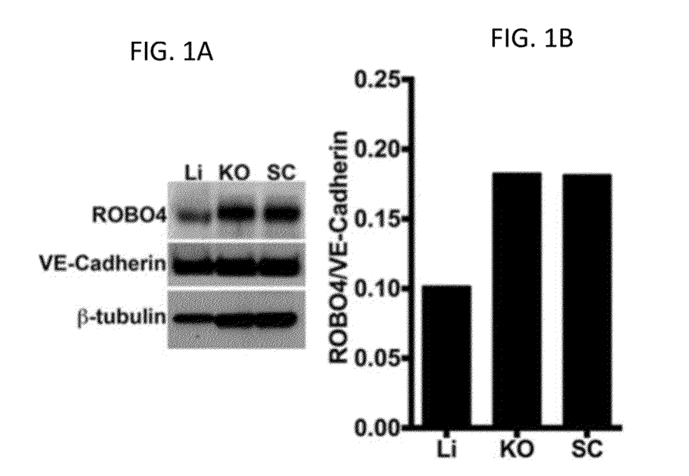

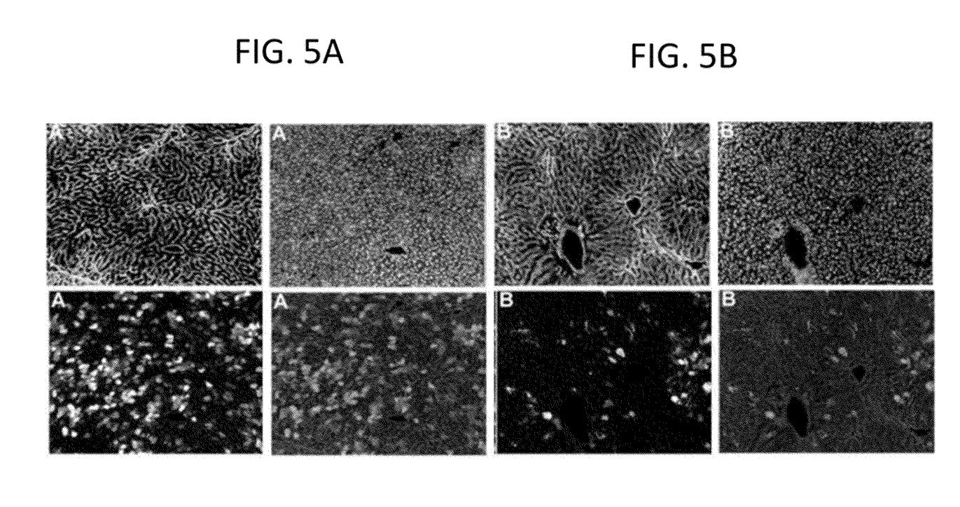

Some configurations include Ad vectors with EC specific expression (FIG. 8, FIG. 9, FIG. 10). In some aspects, these modified vectors can include a 3 kb ROBO4 enhancer/promoter (Okada et al. 2007). The ROBO4 enhancer/promoter fragment can include multiple ETS and hypoxia-inducible factor hypoxia response elements (Okada et al. 2007; Okada et al. 2008). These elements can impart an expression bias for intra- and peritumoral vasculature (FIG. 9, FIG. 10). Most of the AdROBO4 vector can be sequestered in the liver (FIG. 5) (Waddington et al. 2008). Liver sequestration can be predominantly mediated via coagulation factor binding to the adenovirus capsid (Waddington et al. 2008). liver-detargeting efficacy of warfarin pretreatment in mouse models (FIG. 5) can be validated. The AdCMV vector configuration was used to visualize hepatocyte reporter expression. 786-O renal cell carcinoma (RCC) cells were used. There was an induction of AdROBO4-EGFP expression in primary xenograft and metastatic ECs (FIG. 9 and FIG. 10). In contrast, host organ expression of the Ad5ROBO4 vector was restricted to scattered ECs within liver and spleen. Western blotting and densitometry normalized to the EC-specific VE-Cadherin revealed that Ad5ROBO4 reporter expression was greater in tumor versus liver (FIG. 1). Liver detargeted EC targeted Ad vector configurations can be used for therapeutic purposes (Short et al. 2010).

In some configurations, PCA cell lines such as PC3, and LNCaP as PCA models can be tested. Data reveal exquisite EC tropism of MBP vector embodiments in the vasculature of several host organs. The CMV promoter used in these experiments mediated this host organ EC expression. The MBP vector has EC specificity conferred by vector entry (transduction).

In some embodiments, Ad vectors can be tailored for enhanced or restricted tumor EC specificity by choosing from a menu of promoters solely or preferentially activated by the tumor microenvironment. These include but are not limited to promoters activated by hypoxia (Heidenreich et al. 2000; Greenberger et al. 2004; Marignol et al. 2009), DNA damage (Economopoulou et al. 2009; Westerink et al. 2010), or endoplasmic reticulum stress (Zeng et al. 2009; He et al. 2010), all of which are induced in ECs within tumors.

In some embodiments. AdROBO4 can be shown to direct expression in three host organs (liver, spleen, and bone marrow). In various embodiments, PCA bone metastases elicited a peritumoral recruitment of Ad vector expressing ECs ROBO4 can achieve sufficient bone metastatic specificity.

ECs are niche components. ECs are the source of secreted growth factors, chemokine ligands, and membrane tethered molecules that maintain CSC persistence. This short range signaling has been designated as “angiocrine” functions. The present inventors have created EC targeted Ad vector configurations that have angiocrine activity, including in bone. Angiocrine-targeted Ad vectors can be used to achieve metastatic growth control via CSC depletion either alone or in combination with cytotoxic therapies. In some configurations, Multifunctional “theranostic” Ad vectors can be created with translational applicability. In various embodiments, promoter and promoter fragments can be utilized in the Ad vector embodiments for target functions. Tumors, including bone metastases, can be hypoxic. Promoter fragments from VEGF or endothelin; both of which contain hypoxia response elements cognate can thus be used for hypoxia-inducible factor −1 and −2 (Heidenreich et al. 2000; Greenberger et al. 2004). In some embodiments, tumor vasculature can be under DNA damage stress (Economopoulou et al. 2009). In some embodiments, the RAD51C promoter upstream of major DNA repair enzyme can be used to induce for DNA repair (Westerink et al. 2010).

In some aspects, tumor vessels can also be under endoplasmic reticulum stress (unfolded protein response; UPR) (Zeng et al. 2009). In some aspects, the cognate promoter for the XBP1 transcription factor whose alternative splicing is only induced during UPR induction (He et al. 2010) can be used. In some aspects, tumor vascular specificity can be increased with the Ad5ROBO4 vector. In some aspects, micro-RNA seed elements can be placed in the UTR of the CD or any other cargo gene cDNA (Wang and Olson 2009).

In some configurations of the present teachings, there is a family of miRNAs that are dysregulaled in tumor neovasculature (Dews et al. 2006). These seed sites can promote cargo gene mRNA degradation in quiescent host vessels and message stabilization in bone metastasis neovessels. There can also be other RNA structural elements and tumor microenvironment-specific internal ribosome entry sites that can be used, or inclusion of cDNAs encoding peptide elements targeting cargo proteins for degradation in nonmoxic vessels or in host ECs not stressed by increased reactive oxygen species (Oikawa et al. 2012). These fusion proteins can be stabilized in bone metastatic ECs.

An EC-targeted vector configuration, MBP-Ad5 (myeloid binding peptide) (Alberti et al. 2012), can be utilized to provide increased tumor specificity to separate therapeutic efficacy and host (BM) toxicity. Engineering MBP display on the deknobbed Ad5 fiber shaft produces EC transduction (Alberti et al. 2012). EM and in vivo mouse experiments suggests that myeloid cells presented the MBP vector to ECs. Our results show the EC tropism of this vector configuration in the vasculature of several host organs. This host organ EC expression can be mediated by the CMV promoter. In some configurations, Ad vectors can be tailored for enhanced or restricted tumor EC specificity by choosing from a menu of promoters solely or preferentially activated by the tumor microenvironment. These can include promoters activated by hypoxia (Heidenreich et al. 2000; Greenberger et al. 2004; Marignol et al. 2009), DNA damage (Economopoulou et al. 2009; Westerink et al. 2010), or endoplasmic reticulum stress (Zeng et al 2009; He et al. 2010), all of which are induced in ECs within tumors. In some aspects, CSC mobilization potential and cytotoxic chemotherapeutic enhancement of our AdROBO-sCXCR4 SOFt ligand decoy can be utilized.

To demonstrate expression patterns of an Ad vector of the present teachings, an Ad vector containing 3 kb of the Magic Roundabout (ROBO4) promoter transcriptionally regulating an enhanced green fluorescent protein (EGFP) reporter was injected into immunodeficient mice hearing 786-O renal cell carcinoma xenografts and orthotopic tumors.

In some embodiments, the Ad5ROBO4 vector, in conjunction with liver detargeting, can provide genetic access for in-vivo EC genetic engineering in malignancies. Ad5ROBO4-EGFP tumor EC expression was revealed in hCAR transgenic Rag2knockout mice. In contrast, Ad5CMV-EGFP was not expressed in tumor ECs.

As the hCAR transgene also facilitated Ad5ROBO4 and control Ad5CMV vector EC expression in multiple host organs, follow-on experiments engaged warfarin-mediated liver vector detargeting in hCAR negative mice. Ad5ROBO4-mediated EC expression was abrogated in most host organs, while intra-tumoral neovascular expression was spared.

In some embodiments, targeting tumor EC signaling pathways that encompass both angiocrine and perfusion functions can target the multi faceted resistance mechanisms of malignancies. Adenovirus (Ad) is a potential delivery vehicle for tumor EG targeting (Lindemann D et al. 2009; Dong Z et al. 2009). Systemic injection of EC-targeted Ads can circumvent the challenge of tumor permeation vexing local vector injection, and can address the challenge of diffuse, multiorgan, metastatic disease.

In some embodiments, endothelial targeting can be implemented using a configuration of a first generation adenovirus serotype 5 (Ad5) vector. A transcriptional targeting strategy was engaged including creating a vector configuration whose reporter gene was regulated by the endothelial predominant Magic Roundabout (ROBO4) enhancer/promoter. In hypervascular 786-O renal carcinoma xenografts, orthotopic tumors, and spontaneous metastasis. Ad5ROBO4 directed enhanced green fluorescent protein (EGFP) expression to the neovasculature, whereas a vector whose reporter was controlled by the human cytomegalovirus (CMV) enhancer/promoter failed to produce tumor neovascular reporter expression sufficient for detection. Ad5ROBO4 is a vector with the capacity for genetic manipulation of tumor ECs to effect destruction or normalization of the malignant microenvironment.

ECs are one of the primary cells exposed to intravenously injected particles. Tumor microvessels are conduits that can facilitate intra-tumoral vector distribution particularly in hypervascular tumors such as renal cancer metastases. Experiments were performed on vector endothelial transcriptional targeting. A previously characterized 3 kb enhancer/promoter of human ROBO4 (Okada Y et al. 2007) was used to produce vascular endothelial localized gene expression. In some embodiments, an Ad5ROBO4 vector can be used to target the endothelium within primary and metastatic renal cancers, for example in in immunodeficient mice. In various embodiments, vectors and liver detargeted/tumor EC retargeted vectors can contribute to tumor EC-tailored gene therapeutics.

In some aspects, vector reporter gene expression can be quantified using quantitative immunoblotting with a combination of wide field low power and intermediate level microscopic magnification. The latter strategy can demonstrate evidence for vascular EC vector co-localization within primary and metastatic cancers. Wide field imaging can be used to detect heterogeneous vector rumor vessel targeting, these results indicate that combinations of vector configurations tuned to discrete microenvironments can be beneficial for efficacious tumor control.

In some embodiments, tumor microenvironment can selectively activate ECs for ROBO4 expression, as demonstrated by endothelial transcriptional targeting using an Ad5 vector configuration with the ROBO4 enhancer-promoter. An immunoblot analysis can provide evidence for endogenous ROBO4 induction in vascularized tumors compared to normal organs. Immunofluorescence data indicate that the tumor microenvironment selectively activates ECs for ROBO4 expression.

In some aspects, the 3 kb ROBO4 enhancer/promoter fragment used in these studies was analyzed for elements crucial for endothelial specific expression. In some configurations, ETS family and Sp1 transcription factors can mediate endogenous gene induction for ROBO4 enhancer/promoter fragment activity.

In some embodiments, the Ad5ROBO4 capsid can be genetically manipulated to achieve liver detargeting. Ad5ROBO4 vector-mediated tumor EC expression can be demonstrated following factor X-mediated liver detargeting. Our data demonstrate methods of exploitation of CAR independent vector transduction pathways in tumor ECs.

Vascular endothelium has been a sought after gene therapy target because of its immediacy to blood-borne therapeutics and its pathophysiological role in a wide range of benign and malignant diseases. (Dong, Z., et al. 2009; Muro, S., et al. 2004; Lindemann, D., et al. 2009; Aird. W. C., et al. 2007) Despite their accessibility, vascular ECs are poor transduction targets for unmodified Ad5 vectors. (Baker, A. H., et al. 2005) In addition, systemically administrated Ad5 is rapidly opsonized by circulating IgM antibodies and complement components, leading to virus clearance by liver Kupffer cells. (Duffy, M. R., et al. 2012) Ad5 also avidly binds to blood coagulation factor X, which bridges the virus to hepatocytes by interacting with cell surface heparan sulfate proteoglycans. (Waddington, S. N., et al. 2008) Liver Kupffer cell clearance and hepatocyte transduction greatly limit circulating Ad5 vector efficacy. Thus, molecular engineering efforts to achieve Ad vector vascular targeting have focused on diminishing or abrogating liver tropism and opsonization while increasing EC transduction. (Duffy, M. R., et al. 2012; Kaliberov, S. A., et al. 2013) Liver detargeting engaged genetic capsid modification. Virus opsonization diminution has been addressed using chemical shielding of Ad5 capsid proteins. (Duffy, M. R., et al. 2012) One approach to EC transductional targeting has been vector pseudotyping. Ad5 vectors pseudotyped with fibers or fiber knobs from different human, or from non-human, serotypes exhibited improved transduction efficiency of cultured human or rodent (rat) ECs (Shinozaki, K., et al. 2006: Preuss, M. A., et al. 2008; White. K. M., et al. 2013). EC transduction has also been achieved through capsid fiber knob display of peptide ligands such as the arginine-glycine-asparate (RGD) motif cognate for the angiogenesis associated integrins αᵥ/β₅ and αᵥ/β₃, (Preuss, M. A., et al. 2008; Nicklin, S. A., et al. 2001). A parallel strategy for EC specificity has been transcriptional targeting using enhancer/promoter elements of endothelial-specific genes such as VEGFR-2, VEGFR-1, preproendothelin-1, and roundabout-4 (Kaliberov, S. A., et al. 2013; Lu, Z. H., et al. 2013; Song, W., et al. 2005; Greenberger, S., et al. 2004; Reynolds, P. N., et al. 2001; Tal, R., et al. 2008). Transcriptional targeting restricts vector transgene expression to specific EC populations that in most instances are angiogenic and in some cases also hypoxic. However, the transcriptional strategy, when applied alone, docs not alter the Kupffer cell sequestration or hepatocyte transduction. Recent efforts have focused on the combination of transductional and transcriptional strategies to achieve enhanced organ or disease specific EC vector transgene expression. (Kaliberov, S. A., et al. 2013; White, K. M., et al. 2013) Despite progress, systemically administered Ad vectors are still ineffective in gene transfer to some clinically important organs. Dose escalation to achieve appreciable vector expression in marginally accessible organs likely will fail due to dose-limiting adverse effects such as liver toxicity, cytokine storm, or organ imperviousness to the vector. (Zaiss, A. K., et al. 2009) Collectively, the limitations of current EC targeting efforts reinforce the need for further vector improvement.

Ad.MBP was previously shown to preserve the myeloid cell-binding specificity of the MBP peptide ex vivo. (Alberti, M. O., et al. 2012) but efficiently and preferentially target gene expression to the lung microvessel ECs in vivo, (Alberti, M. O., et al. 2013) The latter work used single-cell lung suspensions and confirmed that Ad.MBP solely bound to myeloid cells and not to ECs. Co-culture of virus-loaded myeloid cells on an EC monolayer provided indirect evidence supporting a myeloid cell-mediated viral “handoff” mechanism for potentiating the EC transduction. (Alberti, M. O., et al. 2013) Similar carrier cell hand-off or “hitchhiking” target cell transduction was proposed for other viruses in vivo. (Cole, C. et al. 2005; Roth, S.C., et al. 2008) A central tenet of vector hand-off postulates close contact of virus-carrier cells to the target cells enabling viral penton access with target cell integrins for internalization, bypassing the requirement of an initial attachment step in cell transduction. (Roth, J. C. et al. 2008) Indeed, the previous lung work revealed that the Ad.MBP virions rapidly bound to lung following intravenous injection. There the hypothesis was that vector attachment and “hand-off” to lung ECs was mediated by marginated neutrophils. (Alberti, M. O., et al. 2013) Our current data extend these findings and reveal that the MBP peptide possesses a much broader EC-specific tropism in vivo, and many of the permissive recipient organs exhibit a wide range of tissue-specific forms of resident myeloid cells. Our clodronate study provider evidence that circulating mononuclear cells and tissue resident macrophages in liver and spleen are dispensable or redundant for mediating the Ad.MBP EC expression.

The Ad-MBP vector produces multi-organ vascular expression following warfarin-mediated Factor X depletion. Indeed, previous work demonstrated that Factor X-virus hexon binding “shielded” the vector from peripheral natural antibody-mediated destruction in immunocompetent mice. (Xu, Z., et al. 2013)

Multiorgan expression analysis also enabled us to discover the exquisite lung tropism of our Ad.MBP vector. Viral particle dose reduction essentially eliminated gene transfer to most organs while maintaining robust lung expression. This apparent pulmonary vascular avidity indicates that the Ad.MBP vector can be an ideal vehicle for treatment of pulmonary diseases, particularly those initiated by single gene mutations.

While Ad.MBP has many conceivable applications in other organs, its widespread expression in cardiac and brain vasculature is particularly exciting. In the heart, gene therapy has focused on ischemic disease (Tang, T., et al. 2013). While the immediate cause of cardiac ischemia is coronary artery atherosclerosis, myocardial remodeling is the principal mechanism for development of chronic congestive heart failure (van Berlo, J. H., et al. 2013). Restoration of blood flow has been approached using gene therapy as a surgical adjuvant or as primary treatment (Bradshaw, A. C. et al. 2013; Kaminsky, S. M., et al. 2013). Our Ad.MBP vector can solve the dual challenge of coronary perfusion and myocardial remodeling. Coronary perfusion can be increased using Ad.MBP vector armed with constitutively active hypoxia-inducible factors (HIFs) (Tal, R., et al. 2008). The widespread myocardial vascular distribution of Ad.MBP presents the opportunity to capitalize on EG angiocrine functions such that ECs are transformed into local sources of HIF-mediated angiogenic factors to both preserve marginal zone myocardial viability, and potentially augment arteriogenesis. Similarly, Ad.MBP vectors containing polycistronic transgenes encoding the same molecules apparently secreted by MSCs or ESCs could effect restorative rather than pathological myocardial remodeling by inducing expansion and myocardial differentiation of perivascular resident cardiac stem cells (Kamdar, F., et al. 2012; Ou, D. B., et al. 2013).

Brain gene therapy strives to achieve long-term expression in neurological disorders such as Alzheimer's, amyotrophic lateral sclerosis (ALS), or brain cancer (Coune, P. O., et al. 2012; Ramaswamy, S., et al. 2012; Assi, H., et al. 2012). The ability of our Ad.MBP vector to target greater than 62% of blood vessel beds in all regions of the brain offers the potential for treating the multifocal intraparenchymal mechanisms for both diseases. In brain cancer, glioblastoma (OBM) in particular, the tropism of the Ad.MBP vector for brain vascular ECs can target perivascular GBM stem cells by angiocrine-mediated secretion of secreted cytotoxics or molecules blocking signaling pathways that maintain this therapy resistant cell population (Galan-Moya, E. M., et al. 2011; Zhu, T. S., et al 2011).

The Ad.MBP vector enables unprecedented multi-organ vascular access. This vector can be used to harness ECs for production of a variety of therapeutic molecules for a diverse collection of benign and malignant diseases. Its multi-organ tropism may be uniquely beneficial. In cases wherein greater disease specificity is required, the inherent EC vector tropism allows swapping in enhancer/promoters tailored to the altered microenvironment created by each disease in each organ.

### Materials and Methods

Methods and compositions described herein utilize laboratory techniques well known to skilled artisans, and can be found in laboratory manuals such as Sambrook, J., et al. Molecular Cloning: A Laboratory Manual, 3rd cd. Cold Spring Harbor Laboratory Press, Cold Spring Harbor. N Y, 2001; Spector. D. L. et al. Cells; A Laboratory Manual, Cold Spring Harbor Laboratory Press, Cold Spring Harbor, N. Y. 1998; Nagy, A., Manipulating the Mouse Embryo: A Laboratory Manual (Third Edition). Cold Spring Harbor, N. Y. 2003 and Harlow, E., Using Antibodies: A Laboratory Manual, Cold Spring Harbor Laboratory Press, Cold Spring Harbor, N. Y. 1999. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman. J. G., et al., Goodman & Gilman's The Pharmacological Basis of Therapeutics, Ninth Edition, McGraw-Hill, 1996; and Rowe, R. C., et al., Handbook of Pharmaceutical Excipients, fourth Edition, Pharmaceutical Press, 2003. As used in the present description and any appended claims, the singular forms “a”, “an” and “the” are intended to include the plural forms as well, unless the context indicates otherwise.

### Animals

All mice were of C57BL/6J background and seven to fourteen weeks of age. Mice were obtained from Jackson Laboratory (Bar Harbor, Maine) or through breeding in authors' animal facility, experimental procedures involving mice were carried out under protocols #20120029 and #20110035 approved by the Washington University Animal Studies Committee.

### Cells and Adenovirus Vectors

Human embryonic kidney HEK293 cells were purchased from Microbix Biosystems (Ontario, Canada). Celts were cultured in PMEM/F12 (Mediatech, Hemdon, Va.) media containing 10% fetal bovine serum (FBS) (Summit Biotechnology, Fort Collins, Col.), in a humidified atmosphere with 5% CO: at 37° C. Replication incompetent E1- and E3-deleted Ad5 vectors were created using a two-plasmid rescue method. Plasmids encoded expression cassettes containing either the cytomegalovirus major immediate-early enhancer/promoter (CMV), or the human roundabout4 (ROBO4) enhancer/promoter, each cloned upstream of enhanced green fluorescent protein (EGFP) followed by the bovine growth hormone polyadenylation signal. These expression cassettes were cloned into a shuttle plasmid (pShuttle. Qbiogene, Carlsbad, Calif.) to generate the pShuttleCMV-EGFP and pShuttleROBO4-EGFP plasmids, respectively, and inserts were confirmed by using restriction enzyme mapping and partial sequence analysis. The shuttle plasmids were linearized with Pme 1 and integrated into the Ad5 genome by homologous recombination with a pAd5 plasmid, encoding the native Ad5 fiber, or a pAdMBP plasmid. encoding an MBP-fiber-fibritin chimera, in the E. coli strain BJ5183. To rescue Ad.MBP.ROBO4, the recombinant viral genome was linearized with Pac I and then transfected into 293F28 cells using SuperFect Transfection Reagent (Qiagen, Chatsworth, Calif.). 293F28 cells stably express the native Ad5 fiber thus, viruses rescued at this point were mosaic in the sense that the Ad5 virions randomly incorporated a mixture of native Ad5 fibers and MSP-fiberfibritin chimeras. (Belousova, N., et al. 2003) After an additional round of amplification on 293F28 cells, the viruses were amplified in HEK293 cells, which do not express native Ad5 fiber, to obtain virus particles containing only MBP-fiber-fibritin proteins, the Ad.MBP.CMV vector containing a peptide sequence on a T4 fibritin chimeric fiber knob was created as described previously. (Alherti, M. O., et al. 2013; Alberti, M. O., et al. 2012) Recombinant viruses were purified by two rounds of CsCl density ultracentrifugation and dialyzed in storage buffer containing 10 mmol/L HEPES, 1 mmol/L MgCl₂, pH 7.8 with 10% glycerol as previously described, (He. T. C., et al. 1998) The viral particle (vp) concentration was determined by absorbance of dissociated virus at A260 nm using a conversion factor of 1.1×10¹² vp/absorbance unit.

### Warfarin and Clodronate-Liposome Treatment

Mice were subcutaneously injected with warfarin, 5 mg/kg in peanut oil, 72 hours and 24 hours prior to virus injection. (Short, J. J., et al. 2010) Clodronate-liposomes, 10 μL/g body weight, (ClodronateLiposomes.com. Netherlands) or saline buffer were injected into the tail vein 48 and 24 hours prior to vector injection, (van Rooijen, N., et al. 2010) Twenty-four hours later, peripheral blood was collected by cheek pouch bleeding, and then Ad.MBP was injected.

### Virus Injection and Host Organ Harvest

Mice were tail-vein injected with 1×10¹¹ or 2×10¹⁰ particles of virus in 200 μL of saline. Seventy-two hours post virus administration, mice were anesthetized with 2.5% 2, 2, 2-tribromoethanol (Avertin, Sigma-Aldrich, St. Louis, Mo.), perfused via the left ventricle with phosphate-buffered saline (PBS) followed by 10% neutral buffered formalin. Harvested organs were post-fixed in formalin at room temperature for 2 to 4 hours, cryo-preserved in 30% sucrose in PBS at 4° C. overnight. Lung was further inflated and fixed by injecting formalin solution into trachea followed by closing the trachea by ligature and then processed as above. Treated tissues were embedded in NEG50 (Thermo Fisher Scientific, Waltham, Mass.) or Tissue-Tek OCT mounting medium (Sakura Torrance, Calif., USA), and frozen in a liquid nitrogen pre-chilled, 2-methylbutane-containing glass beaker.

### Immunofluorescence Staining

All mouse tissues were cryosectioned at 16 μm. Lung was also cut at 5 μm for determination of transgene microvessel co-localization. Frozen section slides were air-dried for ten minutes, washed three times in PBS, blocked with protein block solution (5% donkey serum and 0.1% Triton X-100 in PBS) for one hour, and incubated at 4° C. overnight in protein block containing primary antibodies including: rat anti-endomucin 1:1,000, rat anti-PDGFRβ 1:200 (#14-5851-81, and #14-1402-81. Bioscience, San Diego, Calif.). Armenian hamster anti-CD31 1:1.000, rabbit anti-NG2 chondroitin sulfate proteoglycan 1:100 (#MAB1398Z and #AB5320, EMD-Millipore, Billerica, Mass.), rat anti-CD45 1:100 (#550539, BD Biosciences, San Jose, Calif.), rat anti-F4/80 1:500 (#MCA497R, AbD Scrotec-BioRad, Raleigh, N.C.), rabbit anti-GFP 1:400, and chicken anti-GFP 1:400 (#A11122 and πA10262. Life Technologies, Carlsbad, Calif.). The two GFP antibodies performed equally well; the chicken anti-GFP antibody was used in the clodronate-liposome experiment, and the rabbit antibody was used throughout the rest of the study. On day 2, the slides were washed three times in PBS. incubated with corresponding 1:400 diluted Alexa Fluor 488-and Alexa Fluor 594-conjugated secondary antibodies (Jackson ImmunoResearch Laboratories, West Grove, Pa.), and counterstained with SlowFade Gold Antifade mounting reagent with 4′,6-diamidino-2-phenylindole (DAPI) (Life Technologies).

Immunofluorescence microscopy-based analysis of viral reporter gene expression

Immunofluorescence images were collected using an Olympus BX61 microscope equipped with an FVII digital camera (Olympus America, Center Valley, Pa.). The Extended Focal Imaging (EFI) function was used in collecting high-magnification micrographs to allow the creation of a single in-focus image from a series of views of the same field at different z-dimensional focal planes at 2 μm intervals. EFI was carried out in a live-processing mode during image acquisition.

Camera acquisition lime for EGFP immunofluorescence was optimized and set a priori for each organ through independent experiments where the collected data were pooled for statistical analyses. The optimized acquisition time for EGFP immunofluorescence display was 200 msec for liver, 400 msec for spleen, 300 msec for lung, 300 msec for heart, 300 msec for kidney, 1 sec for muscle, 500 msec for pancreas, 1 sec for small bowel, 1 sec for large bowel, and 500 msec for brain. Wherever a figure contains micrographs collected using a different level of exposure for EGFP, the setting is indicated in the figure legend. Immunofluorescence micrographs were subjected to measurement of both color intensity and color-positive area using MicroSuite Biological Suite image analysis software Version 5 (Olympus). To determine the EGFP fluorescence intensity, a threshold defining the background green fluorescence color for each pixel was set at 70 while the possible range of intensity values were from 0 representing a complete absence of green color intensity to 255 taken to be of full intensity. A region of interest (ROI) was drawn over the tissue compartment in each image, and positive ID particles in the ROI, defined as containing at least 5 connected pixels with above the background color intensity, were identified. The color intensity values from every pixel of positive ID particles were summed and normalized by the tissue ROI area (per μm2). To evaluate the fraction of tissue vascular area expressing EGFP, the endothelial marker-positive area and EGFP-positive area within the tissue ROI were quantified by summing up the areas of positive ID particles based on the color detection threshold of 70 for both the green and red colors. The percentage ratio of EGFP-positive area to EC-positive area in each organ was calculated for evaluating the vascular EC vector gene expression. Mean and standard deviation of data points in each organ derived from experiment mice were plotted.

### Quantitative Flow Cytometry

Peripheral blood was collected from mice treated with vehicle or clodronale liposomes and 50 μL of each sample was spiked with re-fluorescent beads (Invitrogen, Calif.) as internal standards for absolute counts. Red blood cells were lysed with red blood cell lysis buffer (BioLegend, San Diego, Calif.), and mononuclear cells (MNCs) were isolated. MNCs were then washed with cold PBS and stained with CD11b-fluorescein isothiocyanatc (FITC) and CD45-phycoerythrin (PE) (BD Phanningen, BD Biosciences, San Jose, Calif.) for 1 hr on ice. Then, cells were washed, resuspended in PBS and analyzed by flow cytometry. Forward scatter (FSC) and side scatter (SSC) were used to gate monocytes as high-size (FSC)/low-granulation (SSC) population; moreover, the monocyte population was further characterized as CD11b-positive/CD45-positive. The count of the FSC-high/SSC-low/CD11b-positive;CD45-positive monocyte population was normalized to the count of the fluorescent beads. Results were presented as the % of average of vehicle treated mice.

### Statistical Analysis

All data are reported as mean ± standard deviation. Significance of the means between the mouse groups was determined using unpaired Student's test for each organ with Bonferroni correction for multiple independent comparisons carried out on different organs from the same cohort of mice. Statistical significance was defined as adjusted P<0.05 (GraphPad Prism, San Diego, Calif.).

Adenoviral vector construction: Replication incompetent E1- and E3-deleted Ad5CMV-GFP and Ad5Robo4-GFP vectors were created using a two-plasmid rescue method. Embodiment plasmids encoded expression cassettes including the human cytomegalovirus (CMV) major immediate-early promoter/enhancer or the magic roundabout (ROBO4) enhancer/promoter elements coupled to the enhanced green fluorescent protein gene, followed by the bovine growth hormone polyadenylation signal. These expression cassettes were cloned into a shuttle plasmid (pShuttle, Qbiogene, Carlsbad, Calif.) and continued using restriction enzyme mapping and partial sequence analysis. The shuttle plasmids were linearized with Pme I enzyme and integrated into the Ad5 genome by homologous recombination with pAdEasy-1 plasmid in E. coli strain BJ5183. Recombinant viral genomes were transfected into HEK293 cells using SuperFect Transfection Reagent (QIAGEN, Chatsworth, Calif.), and packaged into virus particles. Ad5CMV-GFP and Ad5ROBO4-GFP were propagated in HEK293 cells, purified twice by CsCl gradient centrifugation and dialyzed against 10 mM HEPES, 1 mM MgCl₂, pH 7.8 with 10% glycerol. The viral particle (vp) concentration was determined by absorbance of dissociated virus at A260 nm using a conversion factor of 1.1×10¹² vp per absorbance unit.

Generation of composite mice: The Animal Studies Committee of Washington University in St. Louis approved all procedures. Rag-2 knockout (KO) mice (13), in a mixed genetic background, were bred in-house. Transgenic hCAR mice on a mixed genetic background, likely C57B16/J and DBA (14), were obtained from Sven Pettersson. ROSA-R26R knock-in mice were obtained in-house. Rag-2KO/KO mice were serially intercrossed with R26R and hCAR transgenic mice to generate the composite mouse line, hCAR wt:R26R/R26R:Rag2KO/KO, termed hCAR:Rag2KO/KO. The R26R conditional LacZ alleles were not used in these experiments. The warfarin liver detargeting experiments were performed using wt/wt:R26R/R26R;Rag2KO/KO littermates.

Creation of orthotopic and subcutaneous heterotopic tumors: The 786-O human kidney cancer cell line was obtained from ATCC and cultured in RPMI with 10% FBS with pen/strep, amphotericin B. Xenograft tumors were established by injection of 5×10⁶ cells in 50 uL of RPMI media using aseptic technique. Kidney orthotopic tumors were established by left kidney subcapsular injection of 4×10⁶ 786-O cells in 40 uL of RPMI media. Carprofen. 5 mg/kg sc×3 days, (Pfizer Animal Health, NY, N. Y.) was used for postop analgesia. Mice were injected with Ad vectors when the xenograft tumors reached a diameter of about 4 mm.

Ad vector injections, host organ, and tumor harvest: Mice harboring established subcutaneous and kidney tumors were tail vein injected with 5.0×10¹⁰, 1.0×10¹¹, or 1.5×10¹¹ viral panicles of Ad5ROBO4-GFP or Ad5CMV-GFP in 200 μl of saline. For warfarin experiments, mice were administrated warfarin (5 mg/kg) dissolved in peanut oil subcutancously on day −3 and day −1 prior to vector injection. Seventy-two hours post vector administration, mice were anesthetized with 2.5% 2, 2,2-tribromoethanol (Avertin, Sigma-Aldrich, St. Louis, Mo.) perfused via the left ventricle with phosphate-buffered saline (PBS, pH 7.4), followed by 4% para formaldehyde/PBS for whole body fixation. Mouse organs and tumors were collected, post-fixed in 4% paraformaldehyde for 2 hours at room temperature, cryopreserved in 30% sucrose for 16 hours at 4° C., and cryo-embedded in NEG50 (Thermo Fisher Scientific, Wallhum, Mass.) over 2-methylbutane/liquid nitrogen.

### Tissue Harvest and Immunofluorescent Localization of Reporter Gene Expression:

Six teen-micrometer frozen sections were air-dried, washed in PBS, blocked with protein block (1% donkey serum in PBS containing 0.1% Triton X-100), and incubated with primary antibodies including: rat anti-endomucin, 1:1,000, (#14-5851-81 eBioscience. San Diego, Calif.). Armenian hamster anti-CD31, 1:1,000, (#MAB1398Z EMD-Millipore, Billerica, Mass.), and rabbit anti-GFP, 1:400, (#A11122 Life Technologies, Carlsbad, Calif.). After PBS washes, the slides were incubated with corresponding Alexa Fluor 488 and Alexa Fluor 594, 1:400, (Jackson ImmunoResearch Laboratories, West Grove, Pa.) conjugated secondary antibodies and counterstained for nuclei with SlowFade Gold Antifade mounting reagent with 4′,6-diamidino-2-phenylindole (DAPI) (Life Technologies). Fluorescence microscope images were collected using an FVII digital camera with Extended Focal Imaging (EFI) function (Olympus America, Center Valley, Pa.). To quantify the tissue section GFP fluorescence, the areas of GFP(+) cells and dual CD31/endomucin(+) blood vessels were measured and normalized by total tissue area per field. Areas of positive fluorescence were quantified using image analysis software (MicroSuite Biological Suite Version 5, Olympus).

Tissue and whole organ reporter protein expression by immunoblotting: Mice were perfused via the left ventricle with cold phosphate-buffered saline (PBS, pH 7.4) containing 1 mM PMSF (Sigma-Aldrich). Organ tissues and tumors were snap frozen in liquid nitrogen and stored in the liquid nitrogen vapor phase. Frozen tissues were pulverized using a liquid nitrogen-chilled Cell Crusher (Thermo-Fisher), and lysed on ice in radioimmunoprecipitation assay buffer (20 mM Tris-HCl (pH 7.6), 0.15 M NaCl. 1% sodium deoxycholate, 1% NP40, 1 mMEDTA, 1 mM EGTA) supplemented with Protease Inhibitor Cocktail, 1:10, Sigina-Aldrieh) for 30 minutes. Protein lysates were separated on polyaerylamidc gels and transferred to polyvinylidene difluoridc (PVDF) membranes. Protein loading in individual lanes was normalized first to β-tubulin and then VE-Cadheria Membranes were blocked in Tris-buffered saline, TBS, pH 7.6, containing 0.5% Tween 20 (TBST) and 5% nonfat dry milk and incubated in 5% BSA in TBST, containing the following antibodies: rabbit polyclonal anti-ROBO4 (Dean Li, University of Utah), chicken monoclonal anti-EGFP, 1:1,000, (#A10262 Life Technologies), goat anti-VE-Cadherin, 1:400, (#A1002 R&D Systems, Minneapolis, Min.), and polyclonal anti-β-tubulin, 1:20,000, (Abeam, Cambridge, Mass.) overnight.

Membranes were washed three times with TBST and incubated in BSA/TBST with the corresponding IgG-horseradish peroxidase conjugate, 1:5,000, (Santa Cruz Biotechnology, Santa Cruz, Calif.) for 1 hour. After three TBST washes, peroxidase activity was revealed by enhanced chemiluminescence using ECL2 or SuperSignal West Femto Western Blotting Substrate (both from Thermo Scientific) and imaged using a Chemidoc XRS imaging system (Bio-Rad Laboratories, Hercules, Calif.). The immunoblotting was quantified by densitometry with Quantity One one-dimensional analysis software (Bio-Rad Laboratories). Statistical analysis: Significance between groups in the differential fluorescent area experiments was determined using one-way ANOVA with Tukey's correction for multiple group comparisons (GraphPad Prism, San Diego, Calif.).

Our molecular and genetic resource tools enable us to obviate hepatotoxicity, innate and adaptive immunity, reticuloendothelial cell (RES) sequestration, and transgene expression persistence. Hepatic sequestration can be overcome by abrogating Ad capsid hexon and penton blood coagulation factor binding (Waddington et al 2008). Warfarin can be used to achieve this in mice in the short term (FIG. 5), but vectors are also available with hexon and penton mutations (Short et al. 2010). Mutant capsid vectors are a translational bridge to clinical trials (Kim et al. 2012). Our strategy of transcriptional (FIG. 8) or transductional EC targeting circumvents hepatocyte vector transgene expression underlying liver toxicity (Raper et al. 2003). Further diminutions of innate and adaptive immunity can be achieved through additional vector engineering. Our strategy of helper-dependent, “gutless” Ad vectors includes vectors lacking the entire Ad genome save for vector long terminal repeats (Muliammad et at. 2010). The nominal viral DNA within these vectors can minimize innate immunity, and the lack of viral protein expression can evade adaptive immunity. Inhibition of RES sequestration and preexistet neutralizing antibodies can be achieved by tailored capsid polyethylene glycol (PEG) shielding (Zeng et al. 2012). Gutless vectors can also achieve prolonged transgene expression (Kim et al 2001). Recent clinical trials showed the feasibility of safe, non-toxic Ad vector systemic (IV) administration (Nathwani et al. 2011; Brenner et al. 2013).

### EXAMPLES

The present teachings including descriptions provided in the Examples that are not intended to limit the scope of any claim or aspect. Unless specifically presented in the past tense, an example can be a prophetic or an actual example. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

This example illustrates the upregulation of endogenous ROBO4 in renal cancer xenografts and orthotopic tumors.

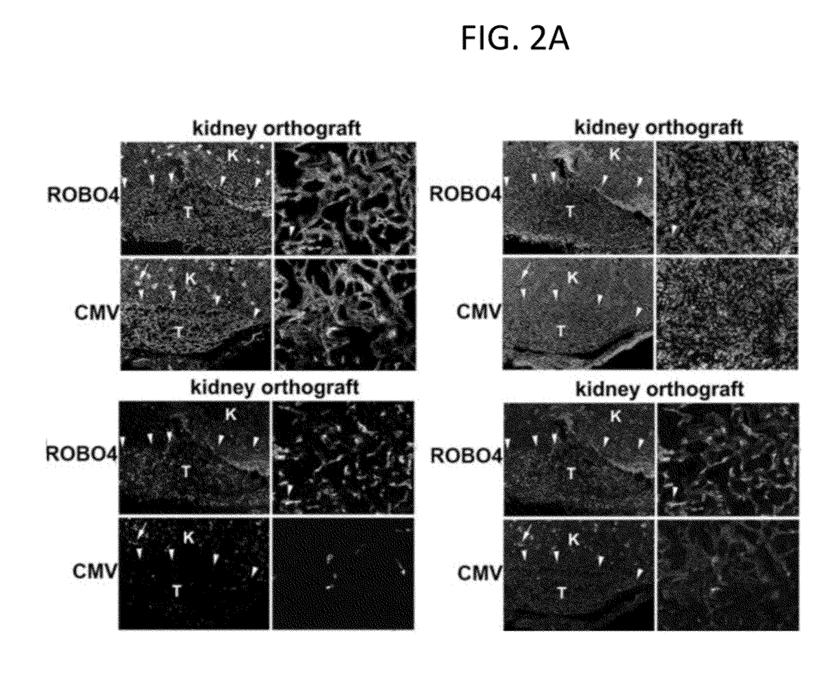

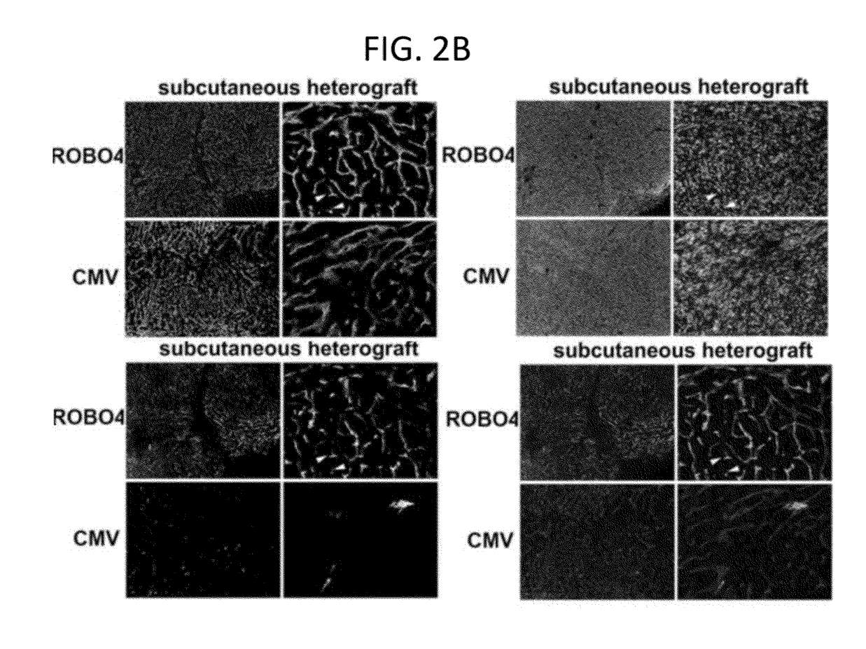

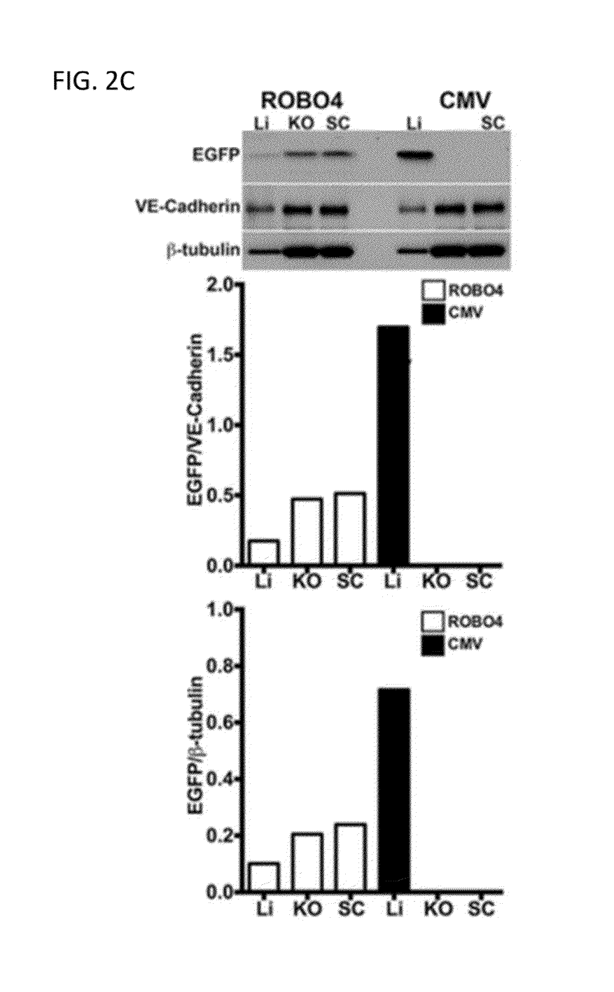

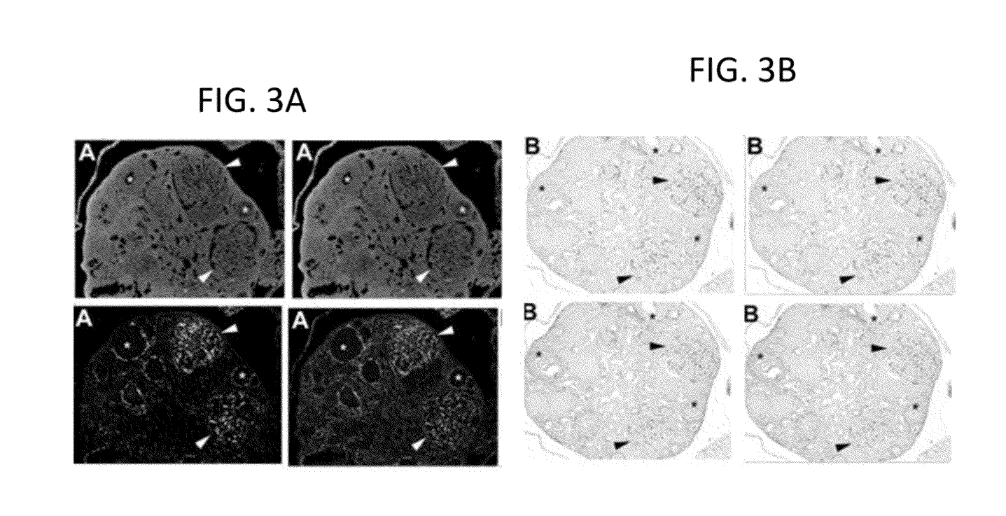

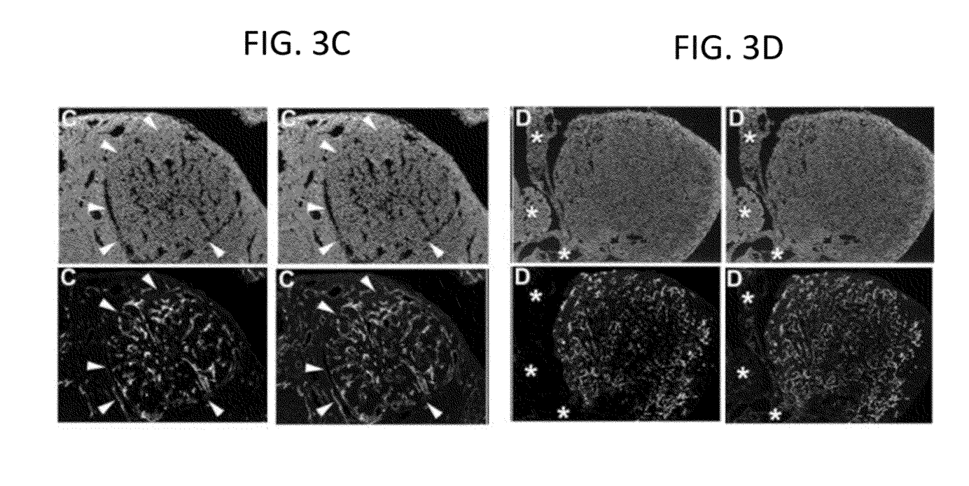

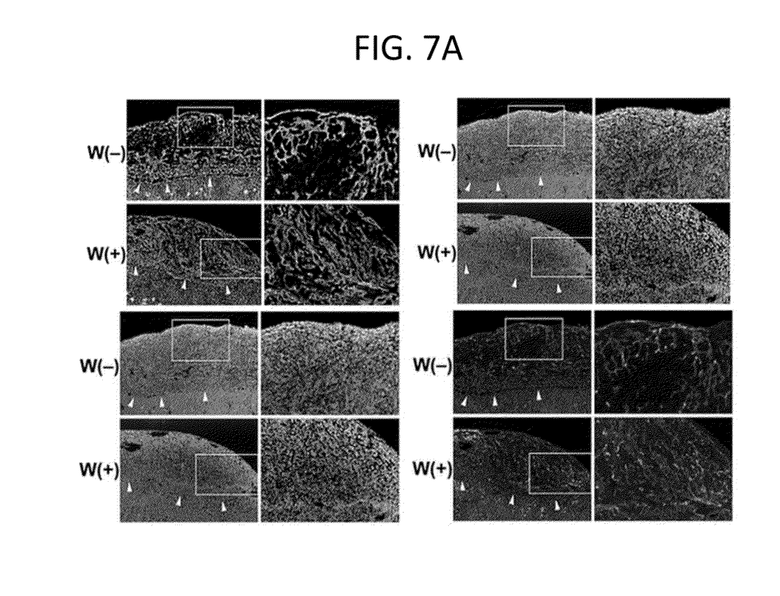

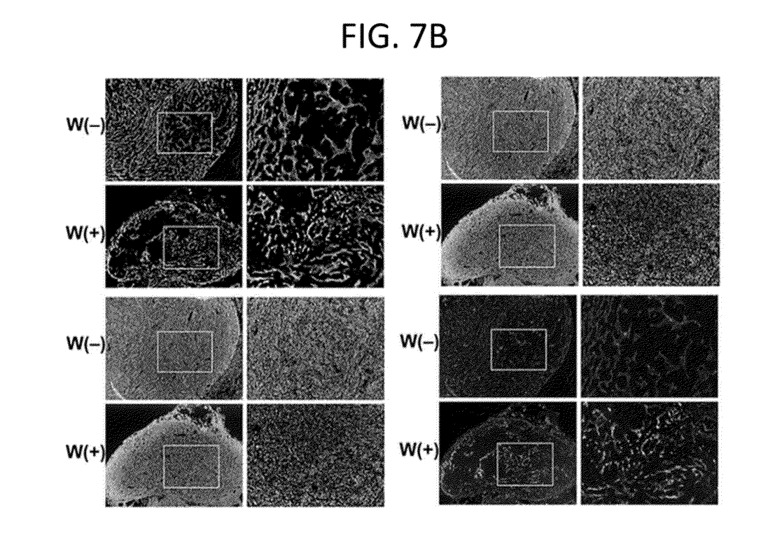

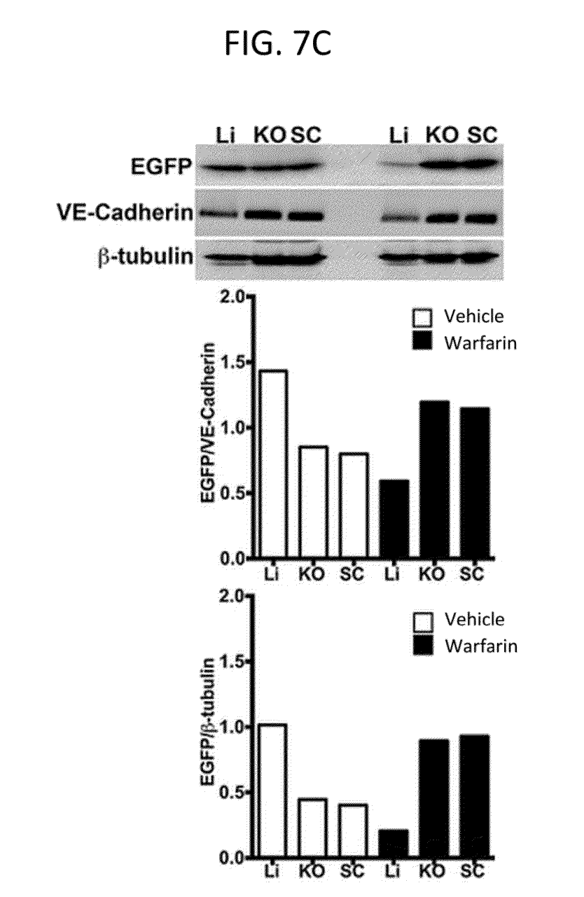

To evaluate Ad vectors for tumor EC targeting, a cancer histotype was selected forming hypervascular tumors both in mouse models and in human disease. Renal cell cancer (RCC) is a paradigm clinical hypervascular tumor whose principal therapy is drugs targeting angiogenesis. The human derived 786-O renal carcinoma cell line was selected because these cells possess the molecular features of, and histologically emulate, clinical renal cell cancer in patients (Kondo K et al. 2003: Gordan J D et al. 2008). In addition, the cells form hypervascular xenograft and orthotopic kidney subcapsular tumors (FIG. 3). It was hypothesized that the vascular ECs in 786-O tumors would be activated (taking into consideration FIG. 7). Warfarin liver detargeting enhanced the multiplicity of tumor endothelial cell reporter gene expression in both tumor locales. (FIG. 7) One candidate gene whose promoter clement could target Ad vectors for EC specific expression in tumor-activated vessels is ROBO4 (Okada Y et al. 2007; Huminiecki L et al 2002; Seth P et al. 2005). Upregulation of the ROBO4 endogenous gene in RCC tumor models was tested. Extracts were immunoblotted from 786-O xenografts, orthotopic tumors, and liver as a control host organ (FIG. 1). The similar levels of vascular endothelial cadherin (VE-Cadherin, Cdh5) expression (FIG. 1) combined with about equivalent vascularity as determined by EC marker immunofluorescence (FIG. 2 and FIG. 3) supports the use of liver as a control host organ for comparison with RCC tumors. Densitometric normalization to VB-Cadherin revealed a 1.8-fold increase in endogenous mouse ROBO4 in both xenografts and orthotopic RCC tumors (FIG. 1). In FIG. 2, FIG. 3 and subsequent drawings based on multi-color originals, gray-scale versions of each color channel (red, green and blue) are shown, as well as a composite gray scale that combines all 3 (RGB) color channels. In each case, the top left panel is the red channel, the top right panel is the blue channel, the bottom left panel is the green channel, and the bottom right channel is the composite.

### Example 2

This example illustrates that an Ad5ROBO4 vector transcriptionally targets tumor endothelial cells.

To transcriptionally target an Ad vector to RCC tumor vasculature the 3 kb enhancer promoter fragment of human ROBO4 previously validated for endothelial expression in single copy and endogenous locus transgenic knock-in mice (Okada Y et al 2007) was used. ECs are known to express trace levels of the Coxsackie and adenovirus receptor (CAR) (Reynolds F N et al 2000; Preuss M A et al. 2008). Immunodeficient composite mice were created containing a human CAR (hCAR) transgene and Rag2 gene deletion (Shinkai Y et al. 1992; Tallone T et al. 2001). Reporter gene localization within tumor ECs was tested (FIG. 3). There was a dichotomy in Ad5ROBO4 versus Ad5CMV vector expression pattern in both kidney orthotopic (KO) and subcutaneous (SC) xenograft tumors of mice intravenously injected with 1.5×10¹¹ viral particles (vp) (FIG. 2). Intense EGFP expression is also detected in endothelial tip cells. In contrast, Ad5CMV-directed expression can be detected in host kidney but neither in orthotopic, nor in subcutaneous tumors. Ad5ROBO4-directed expression was restricted to ECs in both kidney and subcutaneous tumors. Ad5ROBO4 endothelial reporter expression distribution was reduced in mice injected with lower. 1×10¹¹ or 5×10¹⁰ vp, dosages, but EC fluorescence intensity was maintained. There was no detectable co-localized expression within either kidney orthograft or subcutaneous tumors in Ad5CMV-EGFP injected mice despite focal glomerular and interstitial peritubular EC expression in the adjacent kidneys of these mice (FIG. 2). Ad5ROBO4 directed expression was endothelial specific, as neither CD45 cells nor pericytes were positive for EGFP expression (FIG. 2).

### Example 3

This example illustrates that an Ad5ROBO4 vector transcriptionally targets metastatic tumor endothelial cells.

During tissue immunofluorescence analysis intra-ovarian and peritoneal metastases were detected in an Ad5ROBO4 injected mouse (1.5×10¹¹ vp) hearing an orthotopic tumor (FIG. 3). Nearly all of the microvessels within the infraovarian and peritoneal metastases expressed EGFP. There was almost no expression within stromal ECs within the metastasis-bearing ovary except for perifollicular microvessels (FIG. 3). Intra-ovarian “Krukenberg” renal carcinoma metastases in hCAR:Rag2KO/KO mice injected with 1.5×10¹¹ vp. FIGS. 3A-3C, arrowheads, from subcapsular 786-O orthografts demonstrate extensive and intense microvessel EGFP expression. Expression was not detectable in ECs within the fallopian tube abutting the peritoneal metastasis (FIG. 3).

### Example 4

This example illustrates the Ad5ROB4 reporter protein expression in orthotopic and xenograft tumors compared to art index host organ.

To quantitatively test for Ad-mediated tumor reporter expression extracts were immunoblotted from both tumor locales and liver, from mice injected with 5×10¹⁰ vp of either the Ad5ROBO4 or Ad5CMV vectors, and probed for EGFP normalized to either VE-Cadherin or β-tubulin. Neither the orthotopic nor xenograft extracts contained detectable EGFP protein as evidenced by the validated immunofluorescent absence of detectable tumor Ad5CMV regulated expression (FIG. 2). Ad5ROBO4-mediated EGFP expression was 2-2.4-fold elevated when normalized to β-tubulin and 2.6-2.8-fold elevated when normalized to VE-Cadherin (FIG. 2). AdCMV-directed liver expression was 7- to nearly 10-fold elevated when normalized to β-tubulin or VE-Cadherin respectively, compared to Ad5ROBO4-regulated expression (FIG. 2). This result demonstrates the ability of EC transcriptional regulation to detarget Ad hepatic expression.

### Example 5

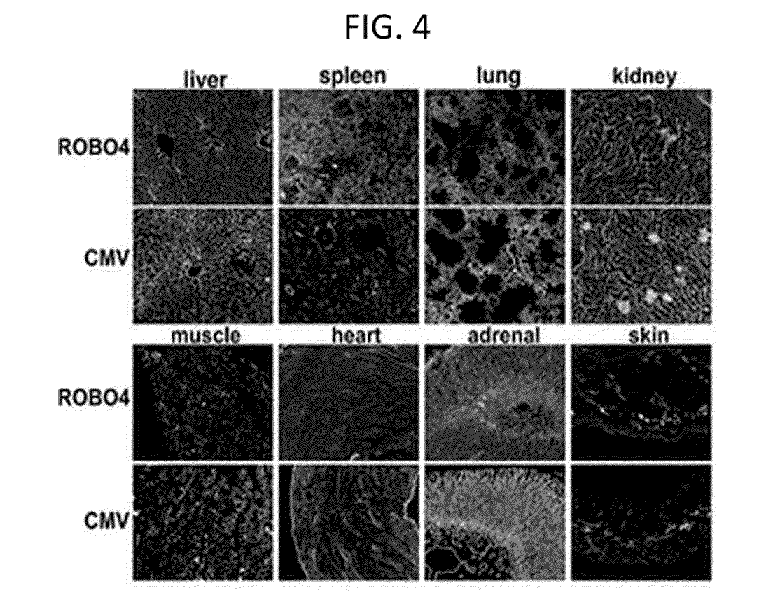

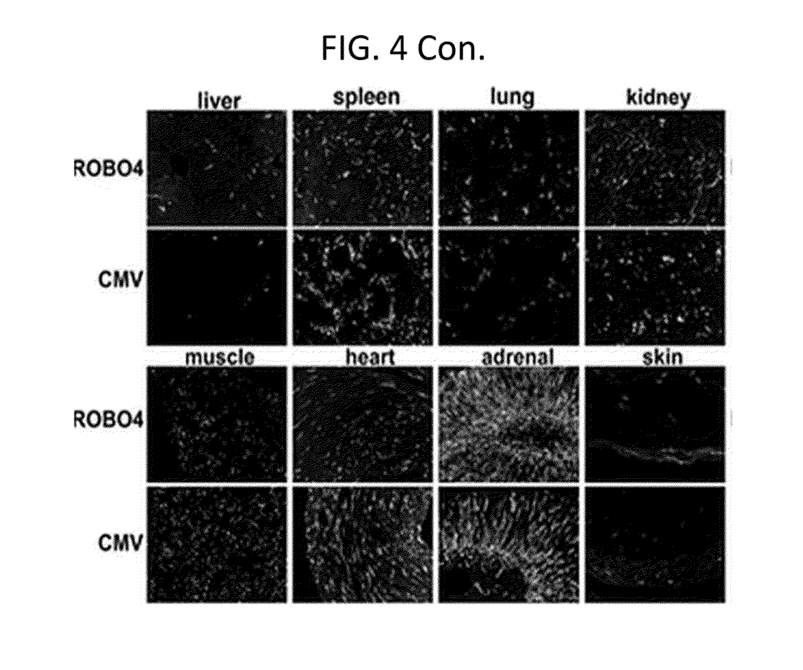

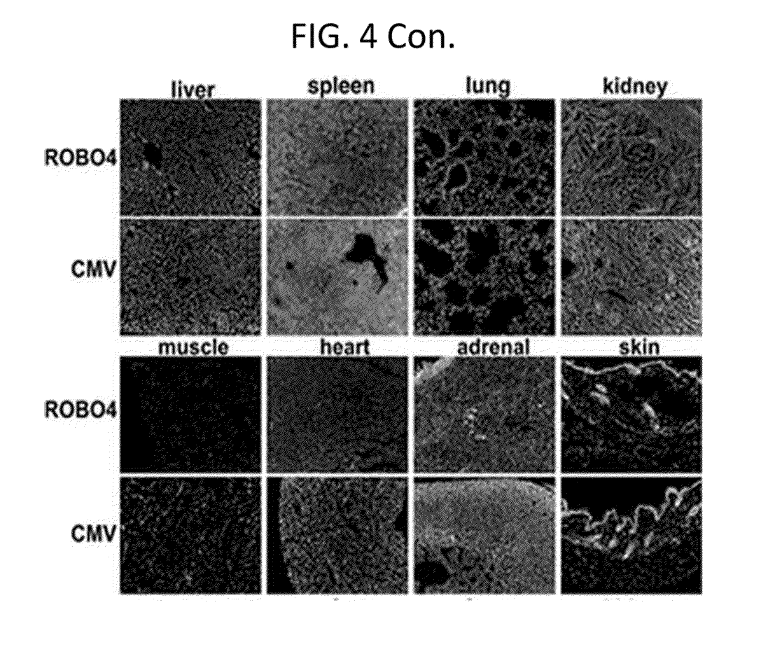

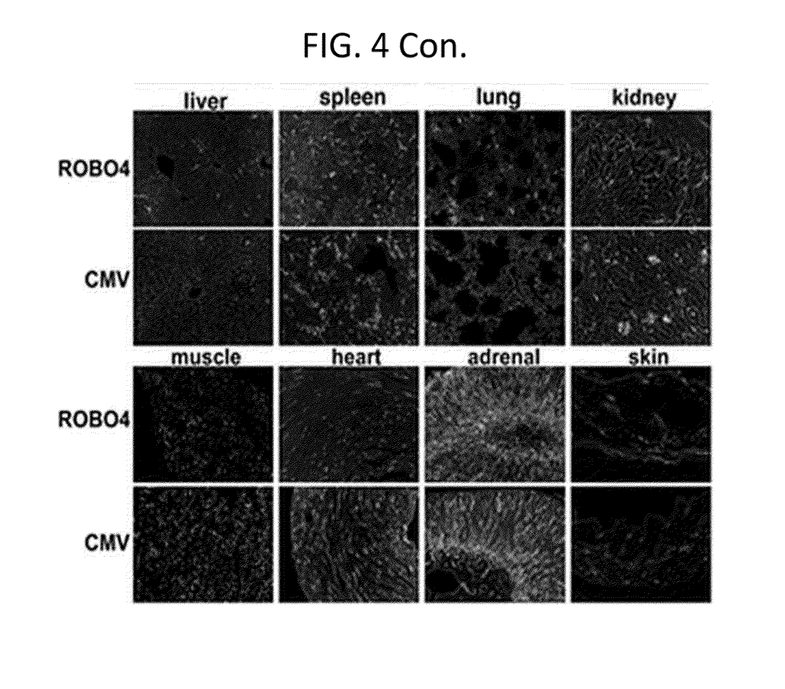

This example illustrates endothelial specific reporter gene expression mediated by both Ad5ROBO4 and Ad5CMV vectors after systemic injection in hCAR transgenic mice. Ad5ROBO4 mediated vector expression was tested using immunofluorescence in a nine organ panel of host organs from the same tumor bearing hCAR:Rag2KO/KO mice as in FIG. 2. Endothelial expression was detected in lung, kidney, muscle, adrenal, heart, skin (FIG. 3), and brain (data not shown) of mice injected with either vector. Both liver and spleen displayed differential cell type localized reporter gene expression mediated by Ad5ROBO4 versus Ad5CMV vectors. In liver, Ad5ROBO4-directed EGFP expression was confined to sinusoidal ECs, whereas Ad5CMV-directed EGFP expression was focally detected in hepatocytes. In spleen. Ad5ROBO4-directed expression was also EC restricted whereas Ad5CMV-directed expression was localized to marginal zone CD16/CD32/F4/80(+) reticuloendothelial cells (FIG. 3). The dose dependency was examined of both the Ad5ROBO4-, and Ad5CMV-EGFP vectors due to EC expression of Ad5CMV-EGFP (in some cases adrenal, heart muscle). Injection of 5×10¹⁰ vp of either vector into hC-AR:Rag2KO/KO mice demonstrated a reduction of heart, kidney, and brain endothelial expression mediated by either vector, and a decrease with retention of adrenal endothelial expression with either vector (FIG. 4). Host organ EGFP reporter expression following intravenous injection of either Ad5ROBO4-EGFP (ROBO4) or Ad5CMV-EGFP (CMV). FIG. 4 illustrates injection of 1.5×10¹¹ viral particles (vp) produced extensive microvessel EGFP expression in both Ad5ROBO4 and Ad5CMV vector treated mice in kidney, lung, muscle, adrenal, heart and skin. In liver and spleen Ad5CMV-directed EGFP expression was localized to reticuloendothelial system (RES) cells in contrast to microvessel restricted Ad5ROBO4 directed expression. Lung, liver, spleen and muscle maintained vector specific expression levels and patterns seen with the higher vp dose (FIG. 4).

### Example 6

This example illustrates that liver detargeting in Rag2KO mice abrogates promiscuous host organ EC Ad5ROBO4 reporter expression.

The ability to inhibit liver viral particle sequestration by warfarin-mediated blood coagulation factor depletion was used in hCAR(−) wild type mice (Waddington S N et. al. 2008; Alba R et al. 2010) to demonstrate target cell vector payload expression in the context of low hCAR expressing ECs. Liver detargeting efficiency was tested in our Rag2KO/KO mice. Warfarin pretreatment on day −3 and −1 before injection of 1×10¹¹ vp AdCMV-EGFP. revealed a diminution of hepalocyte reporter expression (FIG. 4). It was tested whether warfarin pretreatment in the absence of the hCAR transgene would produce host organ Ad5ROBO4 EC expression (FIG. 5) in RCC tumor-bearing mice. Compared to the hCAR mice, warfarin treatment either failed or barely produced detectable tissue reporter immunofluorescence in seven of nine host organs (FIG. 5A and data not shown (brain)). Similar to hCAR transgenic mice, there was focal, scattered liver and splenic EC expression in warfarin-treated, Ad5ROBO4 injected, Rag2KO/KO mice (FIG. 5A).

The discordance between immunofluorescence localization and intensity between host organs of hCAU transgenic compared to warfarin treated Rag2KO/KO mice, motivated a more quantitative analysis of Ad5ROBO4-mediated reporter gene expression. Host organ immunoblots revealed negligible expression in five of seven organs either with or without warfarin pretreatment. Warfarin produced a pronounced inversion of splenic versus hepatic reporter gene expression (FIG. 5B).

### Example 7

This example illustrates that Ad5ROBO4 EC targeting is maintained and differentially enhanced in both orthotopic and xenograft tumors compared to host organs following warfarin-liver detargeting.

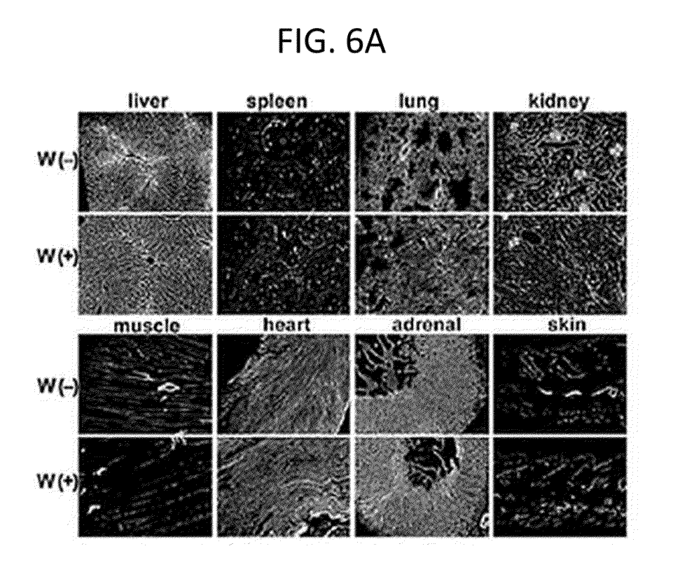

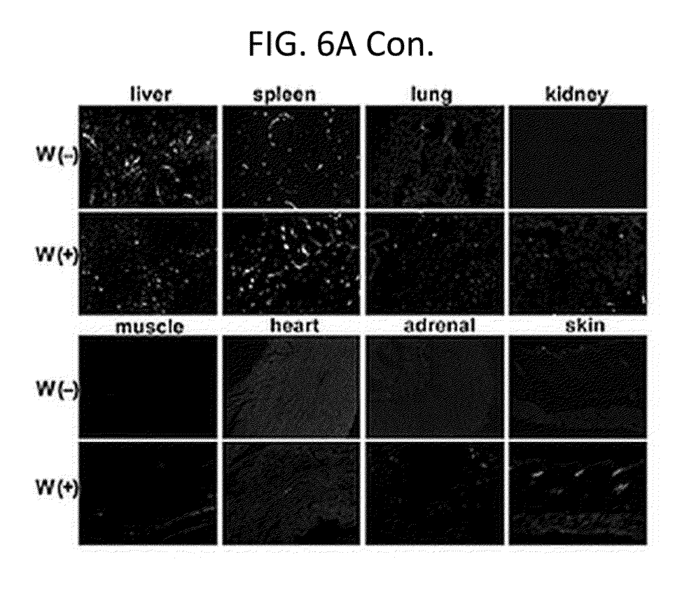

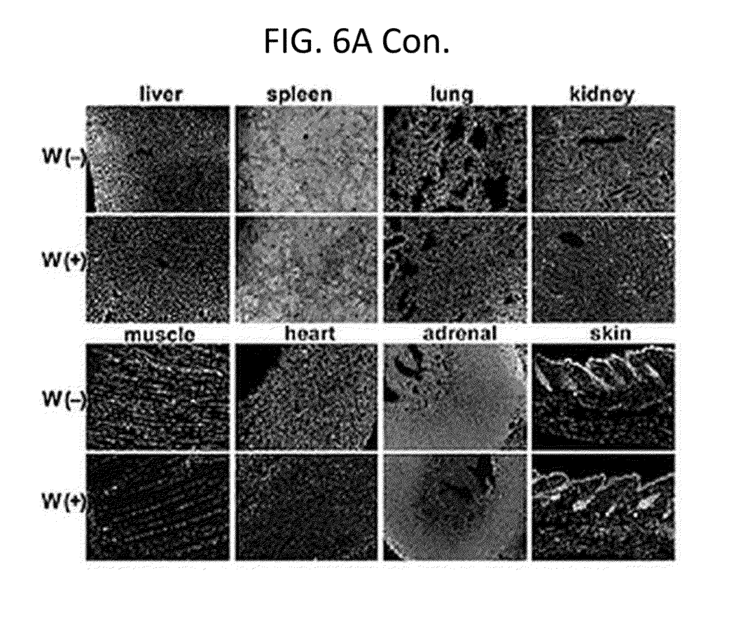

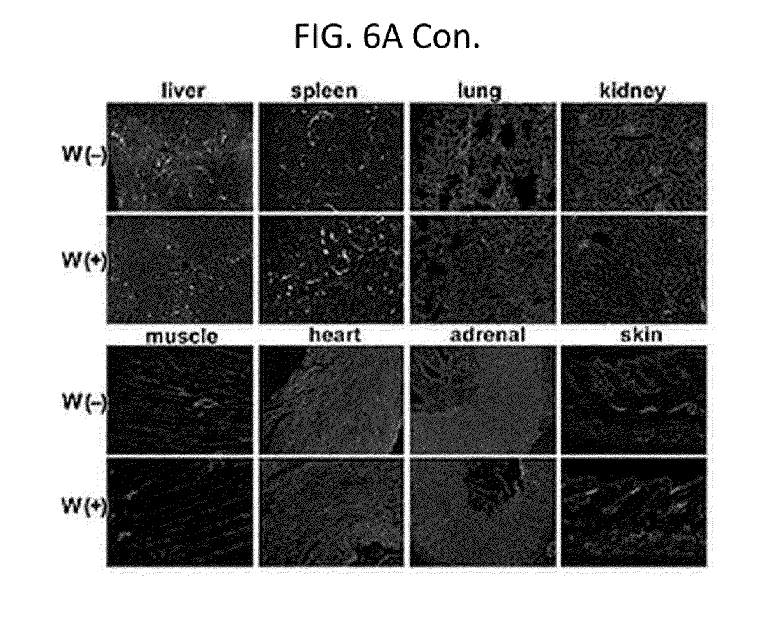

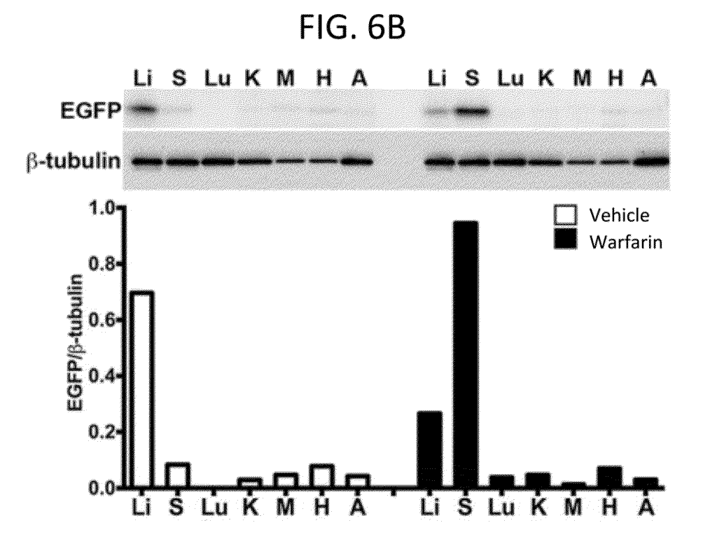

As liver detargeting effectively eliminated host organ Ad5ROBO4 EC expression, our next question was whether tumor EC expression would be similarly diminished. Consistent with baseline endogenous ROBO4 protein expression (FIG. 1) scattered EC reporter expression was detectable in both orthograft and xenograft RCC tumors even in vehicle-treated mice (FIG. 6). Warfarin pretreatment produced increased EC reporter expression at both tumor locales. Warfarin pretreatment increases splenic EGFP EC expression however all other organs except liver display sporadic or no reporter immunofluorescence. (FIG. 6) To quantity frequencies of immunofluorescent EC reporter gene expression in tumor versus host organs in the presence or absence of warfarin image analysis of tumor sections from 3-4 mice were used (FIG. 5). Warfarin produced an eight-fold increase the EGFP(+) computed to EC marker area in orthografts and a six-fold increase in xenografts (FIG. 5). Five of seven host organs evidenced minimal expression, albeit with single outlier mice in each organ. Warfarin produced a 1.7-fold decrease in hepatic and a 2.6-fold increase in splenic expression. Immunoblotting of liver extracts from Ad5ROBO4 injected mice (FIG. 6C) revealed a four-fold decrease of liver (EC localized. FIG. 6A) EGFP expression normalized to tubulin and a two-fold expression decrease normalized to VECadherin. In contrast tumor EGFP protein expression increased 1.4-fold at both sites following warfarin pretreatment. The splenic expression is markedly increased by warfarin whereas liver expression is decreased. (FIG. 6) Collectively, these data demonstrate the tumor EC selectivity of the Ad5ROBO4 vector.

### Example 8

This example illustrates Ad vector expression in ECs, generating active drug with secretion into the bone marrow.

In these experiments, an EC-specific vector configuration contained 3 kb of the human Magic Roundabout (ROBO4) enhancer promoter. ROBO4 is specifically expressed in ECs. It was confirmed that the EC specificity using an Ad5ROBO4-EGFP vector. This vector was expressed in tumor neovascular ECs, liver, spleen, and bone marrow sinusoidal ECs. Another vector configuration included Ad5ROBO4-EGEP with a bacterial cytosinc deaminase prodrug converting enzyme that can produce the cytotoxic chemotherapeutic, 5-fluorouracil (5FU) from 5-fluorocytosine (5-FC). EC-generated 5-FU ablated host bone marrow hematopoietic cells, The Ad vector configuration was exclusively expressed in ECs. generating active drug with secretion into the bone marrow microenvironment to achieve host cell killing.

### Example 9

This example illustrates mobilization of granulocytes, monocytes, and lymphocytes from the bone marrow to the peripheral circulation and the spleen with a Ad5ROBO4sCXCR42-23 vector.

In these experiments, an AdROBO4 vector configuration containing a transgene encoding a truncated CXCR4 receptor (an example of a “decoy receptor”) was constructed to affect angiocrine adjacent tissue modulation. This chemokine receptor exclusively binds and is activated by the chemokine stromal derived factor-1 (SDF1). The truncated transgene encodes an SDF1 “ligand trap” that is engineered to sequester SDF1 from CXCR4 expressing cells. Intravenous injection of this Ad5ROBO4sCXCR42-28 vector produced mobilization of granulocytes, monocytes, and lymphocytes from the bone marrow to the peripheral circulation and the spleen. These data are consistent with EC angiocrine secretion of sCXCR4 in the bone marrow, and breaking the attachment of CXCK4 hematopoietic progenitor cells from their CXCR4 mediated bone marrow niches.

### Example 10

This example illustrates selective targeting of ECs with an MBO-Ad5 vector configuration.

In these experiments, an Ad vector was created that can selectively target ECs via vector transduction. This vector was based on our discovery of “myeloid binding protein” (MBP) on the surface of myeloid cells that avidly bound to Ad vectors expressing phage peptide libraries inserted on the Ad vector fiber-knob. An Ad vector was created that was “deknobbed,” and contained a chimeric Ad5-T4 phage fibritin shaft and trimerization domain displaying the MBP peptide. In contrast to the MBP myeloid binding, the MBP-Ad5 vector selectively transduced ECs. Results included EC specific MBP-Ad5-EGFP expression in multiple host organ ECs including expression in brain ECs and expression within kidney ECs. The brain and kidney EC targeting have tremendous therapeutic implications for glioblastoma, Alzheimer's, multiple sclerosis, ALS, and for glomerulosclerosis, and interstitial renal nephritis.

### Example 11

This example illustrates the ability of MBP-Ad5 vectors to target specific tumor microenvironments using tumor-specific tuned promoters.

In these experiments, an Ad vector included tumor EG targeting with this MBP vector using the ROBO4 enhancer/promoter fragment. The EC specificity of the MBP-Ad vector was conferred via vector entry (transduction). Transgenes can act as “payloads” into the MBP-Ad vector, which contains DHA enhancer/promoter elements that are “tuned” to the tumor microenvironment, MBP-Ad vector configurations including “tumor tuned” promoters can transduce multiple host and tumor ECs, but solely expressed in tumors due to characteristics conveyed on their associated and embedded ECs. These tumor EC specific characteristics can include but are not limited to activation by hypoxia, DNA damage stress, endoplasmic reticulum/unfolded protein response stress, and redox/free radical stress. EC angiocrine engineering can tailor solely to the tumor microenvironment to enhance potency and specificity by arming MBP-Ad5 vectors with tumor-specific tuned promoters.

### Example 12

This example illustrates testing for PCA bone metastases growth inhibition due to dysregulation of CSC bone niche cellular components by angiocrine targeted prodrug-converting enzyme expressing Ad vector configurations.

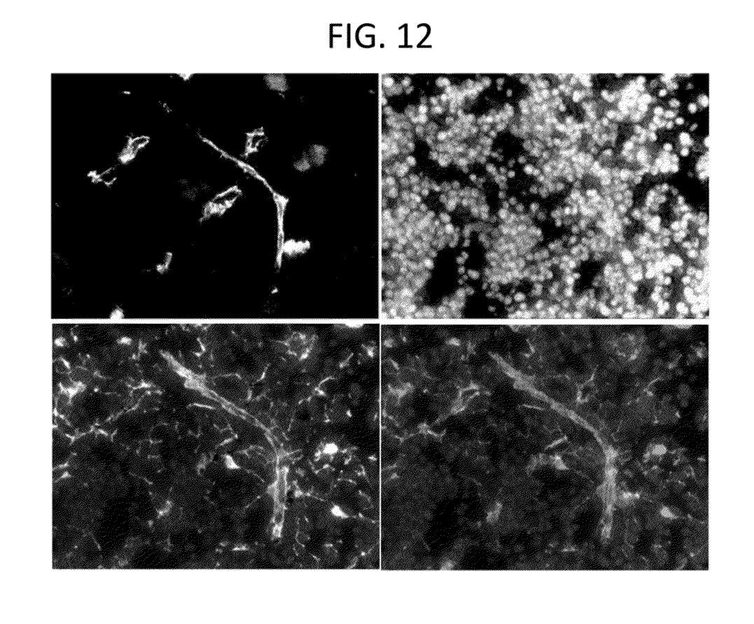

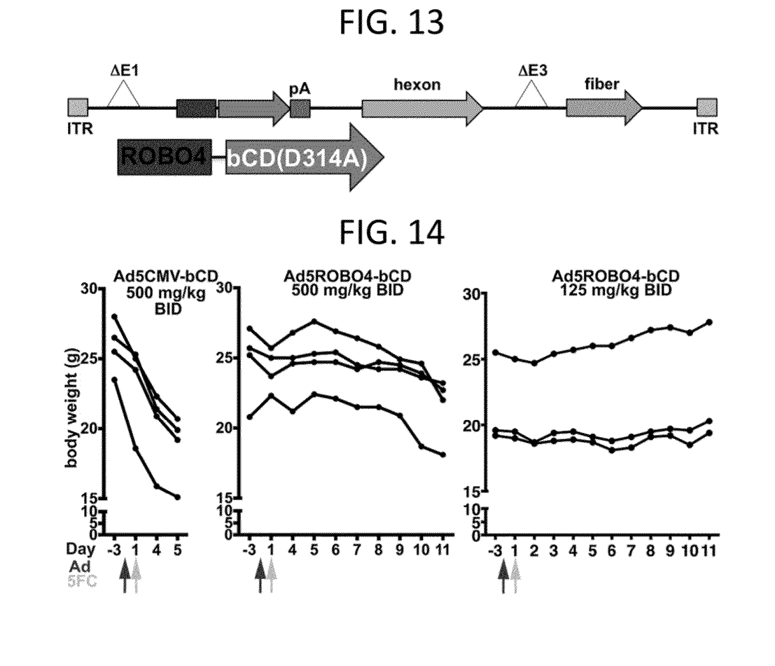

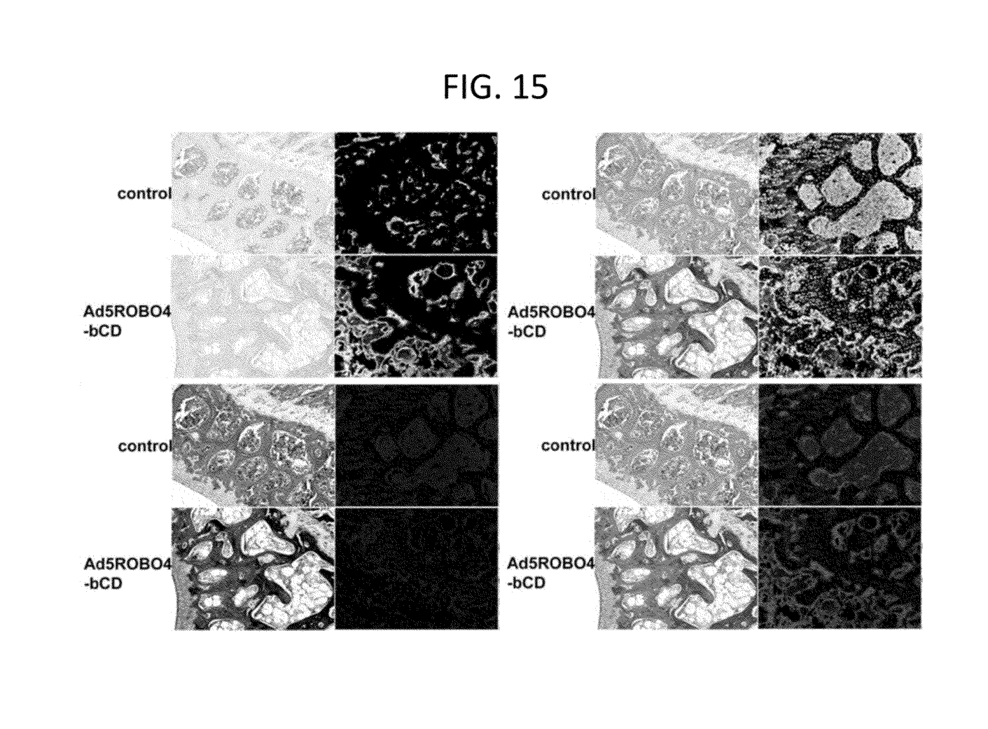

Host sinusoidal capillaries are principally composed of ECs, therefore the BM niche components can be particularly susceptible to angiocrine targeted Ad vector configurations. One example of the EC-niche cell spatial relationship is the localization of the principal SDF1 (CXCL12) producing BM niche component, the CXCL12 Abundant Reticular (CAR) cell (Omatsu et al. 2010; Greenbaum et. al. 2013). In these experiments, immunofluorescence was used to determine the EC-CAR spatial organization in the femur. The data demonstrate the investment of bone sinusoidal vascular ECs by CAR-EGFP cells (FIG. 12). Angiocrine-produced 5-FU (FIG. 13, FIG. 14, FIG. 15) can dysregulate the host bone marrow niche to effect PCA CSC depletion via niche eviction and quiescence abrogation. FIG. 13 illustrates an embodiment of an EC targeted prodrug-converting enzyme Ad vector Ad5ROBO4-bCDD314A. The bacterial cytosine deaminase (bCD) cDNA contains an aspartate-alanine substitution (D314A) enhancing 5-fluoroeytosine (5-FC) to 5-fluorouracil (5-FU) conversion. The principal RNA processing dysregulation mediated by 5-FU can enable functional disruption of quiescent bone niche components. There was an Ad valor expression gradient between intra and peritumoral BCs and distal uninvolved bone marrow (FIG. 10), that supports differential CSC versus HSPC bone niche targeting.

Focal intratumoral EC production of the stem cell ligand decoys can permit selective mobilization of PCA CSCs compared to host HSPCs. A collection of lineage-restricted transgenic reporter mice can be used to elucidate the distinct niche cell type targeted by angiocrine 5-FU production. Physical relationships between lineage-marked cells and metastatic PCA cells can be established and preferential sensitivities of niche cellular components and host HSPCs that are spatially dependent or independent of angiocrine 5-FU production can be tested. Differential PCA and niche lineage cell fluorophore marking can be used for frequency enumeration, quiescence, proliferation, and apoptosis analyses. Cell sorting can be used for candidate gene and unbiased expression profiling focusing on secreted and membrane-tethered molecules directing CSC-niche maintenance potentially dysregulated by angiocrine 5-FU production. Engineered PCA cells that can report on quiescence versus proliferation allow for the determination of the disruption extent of angiocrine 5-FU on niche CSC maintenance. Deployment of ECniche cell culture modeling (Seandel et al. 2008; Butler et al. 2010; Kobayashi et al. 2010) can allow further delineation of the mechanisms of angiocrine-CSC disruption.

Bone sinusoidal BCs can be exploited to produce and then secrete our prodrug product, 5-FU into the bone niche microenvironment. Focal 5-FU can differentially dysregulate host cellular niche components embedded within PCA metastases compared to uninvolved bone marrow regions. EC specificity and tumor bias was validated of the Ad5ROBO4 vector (FIGS. 8-10), and target vector Ad5ROBO4-bCDD314A embodiment was created (FIG. 13). bCDD314A is a bacterial derived cytosine deaminase containing an aspartate to alanine point mutation. bCDD314A possesses a marked increase in 5-FC-5FU conversion activity compared to wild type bacterial or yeast CD (Fuchita et al. 2009) (Duarte et al. 2012). An experiment with Ad5-bCDD314A IV injection in Rag2KO mice bearing 786-O ROC xenografts was performed. bCDD314A transgene activity of an Ad5CMV vector that is expressed in liver and spleen was tested. Despite warfarin-mediated liver detargeting (FIG. 5), Ad5CMVbCDD314A treated mice lost weight after 4 days of twice daily 5-FC, 500 mg/kg ip (FIG. 14). An embodiment Ad5ROBO4 vector doubled the 5-FC tolerance. Further dose reduction eliminated phenotypic toxicity. Warfarin on day −3/−1 and Ad vector injection on day 0. (FIG. 14) The induction of toxicity in the mice was in striking contrast to studies wherein IV AdCMV-bCD injection decreased hepatic colonic metastases growth while sparing host hepatocyte function (Topf et al. 1998). This discrepancy can be due to an enhanced potency of the bCDD314A transgene compared to the wild type counterpart. In these experiments, warfarin pretreated mice injected with Ad5ROBO4-bCDD314A appeared unaffected until weight loss starting on day 9 of 5-FC treatment (FIG. 14). At sacrifice on day 11, examination of the bone marrow revealed focal ablation of hematopoietic elements in the Ad5ROBO4-bCD injected mice (FIG. 15). At sacrifice on day 11, examination of the bone marrow revealed focal ablation of hematopoietic elements in the Ad5ROBO4-bCD injected mice. (FIG. 15) Analysis of RCC xenografts revealed apoptosis and necrosis in the vector-treated compared to untreated tumors. A dose reduction test was performed for a nontoxic 5-FC dose in nontumor bearing Rag2KO mice (FIG. 14). The results demonstrate that angiocrine bCD 5-FU production can be accomplished without host toxicity. These results support the in vivo functionality of the bCDD314A transgene, and that angiocrine targeted Ad vectors can affect the bone marrow microenvironment.

### Example 13

This example illustrates testing of angiocrine-targeted prodrug dysregulation of bone marrow niche supporting cell lineages.

In these experiments, a prioritized panel of lineage marked mice were interrogated (FIG. 16). Prioritization can be based on distance from bone marrow sinusoidal ECs. CAR cell frequencies and perivascular locale alterations can be tested and quantified for anatomic and morphological localization within metastatic tumors and uninvolved bone marrow using tissue section immunofluorescence image analysis and flow cytometry gated on GFP, CXCR4, and VCAM cell surface markers (Omalsu et al. 2010). CAR cell functional alteration can be tested by bone marrow SDF1 ELISA. CAR cells are the predominant SDF1 source (Omatsu et al. 2010) but other bone niche components, such as osteoblasts (OBs), mesenchymal and endothelial cells can additionally contribute to marrow SDF1 production (Greenbaum et al. 2013). To further test for 5-FU mediated CAR cell functional impairment, GFP flow sorted CAR cells can be cultured in adipogenic or mesenchymal media, the former to test adipocyte differentiation and the latter testing for colony-forming cell-fibroblast (CFC-F) generation (Omalsu et al. 2010; Greenbaum et al. 2013). These assays can provide mechanistic insight into how angiocrine-targeted 5-FU production alters CAR cell function. As nestin(+) cells also abut bone sinusoidal capillaries they can be used for lineage tracing (FIG. 16)(Nagasawa et al. 2011). Prx1 is a marker of mesenchymal progenitors/stem cells (MSCs) (Logan et al. 2002). Prx1 cells are also requisite niche components (Ding and Morrison 2013; Greenbaum et al. 2013). Moreover, recent data suggest that MSCs contribute to bone metastatic progression in general (Kob and Kang 2012), and are an additional source of SDF1/CXCL12 production in particular (Ye et al. 2012: Borghese et al. 2013; Mognetti et al 2013). Osteoblasts (OBs) have also been suggested as crucial PCA/CSC niche components (Chung et al. 2009; Schulze et al. 2010; Fritz et al. 2011; Schulze et al. 2012). Angiocrine-5-FU effects on OBs will be determined using Col2.3-GFP lineage tracing (FIG. 16). While OBs are not as intimately associated with PCs as CAR cells, a sinusoidal capillary subset closely approximates the OB-enriched bone surface and as such, could be impact this endosteal niche. Flow-sorted Prx 1/MSC and Col2.3./OB GFP(+) cells from 5-FC treated mice can be tested for differentiation and bone formation perturbation in cell culture assays (Weilbaecher and Novack LOSs) as described above for CAR cells (Su et al. 2012; Yang et al. 2013).

A corollary to angiocrine 5-FU niche deregulation is perturbation of PCA CSC maintenance, abrogating CSC quiescence eventuating in CSC depletion and proliferative transit amplifying cell population expansion. Multiparameter immunofluorescence can be engaged using PCA CSC and HSC stem and differentiation markers in both tissue sections and flow cytometry. Approaches to functionally report on CSC and HSC quiescence and proliferation can also be used. Stein cell quiescence detection data can be used from on bromodeoxyuridine label retention.

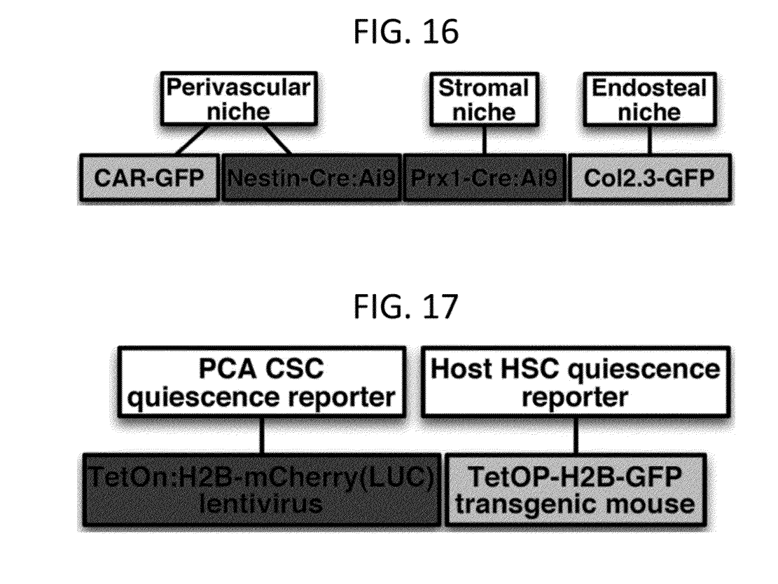

Dilution of a chromatin-binding histone 2B-GFP (H2B-GFP) fusion protein can be used to estimate CSC quiescence (Kanda et al. 1998: Hadjantonakis and Papaioannou 2004; Wilson et al. 2008). Labeling can be performed using different H2B-fluorophore colors to assay both populations in the same mouse (Hadjantonakis et al. 2003) (FIG. 17). A lentiviral dual rtTA/TRE “tight” TetON-histone 2B (H2B)-mCherry virus can be constructed like the TetOff system (Falkowska-Hansen et al. 2010). IGR-CaP1 cells can be lentivirally infected with TetON-H2B-mCherry and CMV-pLUC and select DOX induced reporters and constitutive LUC expression.

TetOP-H2B-GFP mice bitransgenic can be obtained for both the rtTA TetON operator and TREH2B-GFP transgenes (Foudi et al. 2009) (JAX) and intercross with Rag2KO mice. DOX-pre-induced IGRCaP1: TetON-H2B-mCherry cells can be intracardiac injected into DOX pretreated TetON-H2B-GFP:Rag2KO mice (FIG. 17). The six-eight week lag time for IGR-CaP1 gross bone metastases development can allow for a DOX withdrawal washout period to test for H2B label retention consistent with stem and early progenitor cells. PCA CSC versus HSC quiescence can be quantified by tissue and flow cytometric enumeration of ted (CSC) and green (HSPC) fluorescence. Additional testing for differential HSC mobilization and repopulation capability can be performed.

Conventional bone tissue section and flow cytometric immunofluorescence can be used to interrogate changes in the metastatic tumor and the bone marrow niche cellular composition. Tissue PCA versus host cellular areas can be tested for proliferation, cell death, and EC vascular marker immunofluorescence. To facilitate PCA bone localization, add to proliferation/apoptosis and flow cytometry enumerations a constitutive IGR-CaP1:CMV-H2B-mCherry:LUC (Addgene) cell line can be created. Alterations in PCA CSC versus PCA progenitor or more differentiated PCA cells can be determined by CD133, CD44, EpCAM, CD49f, CK5 and CK8 immunofluorescence co-localized with IGR-CaP1:H2B-mCherry expression, PCA hierarchical composition can be more precisely quantified by flow cytometry. Dissociated bone tumors can be gated on mCherry, the epithelial identity of those gated cells confirmed by EpCAM, then subfractionated based on CK5 (basal) versus CK8 luminal, then further fractionated based on CD44 and CD49f. EGFP/EpCAM:CD44highCD49f:CK5high;CD8low can be presumed to be stem cells. HSC/HPC frequencies can be screened using the KLS/CD150+/CD48−/FLK-panel (Mayle et al. 2013). The inverse marker distribution can be designated luminal cells. To investigate whether our EC targeted Ad vector is truly affecting an angiocrine rather than a systemic phenotype, metastatic tumor, BLI-identified uninvolved bone marrow, blood, and multiple host organs can be tested for 5-FU levels by HPLC (Kievit et al. 2000).

### Example 14

This example illustrates cell culture experiments to test PCA stem cell potential. PCA stem cell potential can be functionally tested. 5-FU can decrease the frequency of intratumoral CSCs and can impair CSC renewal function. Prostaspheres are considered one hallmark of PCA CSC capacity (Azuma et al. 2005; Guo et al. 2012). Prostasphere renewal capacity can be tested using serial culture. Ability to generate proliferative progeny can be tested by scoring prostasphere size attainment. CSC renewal capacity can be tested using serial limited dilution and serial transplantation experiments (Qin et al. 2012). In addition to these stem cell potential assays, solo and co-culture experiments can also be engaged testing for 5-FU-mediated changes in EC-tumor or EC-tumor-osteoblast cross talk. To create an EC cell culture niche, the Ad5E4-ORF1 transfected HUVEC model can be used (Seandel et al. 2008). These E4-ORF1 HUVECs can be maintained for multiple passages, and support HSCs for prolonged cell culture periods (Kobayashi et al. 2010). To test for angiocrine functional alterations, 5-FC treated E4-ORF1 transfected HUVECs infected with AdROBO4-bCD or control vectors can be interrogated for differential growth factor and chemo/cytokine secretion using commercial proteomic antibody arrays. Array data will be validated by Western blotting and ELISAs. Tumor-EC co-cultures can be established by “parachuting” IGR-CaP1 tumor cells onto E4-ORF-EC cord lattices. Tumor and ECs can be prelabeled with different fluorescent dyes and global gene expression and proteomic secretion alterations profited from FACS sorted populations.

Data can be produced from at least 4-6 mice injected with experimental, and 4-6 mice injected with control Ad vectors for statistical analysis. Tissue cellular EGFP expression frequencies can determined by measuring the EC-colocalized EGFP positive area compared to total section area. These area ratios can be obtained from the average of 4 sections per mouse. Cell culture experiments can be repeated 4-6 times as can limit dilution tumor formation analysis. Statistical significance testing can use the non-parametric Mann-Whitney U test, and one-way ANOVA.

### Example 15

This example illustrates testing for PCA CSC versus host HSPC mobilization, niche depletion, and cytotoxic chemotherapy enhancement mediated by angiocrine targeted Ad vectors expressing stem cell ligand decoys.

PCA CSCs can be regulated by several stem cell receptor/ligand signaling modules, including CXCR4/CXCL12 (SDF1) (Sun et al. 2005; Shiozawa et al. 2011; Dubrovska et al. 2012), NOTCH/Jagged/Delta (Leong and Gao 2008; Wang et al. 2010; Ye et al. 2012), and WNT/Frizzled (Horvath et al. 2007; Schweizer et al. 2008; Kawano et al. 2009; Takebe et al. 2011). The CXCR4-SDF1 axis can be targeted. CXCR4-SDF1 decoy data can be used as a template for testing of angiocrine Ad vectors slated for NOTCH or WNT ligand decoy signaling disruption. Our data revealed peritumoral EC Ad vector expression with a gradient diminution in distal metastasis-bearing bone (FIG. 10). Intra- and peri-tumoral EC ROBO4 promoter activation can produce focused CSC mobilization while preserving retention of host HSPCs in uninvolved bone regions. There is extensive expression of the EC-targeted Ad vector within and adjacent to IGR-CaP1 bone metastases. The Ad vector expression gradient between intra and peritumoral ECs and distal uninvolved bone marrow is support for differential CSC bone niche targeting. Intra- and peri-tumoral EC ROBO4 promoter activation could produce focused CSC mobilization while preserving retention of host HSPCs in uninvolved bone regions (FIG. 10).

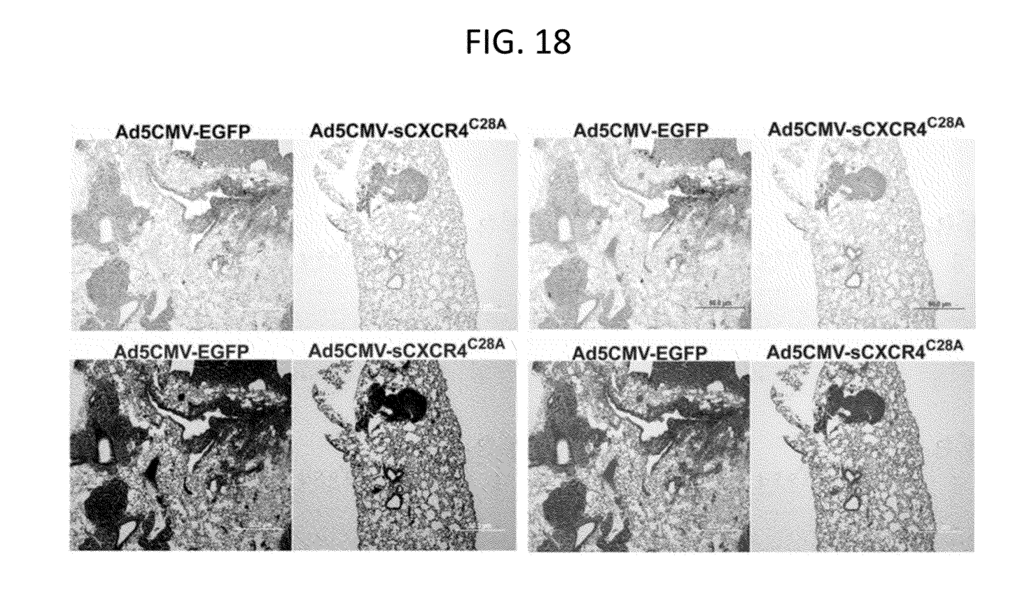

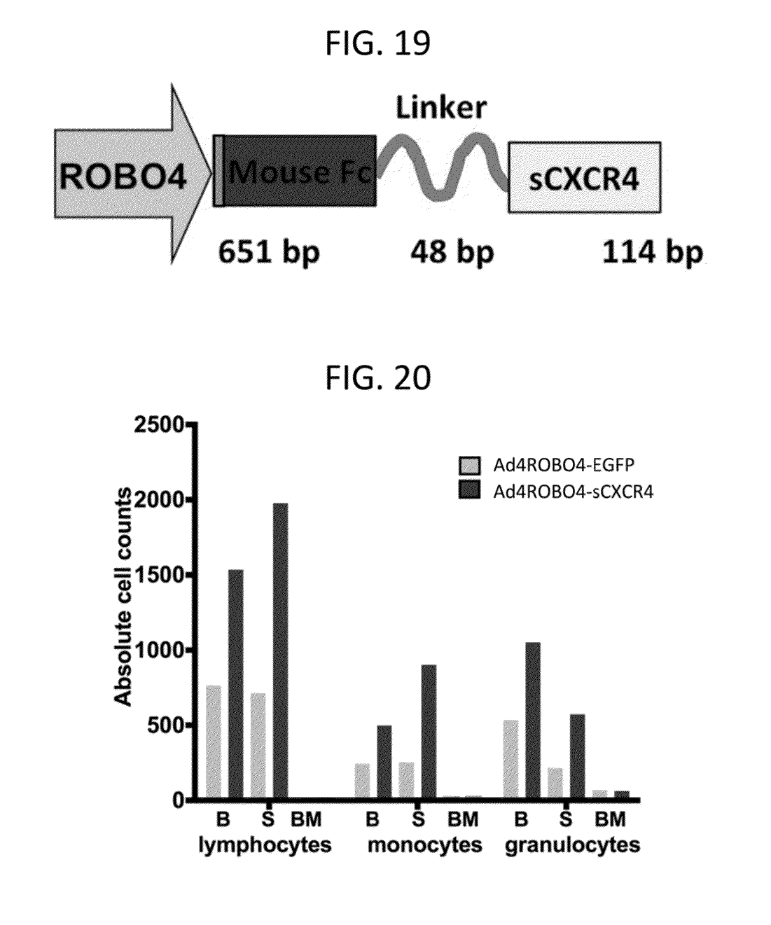

A soluble, truncated “sCXCR4” expressing Ad vector was created. A Ad5CMV-sCXCR4-Fc was constructed and activity tested (sec FIG. 14 for ROBO4 vector). The vector transgene encodes amino acids 2-28 of human CXCR, which is the SDF1 ligand binding domain, fused to a mouse immunoglobin heavy chain (Fc) fragment. The vector was validated for mammalian cell expression following virus infection in cell culture and in the plasma of tail vein injected mice. Systemic CMV-sCXCR4 vector injection inhibited B16/F10 mouse melanoma lung metastatic implantation and growth (FIG. 18). Systemic CMV-sCXCR4 vector injection inhibited B16/F10 mouse melanoma lung metastatic implantation and growth. (FIG. 18). TheFC targeted Ad5ROBO4-sCXCR4-Fc vector embodiment (FIG. 19) was created and tested. The insert contains the cDNA encoding lite CXCR4-SDF1 ligand-binding domain linked lo mouse immunoglobin heavy chain for secretion and stabilization. (FIG. 19)

In these experiments, warfarin-pretreated, nonlumor bearing. C57B16/J and Rag2KO mice were IV injected with Ad5ROBO4-sCXCR4-Fc and a control Ad5ROBO4-EGFP vector. Flow cytometry demonstrated elevations of granulocytes, monocytes, and lymphocytes (likely bone resident B-Iymphocytcs) in blood (B) and spleen (S) of Ad5ROBO4-sCXCR4 injected mice (FIG. 20). Our results showed expression of Ad5ROBO4 in bone marrow ECs. The results suggest Ad vector hijacking of EC angiocrine functions mobilize cells from the bone marrow. The BM Ad vector expression gradient (FIG. 10) can focus and differentially amplify CXCL12 sequestration intra and perimetastatically. Our 1×10¹¹ viral particle dose has the dynamic range enabling ample decremental dose titration, to achieve selective CSC mobilization.

### Example 16

This example illustrated testing angiocrine-targeted stem cell ligand sequestration mediated dysregulation of CSC bone marrow niche retention.

Testing can be performed to determine that angiocrine stem cell ligand sequestration can differentially mobilize and deplete CSCs vs HSPCs, that the angiocrine-mediated CSC mobilization can affect loss of CSC compared to HSC quiescence and that angiocrine-mediated CSC mobilization can enhance sensitivity of PCA metastatic growth to docetaxel.

To lest for CSC mobilization, mice with BL1-verified PCA bone metastases can be IV-injected with Ad5ROBO4-sCXCR4. Blood can be analyzed by human Alu RT-PCR (Shiozawa et al 2011). If positive, blood PCA cells can be further enumerated by histone 2B(H2B)-mCherry flow cytometry (Shiozawa et al. 2011; Qin et al. 2012). If mCherry labeled cells are detected at sufficient frequency in whole blood, further enumeration of CSCs using our battery of stem cell markers can be performed. Companion bone marrow (BM) analyses can test for PCA CSC diminution by (low cytometry of H2B-mCherry-gated single cell suspensions of bone metastases additionally stained for PCA CSC stem cell matters. Potential shifting of metastatic PCA quiescence to enhanced proliferation can be initially determined by Ki67 flow cytometry. “Uninvolved” bones suggested by BLI can also be tested for PCA CSC multiplicity and quiescence/proliferation shitting by CSC stem marker and Ki67 whole BM analyses. Blood (PCA marker) and bone marrow (PCA-CSC markers) markers can be used as enumerations as benchmarks for decremental vector dose titrations if necessary to achieve differential CSC versus HSPC niche mobilization. To test for vector host BM mobilization elevations of hematopoietic elements in blood and spleen can be tested. To pinpoint HSPC versus differentiated cell mobilization, colony forming unit-cell (CFC-C) potential in whole blood can be tested. Further HSPC analyses can enumerate BM HSCs and HPCs using SLAM markers (Kiel et al. 2005; Mayle et al. 2013). The HSC population can be functionally characterized for Ad-sCXCR4 vector mediated long-term versus short term, LT-HSC and ST-HSC, frequencies in mouse reconstitution assays (Greenbaum et al. 2013; Mayle et al. 2013). These can be benchmark data from which CSC vs HSPC decremental vector dose titration is evaluated. Flow cytometric analyses of CSC vs HSPC BM mobilization and depletion can be further analyzed using multiparameter tissue immunofluorescence for proliferation markers, BrdU, Ki67, and a panel of CSC and HSPC markers. Differentiation of PCA versus host bone marrow elements can be facilitated by H2B immunofluorescence that produces a intense signal with low background (Hadjantonakis and Papaioannou 2004).

To test for loss of CSC quiescence mediated by our Ad vector stem cell ligand decoys, the dual color H2B washout experimental strategy can be engaged as detailed in (FIG. 17). DOX-pretreated PCA cells containing the TetON-H2B-mCherry-LUC virus can be injected into DOX-pretreated TetOP-H2B-GFP:Rag2KO recipients (Foudi et al. 2009; Falkowska-Hansen et al. 2010). Differential retention of BM niche green/red fluorescence following 6-8 wk washout can be enumerated by flow cytometry and tissue immunofluorescence, bolstered by additional markers (Foudi et al. 2009). Tumor progression can be tested using BLI in a group of mice following vector injection, and extended duration experiments testing for overall survival. The question can be addressed of differential CSC specific mobilization mediated by the sCXCR4 vector compared with the “gold standard” CXCR4 small molecule inhibitor, AMD3100. Each vector experiment can include an AMD3100 Alzet pump control emulating continuous vector-mediated sCXCR4 production.

Ad5ROBO4 vectors can be constructed and cell culture validated containing soluble NOTCH and WNT ligand decoys (sNOTCH and soluble Frizzle Related Protein (sFRP) receptors (Funahashi et al. 2008; Lavergne et al. 2011). The existence of NOTCH and WNT pathway cDNA and transgenic reporter mice can expedite efficacy screening and if necessary decremental Ad vector dose titration both in cell culture and in intact mice. Additional experiments can use the sCXCR4 experimental template, testing the degree of differential CSC versus host stem cell mobilization, CSC depletion, and potential PCA tumor growth inhibition achieved with the sNOTCH and sERP vectors. With the sCXCR4, sNOTCH, and sFRP data the vector can be selected with greatest CSC functional efficacy to carry forward for additivity testing with cytotoxic chemotherapy. Following the leukemia paradigm (Nervi et al. 2009; Essers and Trumpp 2010), sequential Ad-sCXCR4 or control Ad-EUC vector can be combined with “standard of care” docetaxel chemotherapy (Seruga and Tannock 2011). Additional data is available for IGR-CaPI cell docetaxel sensitivity. Docetaxel can be given for 2-4 weeks alter Ad vector injection. Tumor growth inhibition or regression can be followed by BLI. Blood can be serially sampled for PCA, CS, and HSPC frequencies as detailed for Ad-sCXCR4. BM and spleen can be analyzed for PCA CSC, HSPC frequencies using flow cytometry and tissue histopathology; proliferation, apoptosis, and vascularity can be tested using multi-marker tissue immunofluorescence bolstered by Western blotting. To further test for Ad-stem cell ligand decoy CSC niche eviction and consequent depletion cell culture experiments can be used. CSC abundance can be interrogated by the comparative quantity of prostaspheres formed from Ad-stem cell ligand decoy versus control vector injected mice. CSC renewal capacity can be tested by serial culture. Limited dilution single and serial tumor transplantation experiments can further investigate CSC numbers and functional capacity (Qin et al. 2012).

The angiocrine-targeted Ad vector strategy can be differentially localized in metastatic rather than uninvolved bone (FIG. 10). Thus, the Ad vector embodiments can focus CXCR4 blockade to tumor specific, rather than global bone marrow niches. Focal Ad vector-mediated sCXCR4 expression can selectively or preferentially affect CSCs rather than host HSCs/HPCs. The Ad vector system is tunable in regards to promoter selection. Dose titration, or vector switching to our EC tropic MBP vector that can contain enhancer-promoters with greater tumor microenvironment responsiveness, can achieve a specificity level exceeding global small molecule therapies.

The angiocrine Ad vector approach is also poly-ligand targeting. This targeting is relevant to CSC-niche crosstalk, as multiple ligand/receptor modules can to control PCA CSC maintenance (Karhadkar et al. 2004; Chang et al. 2011; Valdez et al. 2012; Ye et al. 2012). 1X1 ligand decoy combinations, or decoy collections collectively as single vector polycistronic combinations can be tested. Switchable promoter elements can be introduced within high capacity “gutless” vectors. The “theranostic” attractions of gutless vectors can be further tested.

### Example 17

This example illustrates testing theranostic polycistronic “gutless” Ad vectors for bone metastatic therapeutic and imaging efficacies.

Polycistronic vectors are emerging as enticing tools for regulation of complex biological processes. Premature nascent peptide release from the ribosome mediated by viral 2A peptide sequences allows for 1:1 expression of tandem cDNAs (Szymczak-Workman et al. 2012). There are 2A peptide sequences from several viral species that are used in polycistronic vectors. Rules for their sequence ordering within the vector have been established (Szymczak-Workman et al. 2012).

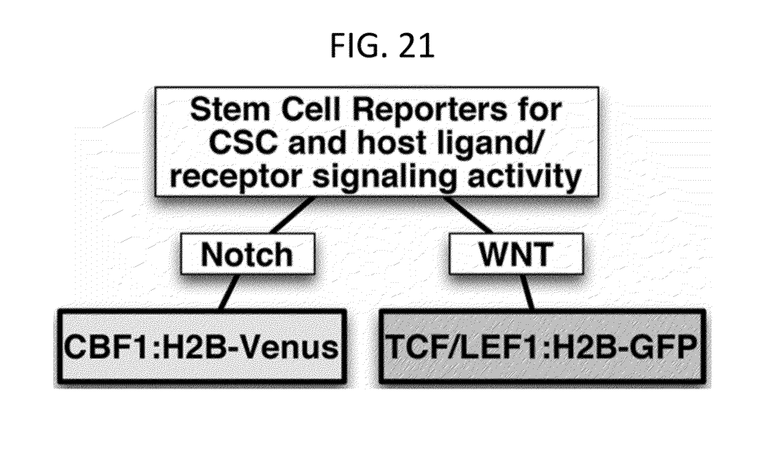

Previous work has demonstrated that polycistronic vectors can rescue quadra-T-cell receptor subunit knockout mice (Szymczak et al. 2004) and reprogram iPS cells (Carey et al. 2009; Shao et al. 2009). Polycistronic vectors have been used in first generation Ad vectors, and can be used for high capacity “gutless” vectors with their 37 kb capacity (Stadtfeld et al. 200S). Switchable control of gene products can be implemented. Switchable control can be applicable to SDF1-CXCR4 blockade wherein prolonged blockade produced paradoxical bone metastatic tumor growth enhancement due to osleoclastogenesis stimulation (Hirbe et al. 2007). Gutless vectors can achieve a theranostic agent switchable control to allow for cyclical therapeutics when disease recurrence is vector detected. Single, 1X1 vector combinations of our ligand decoys can be engaged. Combinatorial transgenic mouse and infectable/transfectable NOTCH and WNT reporters for Ad vector-mediated pathway signaling downregulation can be engaged (FIG. 21). Combinatorial transgenic mouse and infectable/transfectable NOTCH and WNT reporters for Ad vector-mediated pathway signaling downregulation. (FIG. 21) The CBF-H2B-Venus and the TCF/LEF-H2B-GFP constructs report on NOTCH and WNT respectively (Ferrer-Vaquer et al. 2010; Nowotschin et al. 2013). The cDNAs used for transgenic mouse construction are available from Addgene. IGR-CaP1 cells can be infected with these constructs and used for generation of bone metastases. Flow cytometric analyses of reporter fluorescence intensity can be used to determine the efficacy of Ad vector mediated NOTCH or WNT pathway downregulation (FIG. 21). To test for comparative Ad vector-mediated host pathway downregulation each transgenic mouse reporter (JAX available) can be obtained and multiparameter BM flow cytometry abstained with SLAM and lineage markers can be used.

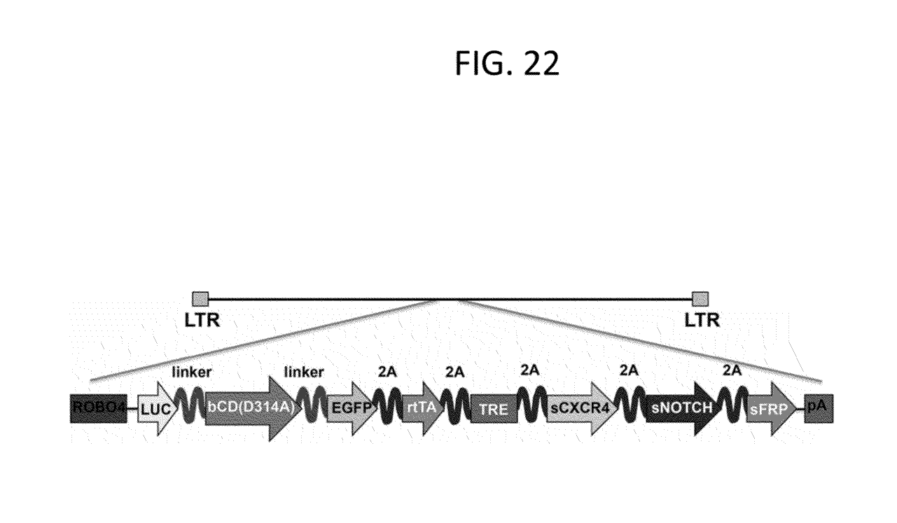

Polycistronic vector configurations can be constructed and tested. An embodiment of this vector is presented herein (FIG. 22). Design features of this vector can include but are not limited to polycistron EC-targeting via the ROBO4 enhancer promoter, constitutive expression of LUC for BL1 bone metastases growth, inhibition, or recurrence detection and FGFP for enhanced tissue immunofluorescence localization, constitutive prodrug converting enzyme expression that is functionally conditional due to prodrug dependence, and/or switchable doxycycline control of multiple stem cell ligand decoys (Xiong et al. 2006). Design features of this vector embodiment can include: 1) polycistron EC-targeting via the ROBO4 enhancer promoter. 2) Constitutive expression of LUC for BL1 bone metastases growth, inhibition, or recurrence detection and EGFP for enhanced tissue immunofluorescence localization, 3) Constitutive prodrug converting enzyme expression that is functionally conditional due to prodrug dependence, and/or 4) Switchablc doxycycline control of multiple stem cell ligand decoys.

Testing for CSC/HSC/HPC mobilization can be implemented. Combinatorial 5-FC:5-FU generation with multi-ligand mediated CSC niche eviction can be tested for metastatic growth inhibition efficacy compared to solo Ad-bCD vector data. Vector pretumor injection treatment can allow us to perform tumor dormancy and established tumor experiments using a single experimental design. Experimental duration can be extended and sequential vector polycistron expression activation performed on recurrent tumors of selected sizes. Imaging experiments can test metastatic tumor burden detection thresholds. To probe translational relevance our vectors can be tested for prolonged expression in syngeneic bone metastatic models. Additional viral capsid genetic and possibly chemical engineering can also be engaged obviating the anti-coagulant factor and producing immune evasion. Gene fusion strategies, viral species/type of 2A peptide and cDNA cassette polycistron ordering can all be altered to achieve a polycistronic vector requisite for bone metastatic efficacy. The number of cistrons can be reduced to achieve a functional encapsidated vector.

### Example 18

This example illustrates that MBP pseudotyping attenuated hepatocyte vector expression while producing widespread multi-organ vascular EC expression.

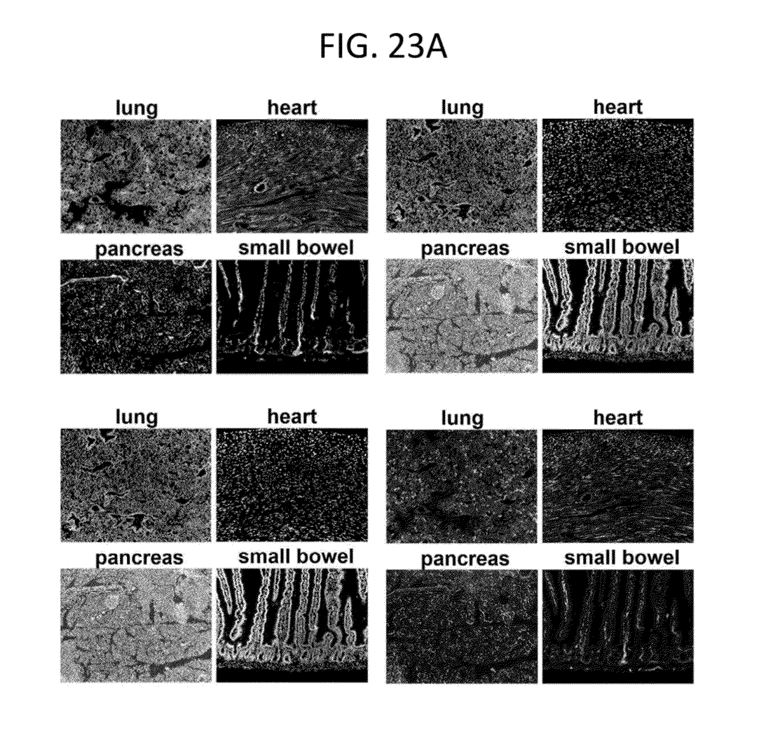

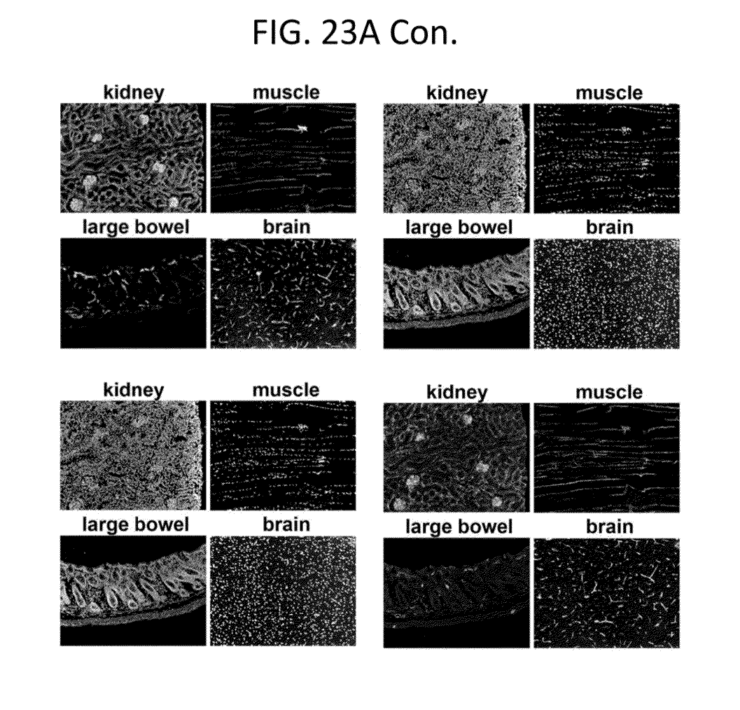

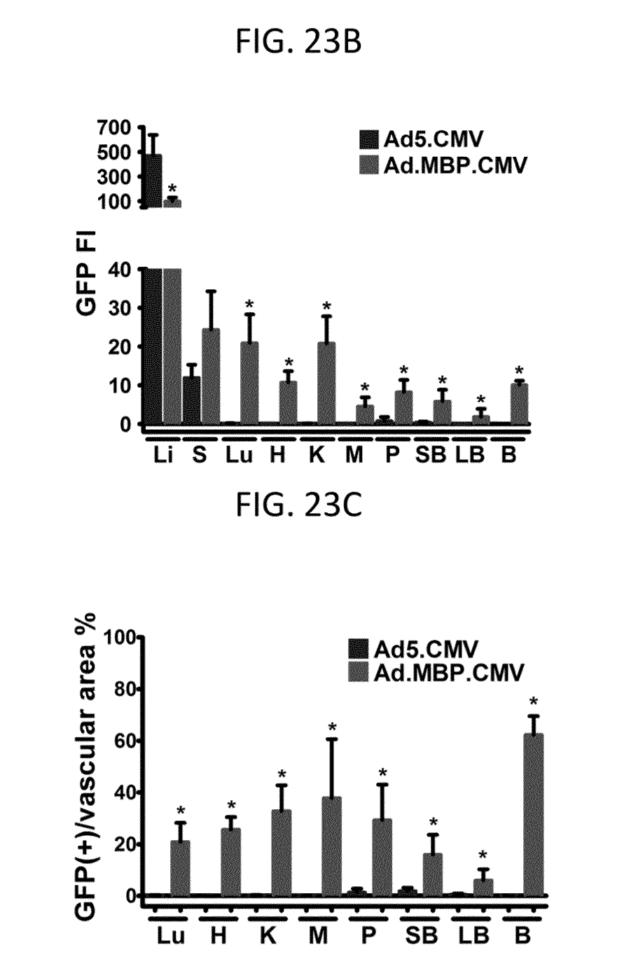

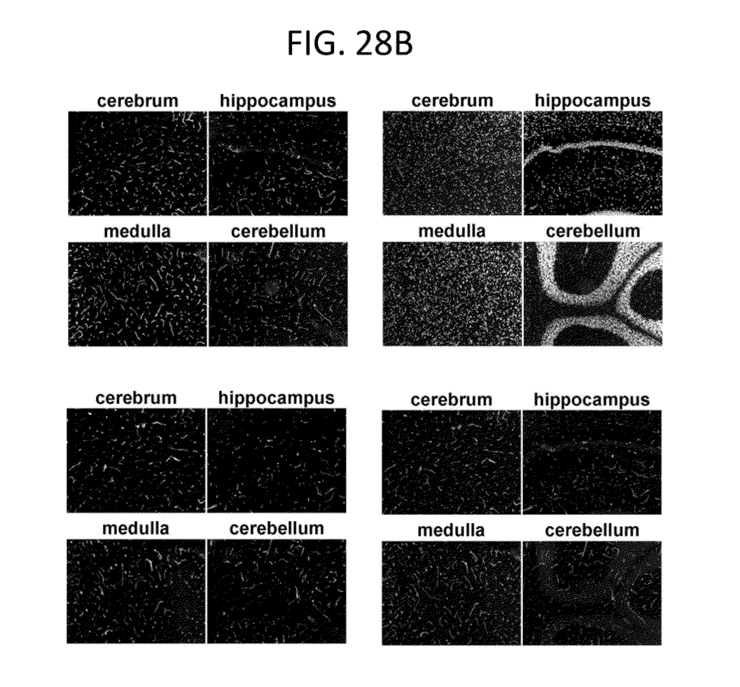

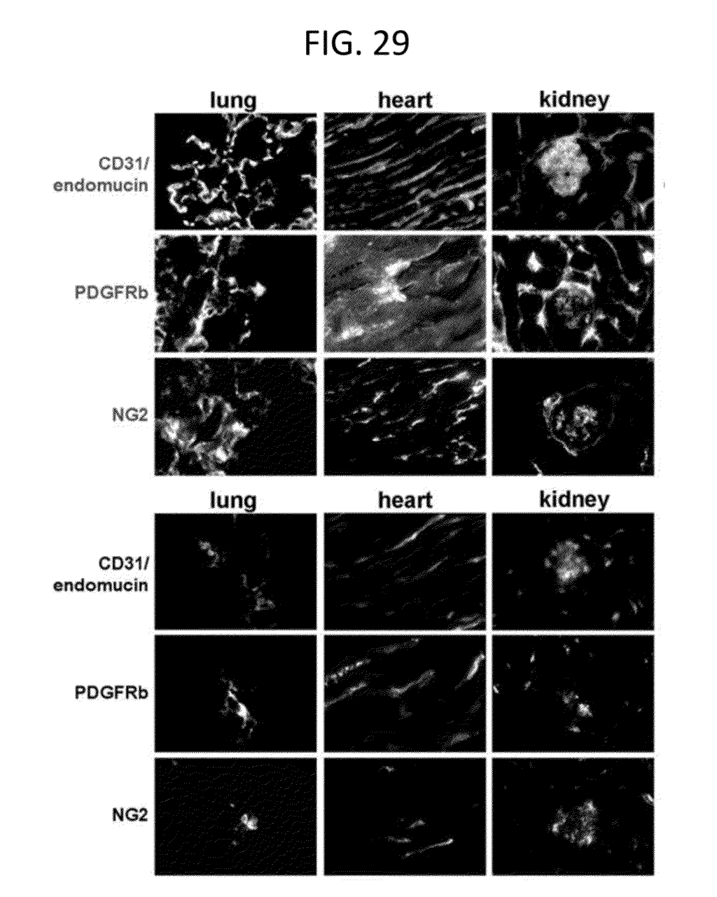

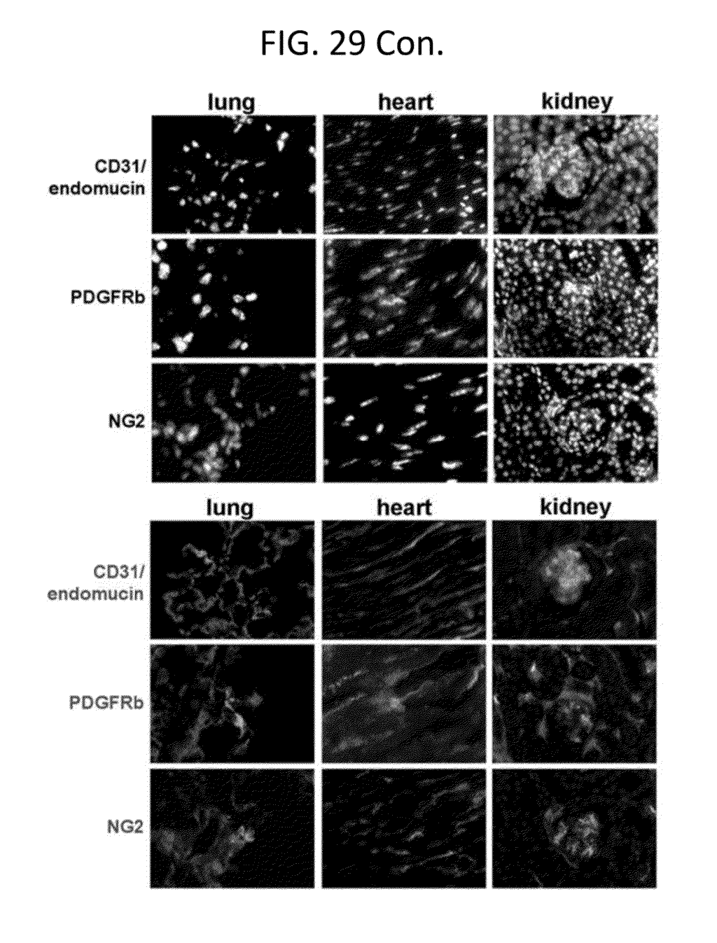

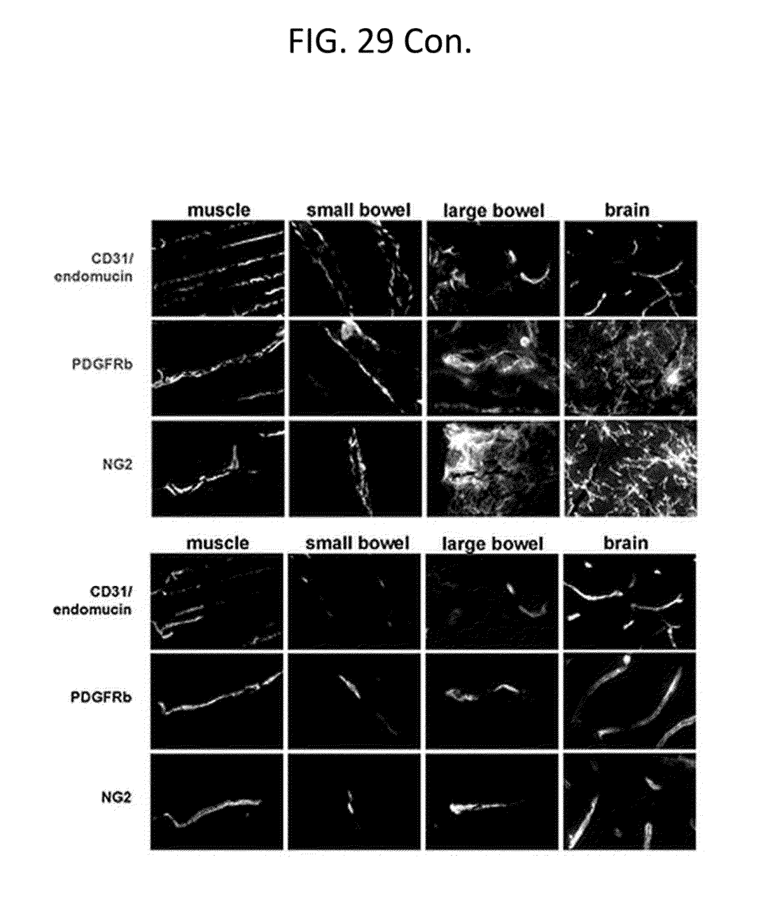

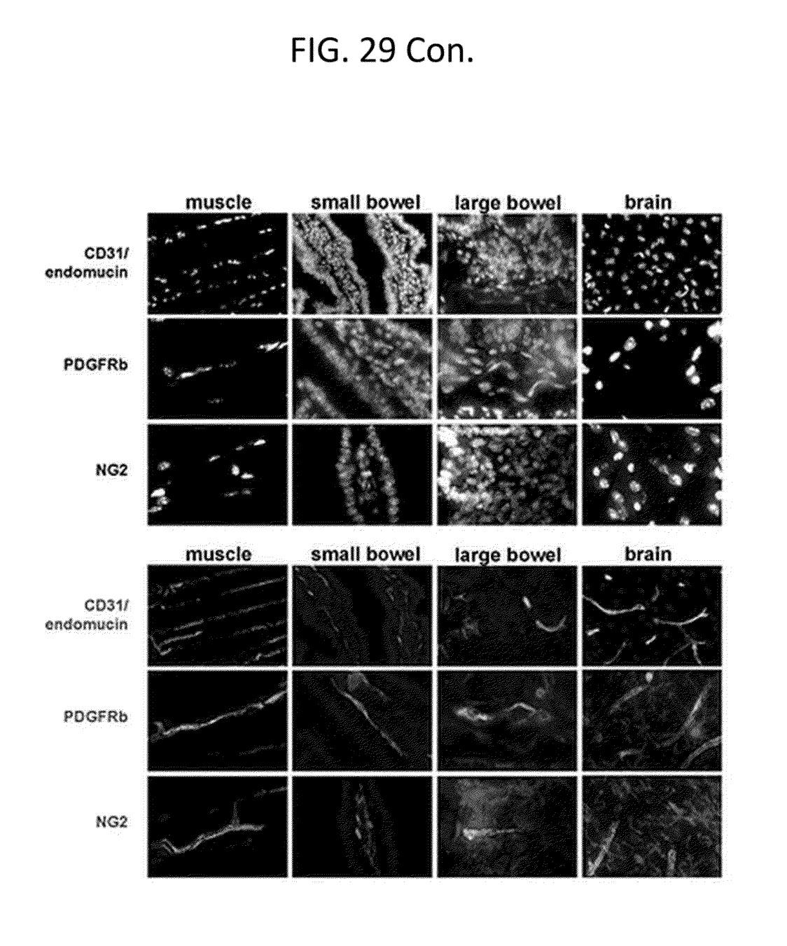

The multiorgan biodistribution of Ad.MBP.CMV was tested using semiquantitative tissue section immunofluorescence analysis. Ad5.CMV-mediated expression was predominantly localized in liver hepatocytes and detectable in reticuloendothelial system and endothelial cells (ECs) of spleen (FIG. 28A). Vector expression was scarcely found in lung, heart, kidney, gastrocnemius muscle, pancreas, small bowel, large bowel, and not detectable in any part of the brain (FIG. 23B). In contrast, Ad.MBP.CMV produced EC expression throughout the microvasculature of heart, kidney, muscle, pancreas, intestine, and brain (FIG. 23A). Vector EC co-localization was confirmed using high-magnification EFI imaging in these organs (FIG. 29). Surprisingly, robust transgene expression was detectable in ECs within tested brain regions including cerebrum, cerebellum, hippocampus, and medulla (FIG. 288). To quantify vector transgene expression, EGFP fluorescence intensity was summed in a tissue region of interest (ROI) and normalized by the ROI area (per μm²) in each organ. Liver sections from Ad.MBP.CMV-injected mice exhibited a 5-fold reduction in the EGFP fluorescence intensity compared with the Ad5.CMV counterparts (FIG. 23B). Liver detargeting was associated with 2-fold increase in vector expression in splenic reticuloendothelial cells and ECs (FIG. 23B). As lung, heart, kidney, pancreas, small and large bowel, and brain were eithervbarely or not transducible by the Ad5.CMV, the retargeting enhancement of the Ad.MBP.CMV to these organs was ranged from greater than 10-fold increase in pancreas, small bowel, and large bowel, greater than 100-fold increase in lung and kidney, greater than 1,000-fold increase in heart and muscle, and greater than 10,000-fold increase in brain (FIG. 23B, red bars versus blue bars for Lu, H, K, M, P, SB, LB, and B).

FIG. 28 illustrates incorporation of MBP into Ad5 detargeted the virus from liver hepatocyles, modestly increased gene expression in splenic marginal zone, and markedly enhanced gene expression in all regions of the brain. (A) EGFP expression in liver and spleen following intravenous injection of 1×10¹¹ vp of Ad5.CMV or Ad.MBP.CMV into adult C57BL/6J mice. Ad5.CMV expression was widespread and robust in liver hepatocytes (top left panel) and punctate within splenic marginal zone (top right panel). Ad.MBP.CMV markedly reduced vector expression in liver hepatocytes (bottom left panel) with increased vector targeting to splenic marginal zone (bottom right panel). Co-staining of spleen sections with EC markers endomucin and CD31 indicated that Ad.MBP.CMV was targeted to mixed ECs and other cell population(s). (B) Immunofluorescence microscopy analysis of EGFP expression in different regions of the brain following intravenous injection of 1×10¹¹ vp of Ad.MBP.CMV into adult C57BL/6J mice. EGFP expression was widespread throughout the vascular network of the cerebrum, hippocampus, medulla, and cerebellum. Magnification: 100', Red: endomucin/CD31, Green: EGFP immunofluorescence. Blue: DAPI.

FIG. 23 illustrates incorporation of MBP into Ad5 drastically increased viral gene expression to vascular beds of multiple host organs. (A) Immunofluorescence microscopy analysis of vector EGFP expression in host organs following intravenous injection of 1×10¹¹ viral particles (vp) of Ad.MBP.CMV into adult C57BL/6J mice revealed prominent transgene expression in lung, heart, kidney, gastrocnemius muscle, pancreas, small and large bowel, and brain. Co-staining of tissue sections with an EC-specific endomucin/CD31 cocktail revealed that EGFP expression was restricted to the vasculature. (B) EGFP fluorescence per μm² of tissue section area (FI, fluorescence intensity) in each organ derived from Ad5.CMV-injected mice (n=4 for all organs) versus that from Ad.MBP.CMV-injected mice (n=10 for liver, spleen, heart, kidney, muscle, small bowel, and brain; n=7 for lung, pancreas, and large bowel). (C) The percentage of vascular EC area expressing EGFP in each organ derived from Ad5.CMV-injected mice (n=4 for alt organs) versus that from Ad.MBP.CMV-injected mice (n=10 for heart, kidney, muscle, small bowel, and brain; n=7 for lung, pancreas, and large bowel). Bar graph is mean +/− standard deviation asterisk: adjusted p<0.05. Magnification: 100×, Red: endomucin/CD31, Green: EGFP immunofluorescence, Blue: DAPI, Li: liver, S: spleen, Lu: lung, H: heart, K: kidney, M: muscle, P: pancreas, SB: small bowel, LB: large bowel, B: brain.

The EC-expression efficiency of the Ad.MBP.CMV versus Ad5.CMV in multiple organs was determined by quantifying the percentage of total tissue EC area expressing EGFP in each organ. Ad.MBP.CMV targeted greater than 62% of blood vessels in regions of the brain (B), 21% in lung (Lu), 26% heart (H), 33% in kidney (K), 38% in muscle (M), 30% in pancreas (P), 16% in small bowel (SB), and 6% in large bowel (LB) (FIG. 23C). Other than liver and spleen, pancreas and small bowel were the only detected organs where Ad5.CMV produced an appreciable but still rare vascular EC expression (FIG. 23C).

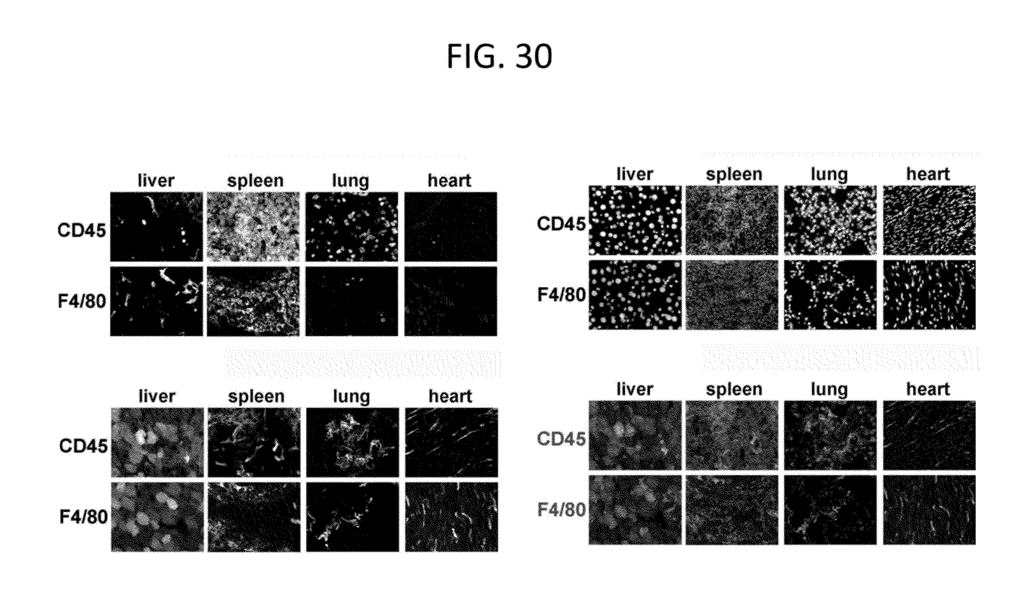

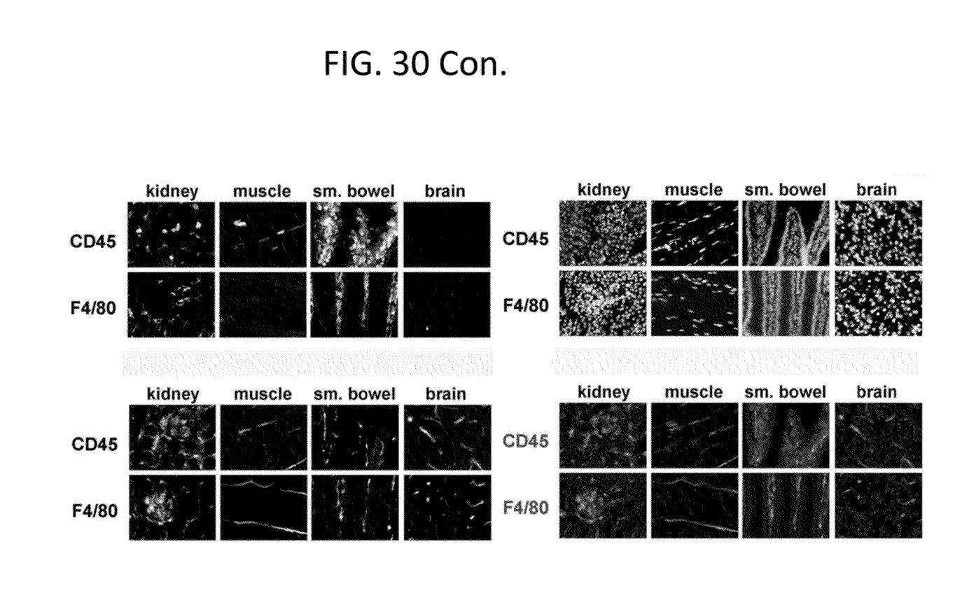

To further test EC-specific expression, immunofluorescence for the pericyte markers. PDGFRβ or proteoglycan nerve-glial antigen 2 (NG2), and the pan-hematopoietic lineage cell marker CD45 or macrophage marker F4/80 were performed. High-magnification revealed that the EGFP-expressing cells were distinct from the PDGERβ-positive or NG2-positive cells in tested organs (FIG. 29). Ad.MBP.CMV was expressed in rare CD45-positive hematopoietic cells and F4/80-positive macrophages in liver and spleen, but not in any other sampled organs (FIG. 30).

FIG. 29 illustrates Ad.MBP.CMV selectively targeted vascular ECs but not pericytes in multiple host organs. High-power magnification EFI (Methods) micrographs of tissue sections co-stained with an endomucin/CD31 cocktail (top panels) and an EGFP antibody localized Ad.MBP.CMV transgene expression to vascular ECs by the in lung, heart, kidney, muscle, small bowel, large bowel, and brain. Tissue sections co-stained for vascular pericyte marker PDGERβ (middle panels) or proteoglycan nerve-glial antigen 2 (NG2, bottom panels) revealed that the EGFP-expressing cells in the organs were distinct from the PDGFRβ+ or NG2+ cells. Magnification: 400×, Red: CD31/endomucin for top-row panels, PDGFRβ for middle-row panels, and NG2 for bottom-row panels. Green: EGFP immunofluorescence, Blue: DAPI.

FIG. 30 illustrates Ad.MBP.CMV targeted cell population(s) distinct from CD45-positive or F4/80-positive cells in most host organs. High-power magnification EFI micrographs of tissue sections co-stained for EGFP and hematopoietic cell marker CD45 or macrophage marker F4/80 in eight-organ panels. The EGFP-expressing cells were distinct from the CD45+ hematopoietic cells and F4/80+ macrophages in lung, heart, kidney, gastrocnemius muscle, small bowel, brain. However, a small fraction of EGFP-positive-cells in liver and spleen expressed CD45 and F4/80. Magnification: 400×. Red: CD45 for top-row panels and F4/80 for bottom-row panels. Green: EGFP immunofluorescence. Blue: DAPI.

### Example 19

This example illustrates that warfarin liver detargeting failed to increase Ad.MBP.CMV multiorgan EC expression.

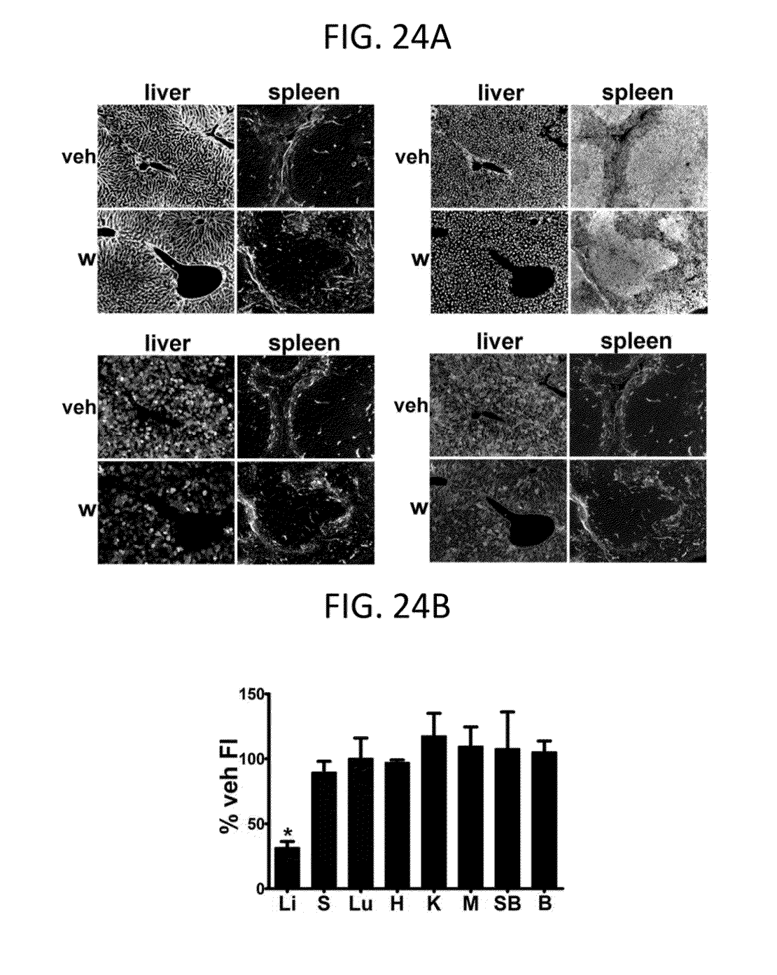

While the Ad.MBP.CMV yielded an impressive level of hepatocyte detargeting, liver remained a substantial transductional and transcriptional target (FIG. 24). As the major pathway directing Ad5 hepalocyte sequestration is mediated by coagulation Factor X-viral hexon binding, it was tested whether warfarin could affect diminution in the level of Ad.MBP.CMV expression in hepatocytes. (Waddington, S. N., et al. 2008) Warfarin pretreatment diminished the number of EGFP-expressing hepatocytes (FIG. 24A). The residual number of vector expressing hepatocytes was similar to our previous work with Ad5 based vectors with wild type capsids. This residual hepatocyte vector expression following warfarin treatment has been seen by others and likely represents a “floor” tor the efficacy of pharmacological blockade. (Waddington. S. N., et al. 2008) However, in contrast to our previous work with vectors with wild type capsids. (Lu, Z. H., et al. 2013) warfarin failed to enhance EC expression in the other testedorgans (FIG. 24A and FIG. 2B). These data suggested that either the Ad.MBP.CMV peripheral vascular EC vector expression was saturated at our 1×10¹¹ viral particle dose, or that complement opsonization facilitated destruction of the vector dose increment that escaped hepatocyte transduction. (Xu. Z., et al. 2013).

FIG. 24 illustrates that warfarin pretreatment reduced Ad.MBP.CMV liver tropism but did not alter gene expression in other host organs. (A) Warfarin, 5 mg/kg, on day −3 and −1 before vector injection diminished hepatocyte expression but did not change transgene expression in spleen. (B) EGFP fluorescence per μm² of tissue area in each organ derived from warfarin-treated mice (n=3 for all organs) normalized as percentage of the mean value of vehicle-treated or untreated counterparts (n=10 for liver, spleen, heart, kidney, muscle, small bowel, and brain; n=7 for lung) with standard deviation. Warfarin pretreatment reduced vector liver expression by 68% (Li) but did not lead to a significant change in gene expression in spleen (S), lung (Lu), heart (H), kidney (K), muscle (M), small bowel (SB), or brain (B). Asterisk indicates adjusted p<0.05. Magnification: 100×, Red: CD31/endomucin, Green: EGFP immunofluorescence, Blue: DAPI.

### Example 20

This example illustrates that Ad.MBP.CMV dose reduction produced organ-specific non-linear EC expression reduction.

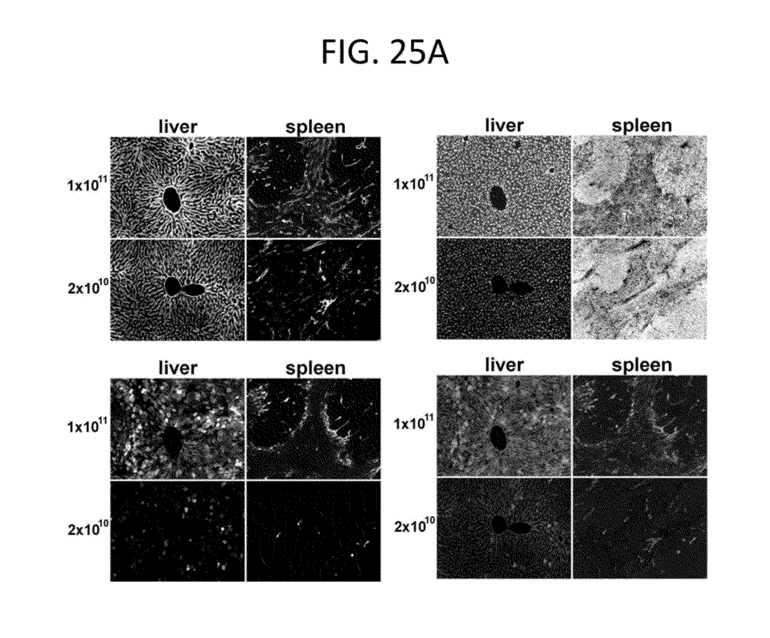

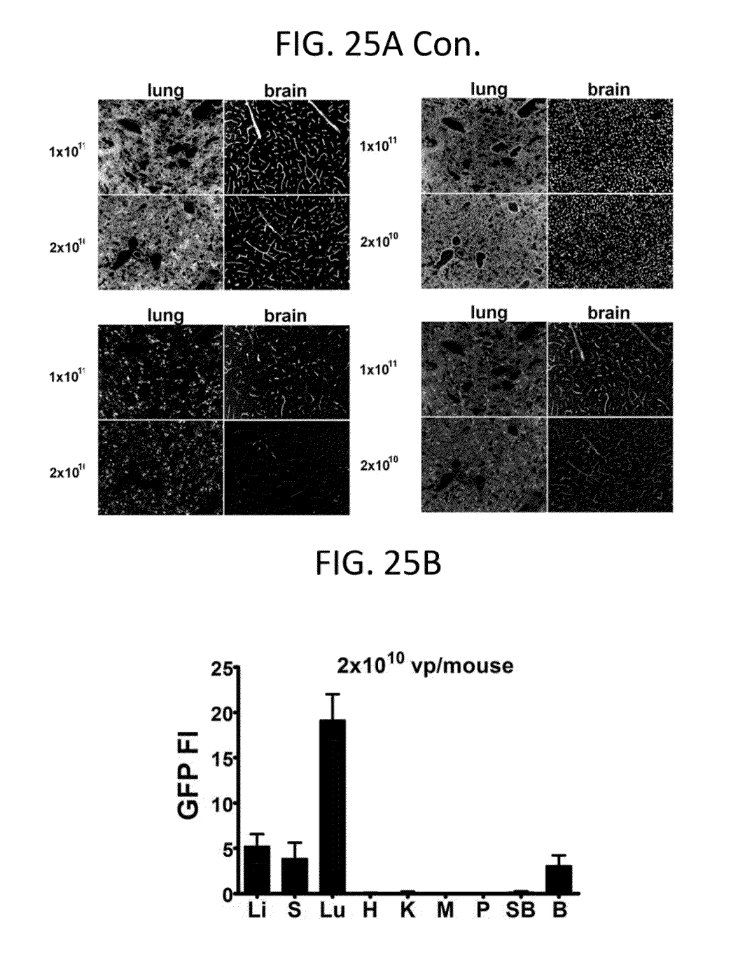

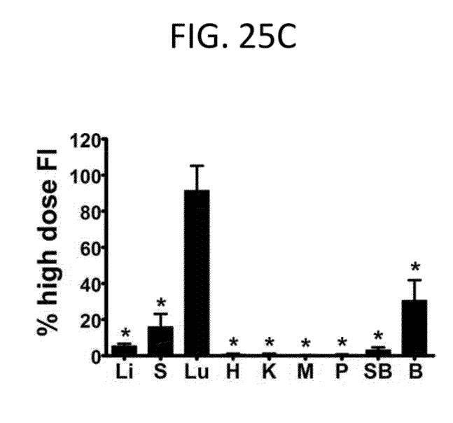

In these experiments, mice were challenged with injection of 2×10¹⁰ viral particles to test the sensitivity of each organ vascular bed for Ad.MBP.CMV expression. The lower viral dose reduced tissue Ad.MBP.CMV expression in liver, spleen, pancreas, heart, kidney, muscle, pancreas, small bowel and brain. Frequency and EC expression level in the lung remained unaffected by vector dose reduction (FIG. 25A and FIG. 25B). Comparison of EGFP fluorescence intensity of low- versus high-dose tissue samples revealed that splenic and brain transgene expression was 16% and 31% of the high-dose counterparts (FIG. 25C, S and B). These levels of reduction in EGFP expression were within the range of linear response to the viral dose difference (20%). However, the diminished expression in liver (5% of high-dose level), heart (0.4% of high-dose level), kidney (0.5% of high dose level), muscle (0.1% of high-dose level), pancreas (0.4% of high-dose level), and small bowel (3% of high-dose level) was nonlinear. Similar to EC expression frequency analysis, vector dose reduction failed to significantly diminish transgene expression in lung (91 % of high-dose level, p=0.588). These results show that Ad.MBP.CMV lung-specific EC expression targeting may be achievable through vector dose fine tuning.

FIG. 25 illustrates systemic administration of a low dose of Ad.MBP.CMV into adult mice produced differential and non-linear reduction in gene expression in host organs. (A) EGFP expression in host liver, spleen, lung, and brain following intravenous injection of 1×10¹¹ or 2×10 ¹⁰ vp of Ad.MBP.CMV into adult mice. Lowering vector dosage significantly reduced EGFP expression in vascular BCs of liver, spleen, and brain but did not change the expression in lung. (B) EGFP fluorescence per μm² of (issue area in each organ derived from the low-dose group (n=6 for each organ). (C) Normalization of the tissue EGFP fluorescence intensity values in (B) to the mean value of the high-dose counterparts. The spleen and brain EGFP expression in low-dose group was 16% and 31% of the high-dose counterparts. However, low virus dose drastically diminished the transgene expression in, heart, kidney, muscle, pancreas, and small bowel (3% of high-dose level). The low dose did not significantly alter the transgene expression in lung (91% of high-dose level). Asterisk indicates p<0.05. Magnification: 100×, Red: endomucin/CD31, Green: EGFP immunofluorescence, Blue: DAP1. Li: liver, S: spleen, Lu: lung, H: heart, K: kidney, M: muscle, P: pancreas, SB: small bowel, B: brain.

### Example 21

This example illustrates that mononuclear cell depletion failed to diminish Ad.MBP.CMV EC transgene expression.

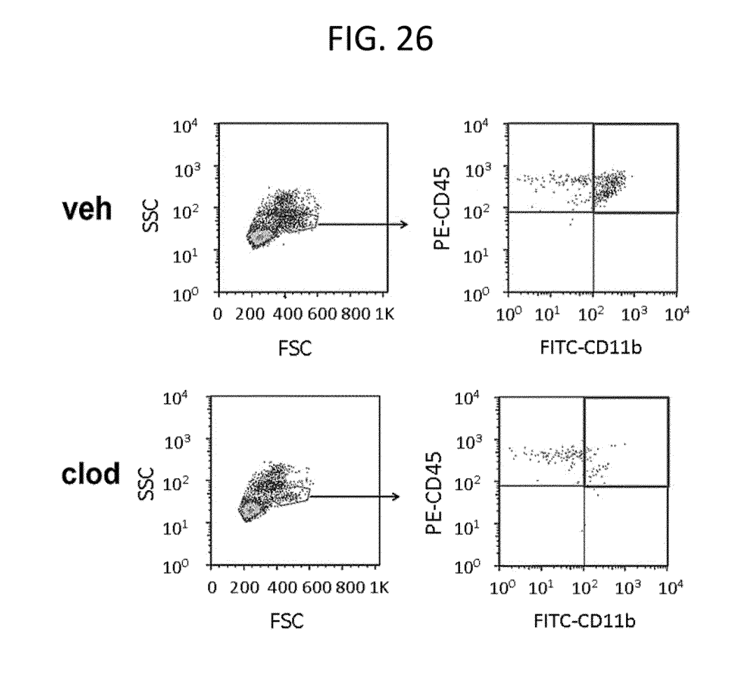

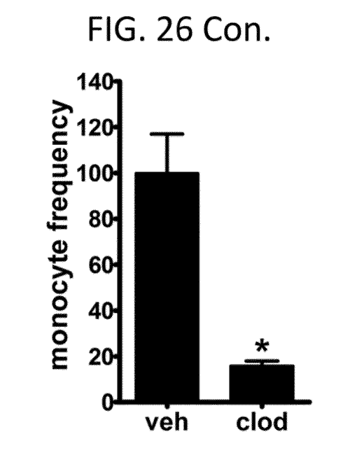

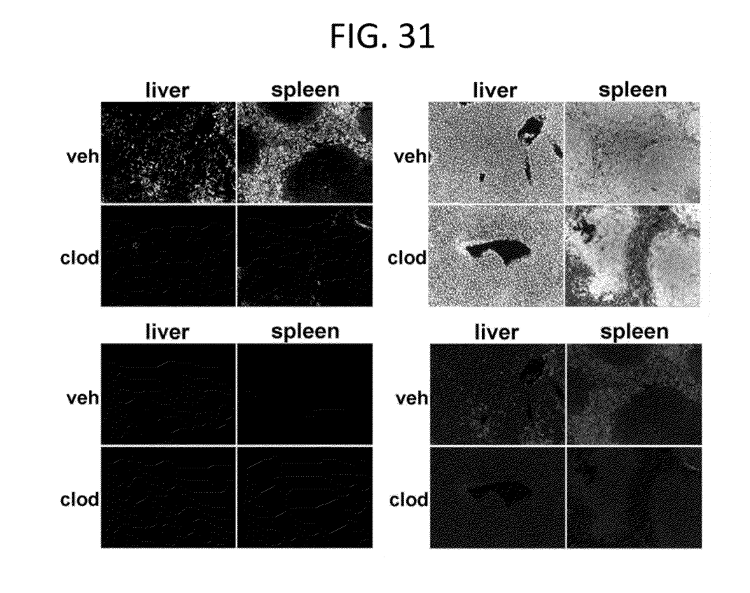

The Ad.MBP.CMV acquired a specific and high affinity binding to myeloid cells ex vivo, compared with the Ad5. (Alberti, M. O. et al. 2013; Alberti, M. O., et al. 2012). In these experiments, two daily doses of intravenous clodronate liposomes were administered to lest the necessity of mononuclear cells for Ad.MBP.CMV EC transgene expression in intact mice. The percentage of circulating CD11b-positive cells and the level of multi-organ tissue section EGFP fluorescence was measured. (FIG. 26). Clodronate treatment depleted circulating CD11b-positive leukocytes by 77% and completely depleted F4/80-positive macrophages fat liver (Kupller cells) and spleen (FIG. 26 and FIG. 31). In contrast, clodronate barely reduced resident macrophages in lung, small bowel, heart, and kidney (data not shown). Clodronate increased Ad.MBP.CMV EC lung expression by 2-fold but did not significantly alter EC transgene expression level or the EC-specific expression pattern in liver, spleen, heart, kidney, muscle, pancreas, small bowel, or brain. The lack of increase in hepatocyte was surprising given prior reports on the scavenging function of liver Kupffer cells (Wolff, G., et al. 1997) however, others have also reported a modest, though statistically insignificant level of clodronate-mediated Ad vector liver expression enhancement. (Bradshaw, A. C., et al. 2012) These data demonstrate that circulating monocytes and macrophages are dispensable for Ad.MBP.CMV organ EC expression. However, the persistence of extrahepatic organ Cd11b and F4/80 cells following clodronate depletion does not rule out the marginated myeloid cell pool as a mechanism for vector-EC handoff. (Alberti, M. O. et al. 2013).

FIG. 26 illustrates depletion of circulating monocytes and hepatic and splenic macrophages lead to an increased Ad.MBP.CMV gene expression in the lung without a significant change in gene expression in other organs. (A) Representative flow cytometry plots (left panel) quantifying the FSC-high/SSC-low/CD11b-positive/CD45-positive monocyte population in circulation. Relative frequency (right panel) of circulating monocytes from elodronate liposome-treated mice (clod. n=3) versus saline-treated mice (veh, n=3). Intravenous injection of elodronate liposomes depleted circulating CD11b-positive cells by 84%. EGFP fluorescence per μm² of tissue area in each organ derived from the saline-injected mice (n=7 for liver, spleen, heart, kidney, muscle, pancreas, small bowel, and brain; n=4 for lung) versus elodronate liposome-injected mice (n=8 for liver, spleen, heart, kidney, pancreas, small bowel, and brain; n=7 for muscle; n=5 for lung). Intravenous elodronate increased Ad.MBP.CMV lung expression by 2-fold but did not result in a significant change in gene expression in liver, spleen, heart, kidney, muscle, pancreas, small bowel, or brain. FIG. 31 illustrates depiction of hepatic and splenic macrophages by elodronate liposomes. Micrographs show P4/80 expression in liver and spleen from saline-treated mice (veh) or elodronate liposome-treated mice (clod). Clodronate-liposome treatment completely depleted F4/80-positive macrophages in liver (Kupffer cells) and in spleen red pulp region. Magnification: 100×, Red: F4/80, blue: DAPI

### Example 22

This example illustrates that EC-specific ROBO4 gene promoter/enhancer ablated the MBP vector hepatocyte expression but reduced EC expression in other organs.

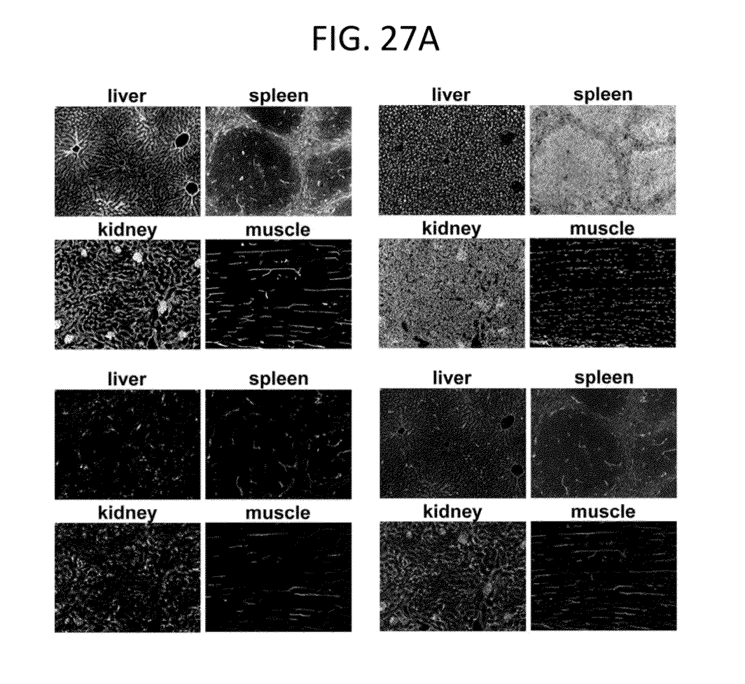

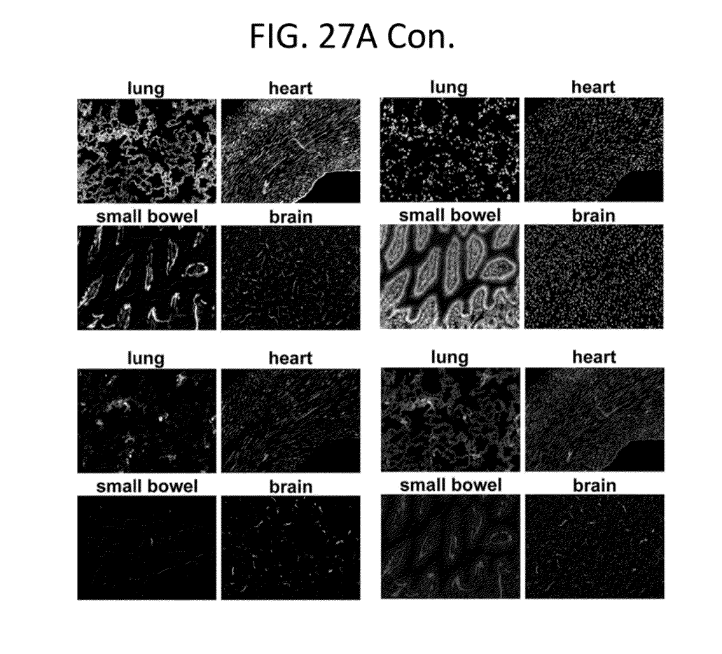

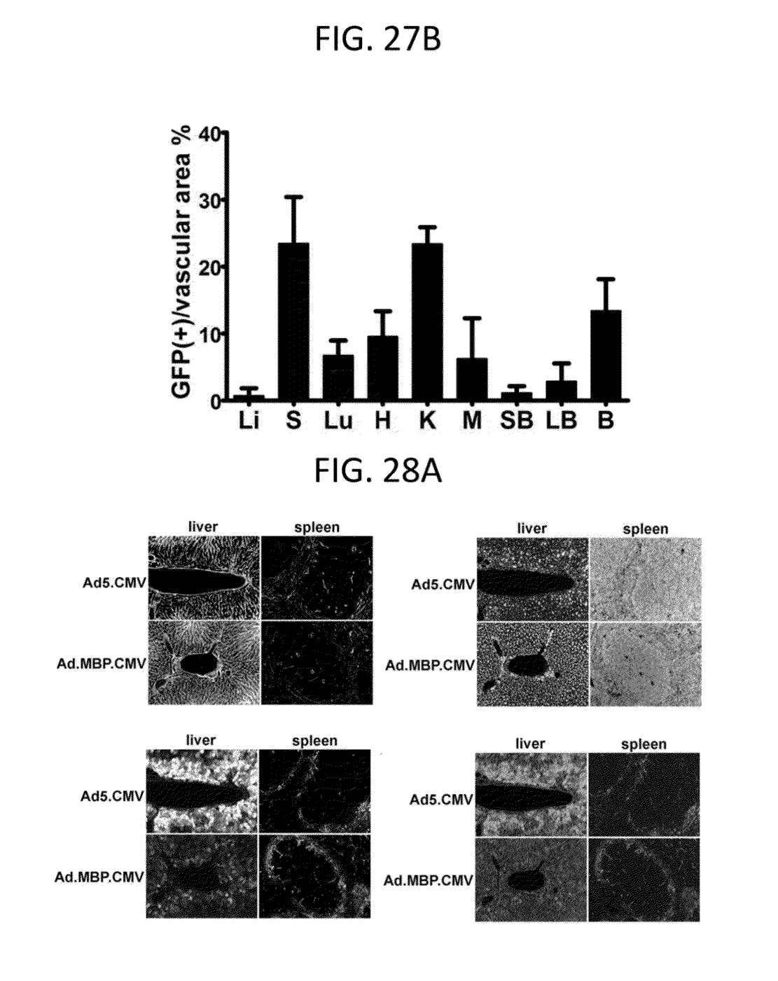

In these experiments, an Ad5 vector was engineered for transcriptional targeting of ECs using the EC-specific human ROBO4 gene enhancer/promoter fragment (Kaliberov, S. A., et al. 2013; Lu. Z., et al. 2013) The CMV promoter was replaced with the ROBO4 enhancer/promoter to test whether the combination of transcriptional with transductional targeting could produce enhanced multi-organ EC expression. The dual targeted Ad.MBP.ROBO4 vector was administered intravenously and organs were analyzed for vector transgene expression (FIG. 27A). Ad.MBP.ROBO4 abrogated hepatocyte expression and instead EGFP was detectable in a scattered population of liver ECs (FIG. 27A). The Ad.MBP.ROBO4 also produced EC transgene expression in an appreciable vascular area fraction in spleen (23%). kidney (23%), lung (10%), muscle (9%), heart (10%), and brain (15%) but produced very low expression in small bowel and large bowel (1% and 2% respectively) (FIG. 27B, S, Lu, H, K, M, SB, LB, and B). Collectively, the ROBO4 enhancer/promoter produced a lower host organ EG expression compared to Ad.MBP.CMV in each organ. However, the undetectable vector transgene expression in hepatocytes highlighted the enhanced endothelial cell type stringency of the ROBO4 compared to the CMV promoter in the Ad.MBP vector.

FIG. 27 illustrates Ad.MBP.ROBO4 detargeted hepatocyte expression but reduced levels of vascular EC expression in other host organs. (A) EGFP expression following intravenous injection of 1×10¹¹ vp of Ad.MBP.ROBO4 into adult mice. Ad.MBP.ROBO4 yielded punctate vascular EC expression in liver but showed a reduced targeting efficiency to vascular ECs in spleen, lung, heart, kidney, muscle, small bowel, and brain. (B) The EGFP-positive vascular area analysis was performed as shown in FIG. 23C. Magnification: 100×, Red: endomucin/CD31, Green; EGFP immunofluorescence, Blue; DAPI, Li: liver, S: spleen, Lu: lung, H: heart. K: kidney. M; muscle, SB: small bowel, LB: large bowel, B: brain.

### Example 23

This example illustrates Ad.MBP.ROBO4-EGFP and Ad.RGD.ROBO4-EGFP expression in Infarct/Reperfusion (I/R) and non-I/R regions.

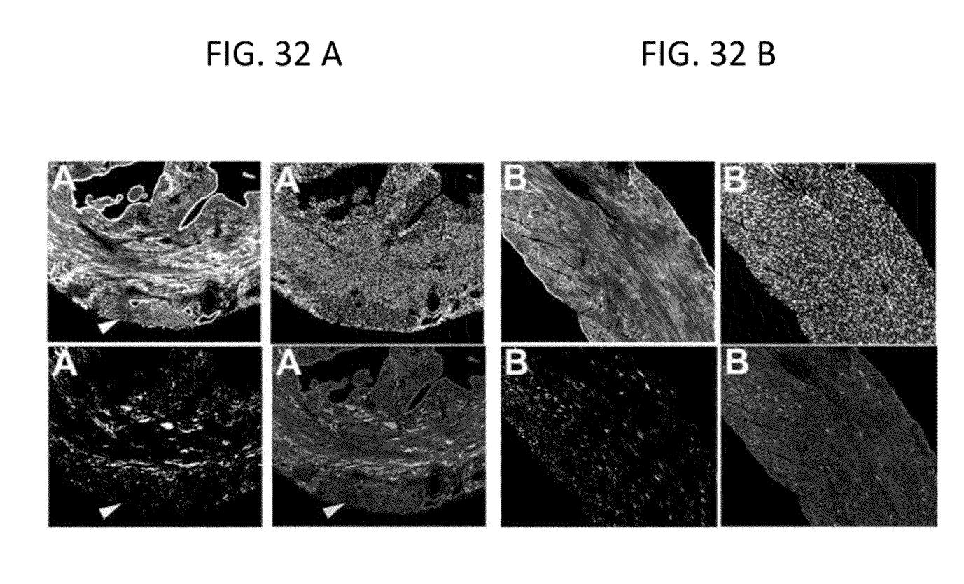

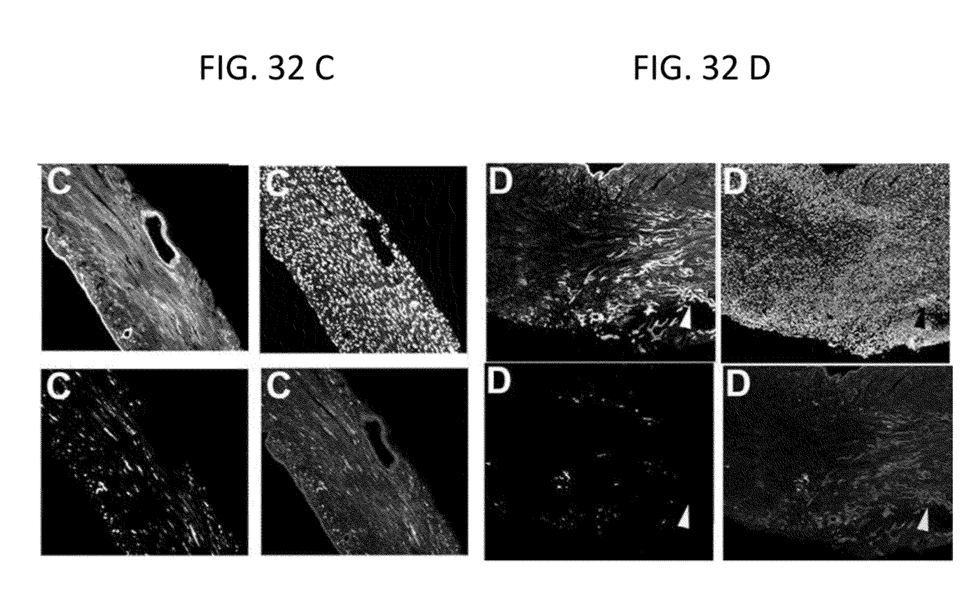

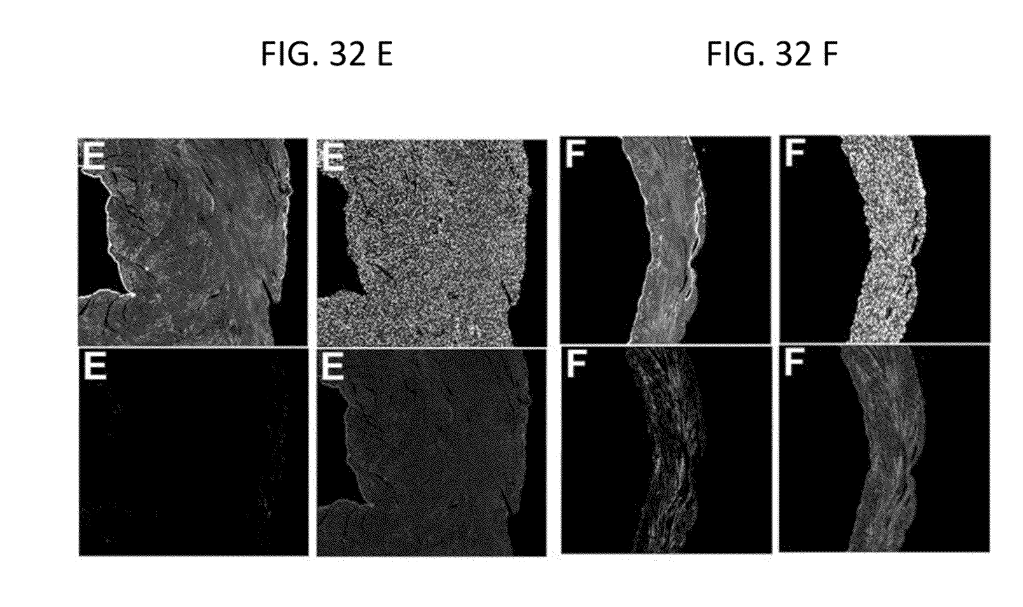

In these experiments, mice were subjected to suture induced left anterior coronary artery ischemia/reperfusion. One day later, either Ad.MBP.ROBO4-EGFP or Ad.RGD.ROBO4-EGFP was injected intravenously. The left ventricle evidenced injury as evidenced by monocyte infiltration (FIG. 32A and data not shown), frank infarction (FIG. 32D), and angiogenesis (arrowheads in FIG. 32A and FIG. 32D). Both vectors were expressed in the I/R region. Ad.MBP.ROBO4-EGFP was induced as indicated by the green EGFP immunofluorescence (FIG. 32A) in the I/R region, whereas it was expressed in multiple vessels in other heart regions not subject to I/R, but at a lower level (FIG. 32B and FIG. 32C). In contrast, Ad.RGD.ROBO4-EGFP expression was restricted to the I/R region (FIG. 32D), albeit at a lower level than Ad.MBP.ROBO4-EGFP. Ad.RGD.ROBO4-EGFP was not expressed in non-I/R regions such as the left ventricular septum or the right ventricular wall (FIG. 32E and FIG. 32F respectively).

### Example 24

This example illustrates uses of the Ad.MBP platform to enhance and/or facilitate limb salvage.

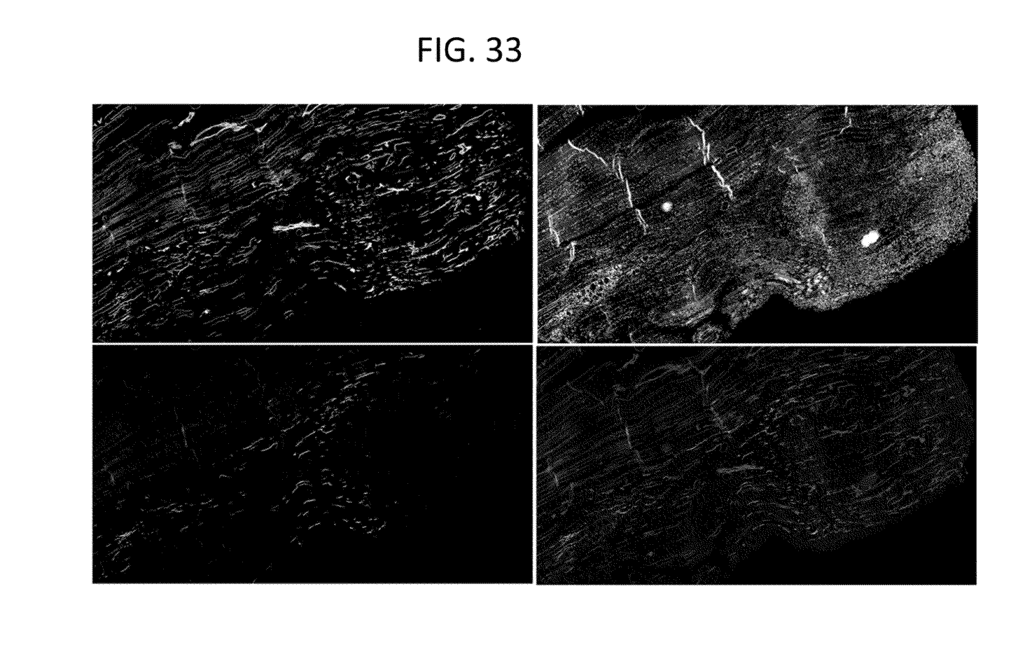

In these experiments, both AdMBP.CMV (Lu et al. 2014) and Ad.MBP.ROBO4 vectors can be induced in the vascular endothelium of the adductor skeletal muscle following hindlimb ischemia secondary to femoral artery ligation in a mouse (FIG. 33). Vectors using an Ad.MBP platform can be loaded with transgene(s)-expressed secreted angiogenic and arteriogenic growth factors and/or transcription factors such as constitutive H1F1-alpha (Oladipupo et al. 2011), H1F2-alpha mutants and/or other master regulatory transcription factors. These factors can have the ability to coordinately induce suites of gene targets mediating a plethora of molecules that can enhance and/or facilitate limb salvage in the context of atherosclerotic disease alone or as a consequence of diabetic vasculopathy.

### Example 25

This example illustrates uses of Ad.MBP vectors to treat conditions activating angiogenesis in villous endothelium.

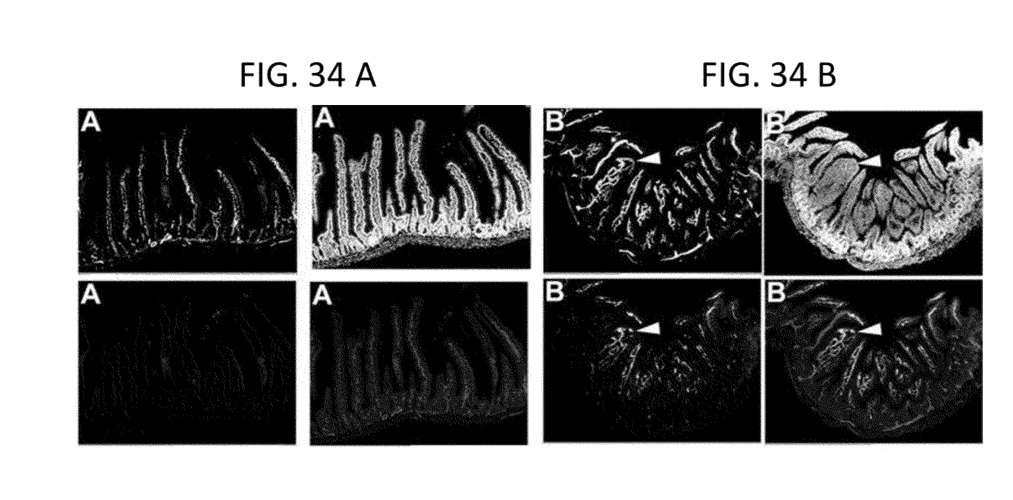

Ad.MBP vectors are expressed in small and large intestinal vascular endothelium (FIG. 34) (Lu et al. 2014). Ad vector Ad.MHP.ROBO4 can be specifically induced in angiogenic intestinal villous vascular endothelial cells following massive small bowel resection, in contrast to a lack of expression in sham-operated small bowel. An Ad.MBP.ROBO4 vector can be specifically expressed in other conditions activating angiogencsis in villous endothelium such as the inflammatory bowel diseases regional enteritis and inflammatory bowel disease of the colon, infections with toxin producing bacteria such as Clostridium difficile, Clostridium botulimim, Shigella, and in the colon cancer precursor lesions of multiple polyposis. Intestinal vascular-trophic vectors can be armed with transgenics that produce secreted anti-inflammatory cytokine decoys such as soluble TNF-alpha receptor, or single chain anti-IL1/IL17 antibodies, bacterial anti-toxins, and RNAi molecules targeting gene products induced by the activation of the VVNT pathway in multiple polyposis.

### Example 26

This example illustrates use of an Ad.MBP.CMV vector to treat inflammatory diseases and degenerative diseases.

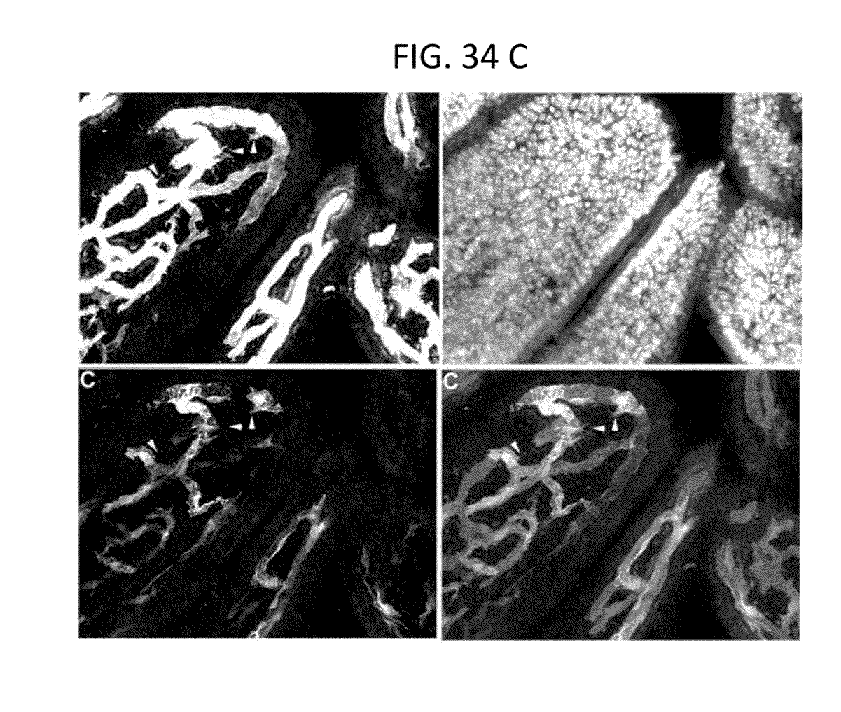

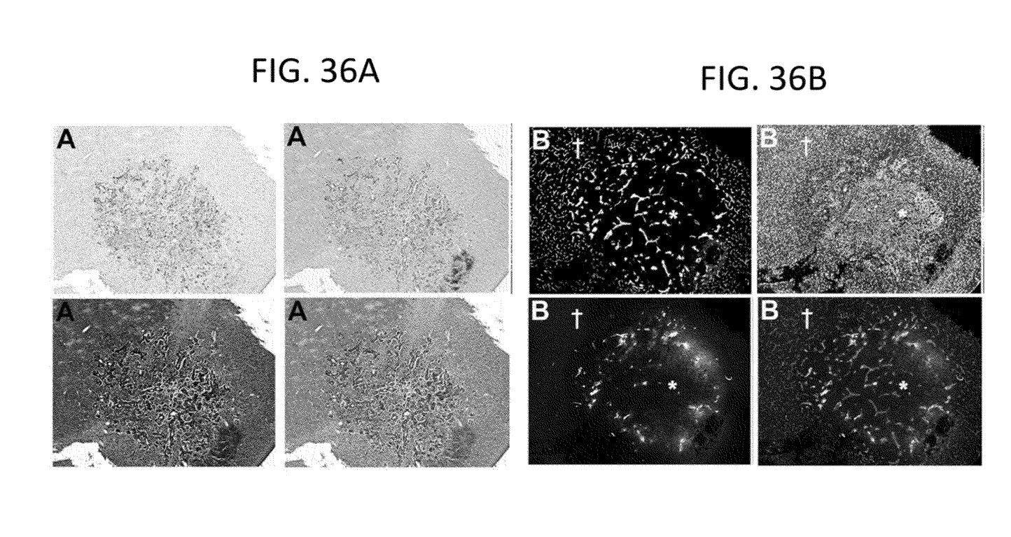

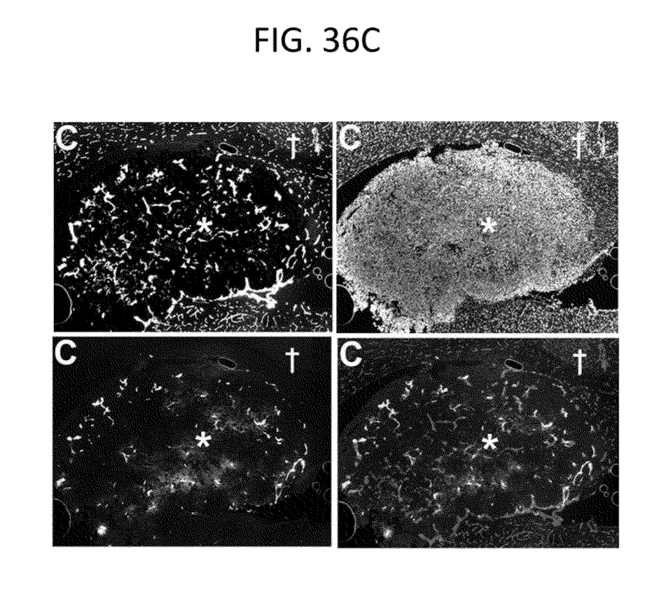

The Ad.MBP.CMV vector can be expressed in all regions of the brain (Lu et al. 2014). This diffuse expression pattern can be used to produce secreted proteins engineered to cross the blood brain barrier and designed to treat inflammatory diseases such as amyotrophic lateral sclerosis and multiple sclerosis and degenerative diseases such as Alzheimer's and Parkinson's. (FIG. 35). For primary and metastatic brain tumors in particular, an Ad.RGD.H5/H3 vector was specifically expressed within the metastatic vasculature but not in normal brain vasculature (FIG. 36B and FIG. 36C). Data also demonstrated expression of the Ad.MBP.CMV vector in the brain vasculature surrounding the hypothalamus (FIG. 35). An Ad.MBP.CMV vector can be engineered to express secreted molecules affecting the hypothalamic appetite nuclei (arcuate). Vectors, such as the vectors in this example, can be used to stimulate appetite in patients suffering from cachexia either due to cancer or benign conditions, or to induce satiety in obese patients with the metabolic syndrome.

### Example 27

This example illustrates use of Ad.RGD.H5/H3.ROBO4 and parental Ad.ROBO4 vectors to treat cancers, produce anti-inflammatory molecules to treat myelodysplastic syndrome bone marrow, and/or correct genetic diseases.

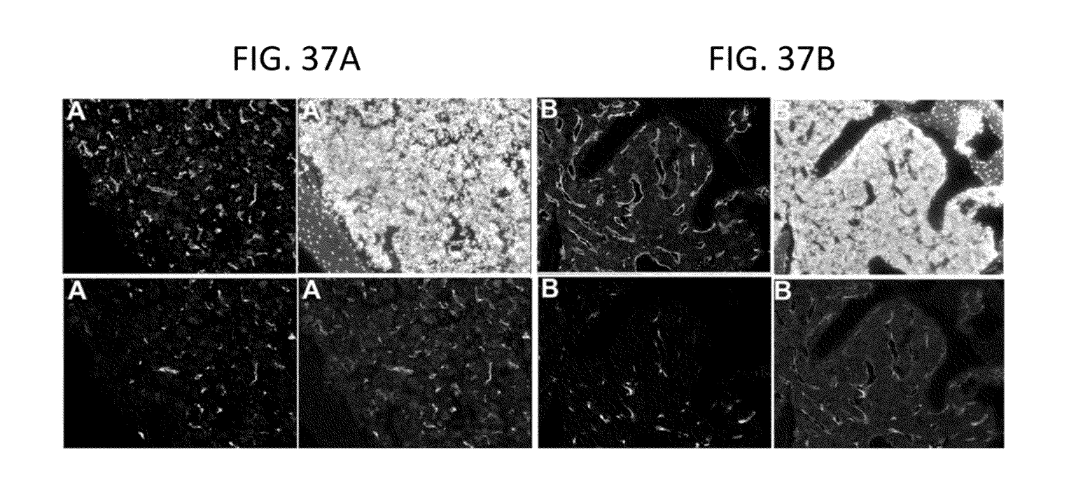

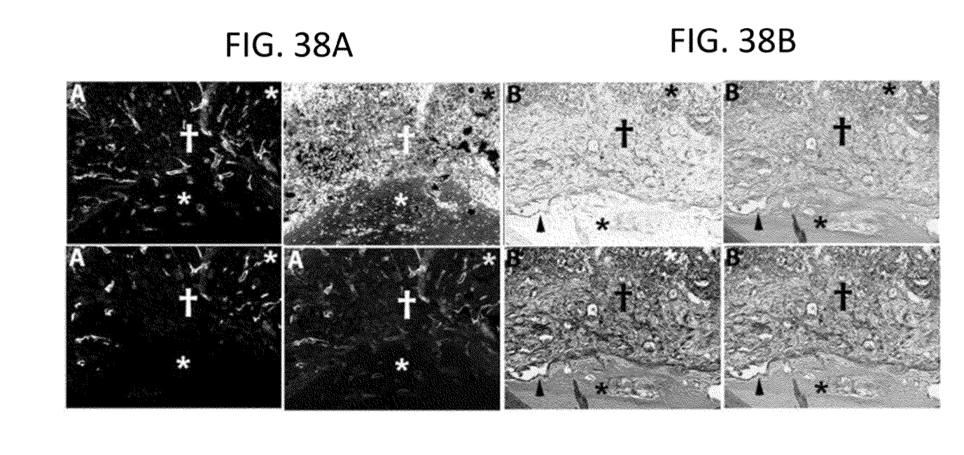

The Ad.RGD.H5/H3.ROBO4 and parental Ad.ROBO4 vectors are expressed throughout the sinusoidal endothelium of the bone marrow (FIG. 37). These vectors can be engineered to express secreted molecules that can mobilize metastatic cancer or leukemic stem cells from their protected niches for chemo-irradiation sensitization (such as molecules described in Nervi et al. 2009), kill metastatic cancers due to chemotherapeutic prodrug converting enzyme production (such as molecules described in Ouyang et al. 2011), to produce anti-inflammatory molecules to treat myelodysplastic syndrome bone marrow, and/or correct genetic diseases such as hemophilia and sickle cell anemia. FIG. 38 demonstrates expression of Ad.RGD.H5/H3.ROBO4 within the vasculature of metastatic human prostate cancer in the femur of a mouse.

### Example 28

This example illustrates that the angiocrine function of endothelial cells can be manipulated using vascular targeted adenoviral vectors.

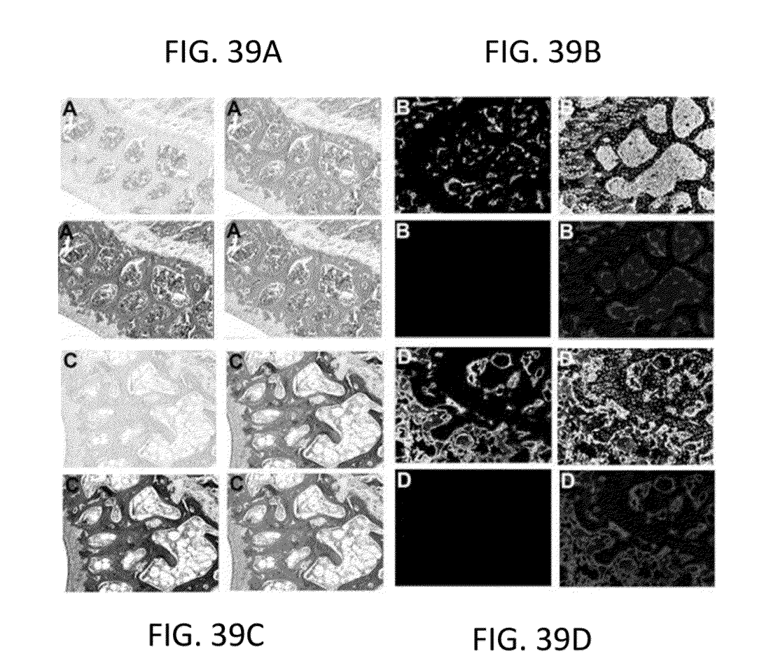

The vascular endothelium can be engineered to secrete molecules that can affect the vascular endothelium's local microenvironments either in tumors or benign diseases. Angiocrine function is the term for the concept of vascular endothelium regulating its microenvironment via molecular secretion. Regarding Ad.ROBO4-bCD (bacterial cytosinc deaminase enzyme), for example, the cytosine deaminase enzyme converts the inactive prodrug 5-fluorocytosine (5-FC) to chemotherapeutic, 5-fluorouracil (5-FU). Mice were warfarinized because this drug prevents liver sequestration of the Ad.ROBO4 vector (not necessary with Ad.RGD.H5/H3 or Ad.MBP vectors FIGS. 32-37 above), and administered 5-FC for 12 days. There was a focal ablation of the bone marrow hematopoietic elements presumably due to 5-FU production and secretion by adjacent vascular endothelial cells. The non-dividing blood vessels were preserved, albeit dilated, despite destruction of the adjacent hematopoietic cells (FIG. 39).

These data demonstrate that the angiocrine function of endothelial cells can be manipulated using vascular targeted adenoviral vectors including vectors listed in this example.

FIGS. 32A-32F illustrate induced expression of Ad.MBP.ROBO4-EGFP and Ad.RGD.ROBO4-EGFP vectors in region of ischemia-reperfusion (I/R) in a suture mouse model. FIG. 32A illustrates Ad.MBP.ROBO4 expression in the left ventricular IR region. FIG. 32B illustrates Ad.MBP.ROBO4 expression in left ventricular septum. FIG. 32C illustrates Ad.MBP.ROBO4 expression in right ventricular free wall FIG. 32D illustrates Ad.RGD.ROBO4 expression in left ventricular VR region. FIG. 32E illustrates Ad.RGD.ROBO4 expression in left ventricular septum. FIG. 32F illustrates Ad.RGD.ROBO4 expression in right ventricular free wall. Red: vascular endothelial specific immunofluorescence using a CD31/endomucin antibody cocktail. Green: EGFP immunofluorescence. Blue: DAPI nuclear stain, Magnification: 40×.

FIG. 33 illustrates Ad.MBP.ROBO4-EGFP expression in the vascular endothelium of the adductor (thigh) muscle following hindlimb ischemia secondary to femoral artery ligation. Red, Green, Blue as in FIG. 32. Mag: 40×.

FIGS. 34A-34C illustrate adenoviral vector expression localized within angiogenic villi in a small bowel resection (SBR) model. FIG. 34A illustrates mice injected with Ad.MBP.ROBO4-EGFP five days post sham surgery. FIG. 34B illustrates endothelial and possible lymphatic expression of the same vector in angiogenic villi post SBR. FIG. 34C illustrates high power view of villous in FIG. 34B (arrowhead) showing colocalized vector transgene expression in angiogenic sprouting endotlielium (arrowheads indicate sprouts). FIG. 34A and FIG. 34B 100×, FIG. 34C 400×.

FIG. 35 illustrates Ad.MBP.CMV vector expression in the vascular endothelium surrounding the hypothalamus (encircled). Red, Green, Blue as in FIG. 32. Mag: 40×.

FIGS. 36A-36C illustrate expression of Ad.RGD.H5/H3 vector within the vascular endothelium of human prostate brain metastases in a mouse. FIG. 36A illustrates a histological section that is adjacent to FIG. 36B. FIG. 36C illustrates a prostate brain metastases in another mouse. Asterisks denote metastases, cross uninvolved brain. Red, Green, Blue as in FIG. 32. Mag: 100×.

FIGS. 37A-37B illustrate Ad.RGD.H5/H3.ROBO4 vector expression in bone marrow sinusoidal endothelium. FIG. 37A illustrates cortical bone marrow in bone shaft. FIG. 37B illustrates trabecular bone marrow near bone end and cartilaginous plate. Red, Green, Blue as in FIG. 32. Mag: 100×.

FIGS. 38A-38B illustrate expression of Ad.RGD.ROBO4-EGFP in a IGR-CaP1 human prostate cancer femoral bone metastases in NOD/SCID/IL2Rγ immunodeficient mouse. FIG. 38A illustrates an adjacent section to FIG. 38B. Green and yellow asterisks (top of picture) are hematopoietic cells adjacent to metastasis. White and black asterisks (bottom of picture) are de novo, osteoblastic bone. While and black crosses are metastatic cells. Arrowhead delineates osteoblastic “rimming”, a pathological hallmark of osteoblastic metastases. Red, Green, Blue as in FIG. 32. Mag: 100×.

FIGS. 39A-39D illustrate angiocrine production of 5-fluorouracil (5-FU) from bone marrow sinusoidal endothelial cells expressing cytosine deaminase (bCD) from an Ad.ROBO4 vector. FIGS. 39A-39D illustrate bone trabecular histology from a mouse injected with Ad.ROBO4-EGFP control virus. FIG. 39B illustrates corresponding vascular marker immunofluoresence. FIG. 39C illustrates bone trabecular histopathology S-FC treated mice following Ad.ROBO4-bCD and preinjection warfarin to detarget liver hepatocyte vector sequestration. FIG. 39D illustrates vascular immunofluorescence demonstrating dilated but intact vasculature and apoptotic hematopoietic cells. Red and Blue as in FIG. 32. Mag: 100×.

All references cited herein are incorporated by reference, each in its entirety. Applicant reserves the right to challenge any conclusions presented by the authors of any reference.

### REFERENCE CITED

Aird, W. C Endothelial biomedicine, Cambridge; N.Y.: Cambridge University Press, 2007, p. x1, 1856 p., 1824 p. of plates.
Al Nakouzi N., et al. Neoplasia 2012;14:376-387.
Alba R., et al. Blood. 2010;116:2656-64.
Alberti M. O., et al. Gene Ther 2012.
Alberti M. O., et al. PLoS One 2012;7:e37812.
Alberti, M. O., et al. Gate therapy 2013;20(7):733-741. Epub 2012 Nov 22.
Assi, H., et al. Neuroscience letters 2012;527(2):71-77,
Azuma M., et al. Biochem Biophys Res Commun 2005;338:1164-1170.
Baehtarzi H., et al. J Control Release. 2011;150:196-203.
Bachtarzi. H., et al. Journal of drug targeting 2011; 19(8):690- 700.
Baker A. M., et al. Exp Physiol. 2005:90:27-31.
Barker N., et al. Cell Stem Cell 2010;7:656-670.
Bedell V. M., et al. Proc Natl Acad Sci U S A. 2005;102:6373-8.
Belousova, N., et al. Journal of Urology 2003:77(21):11367-11377.
Bergers G., et al. Nat Rev Cancer. 2008:8:592-603.
Borghese C. et al. J Cell Biochem 2013:114:1135-1144.
Bradshaw, A. C, et al. Journal of controlled release: official journal of the Controlled Release Society 2012;164(3):394-402.
Bradshaw, A. C, et al. Vascular pharmacology 2013;58(3):174-181.
Brenner A., et al. Clin Cancer Res 2013.
Butler J M. et al. Nat Rev Cancer. 2010;10:138-46.
Carey B W, et al. Proc Natl Acad Sci U S A 2009:106:157-162.
Chang H H, et al. J Biomed Sci 2011;18:6.
Chauchereau A, et al. Exp Cell Res 2011;317:262-275.
Chung L W K. et al. Bone and Cancer. F. Bonner and M. C. Farach-Carson. London. Springer-Verlag. 2009.5: 157-366.
Cole, C, et al. Nature medicine 2005;11(10):1073-1081.
Cook K M, et al. CA Cancer J Clin. 2010:60:222-243.
Coune, P. G., et al. Cold Spring Harbor perspectives in medicine 2012;2(4):a009431.
Crawford Y, et al. Cancer Cell. 2009; 15:21-34.
Ding L. et al. Nature 2013:495:231-235.
Ding L et al. Nature. 2012;481:457-462.
Dong Z, et al Adv. Drug Deliv Rev. 2009;61:542-553.
Duarte S, et al. Cancer Lett 2012:324:160-170.
Dubrovska A, et al. PLoS One 2012;7:e31226.
Duffy M R, et al. Nanomedicine (Lond). 2012;7:271-288.
Economopoulou M, Nat Med 2009;15:553-558.
English H F, et al. Prostate 1987;11:229-242.
Essers M A. et al. Mot Oncol 2010;4:443-450.
Falkowska-Hansen B. et al. Exp Cell Res 2010;316:1885-1895.
Ferrara N. Curr Opin Kematol. 2010;17:219-24.
Ferrara N. Endocr. Rev. 2004;25:581-611.
Ferrer-Vaquer A, et al. BMC Dev Biol 2010:10:121.
Foudi A, et al. Nat Biotechnol 2009;27:84-90.
Fritz V, et al. J Cell Biochem 2011; 112:3234-3245.
Fuchila M, et al. Cancer Res 2009;69:4791 -4799.
Funahasht Y, et al. Cancer Res 2008;68:4727-4735.
Galan-Moya, E. M. et al. EMBO reports 2011;12(5):470-476.
Glinsky V V. Cancer Metastasis Rev 2006;25:531-540.
Goldman C K, et al. Proc Natl Acad Sci USA 1998;95:8795-8800.
Goldstein A S, et al. EMBO Rep 2012:13:1036-1037.
Gordan J D, et al. Cancer Cell. 2008:14:435-46.
Greenbaum A, et al. Nature 2013;495:227-230.
Greenberger S. et al. J Clin Invest 2004:113:1017-1024.
Guo C, et al. Methods Mol Biol 2012;879:315-326.
Hadjantonakis A K. et al. Nat Rev Genet 2003:4:613-625.
Hadjantonakis A K. et al. BMC Biotechnol 2004;4:33.
Haisma H J. et al. Int J Pharm. 2010;391:155-161.
Hatfield M J. et al. Biochem Pharmacol 2011;81:24-31.
He, T. C., et al. Proceedings of the National Academy of Sciences of the United Stales of America 1998;95(5):2509-2514.
He Y. et al. Gene Expr 2010;15:13-25.
Heidenreich R. et al. Cancer Res 2000;60:6142- 6147.
Hirbe A C. et al. Proc Natl Acad Sci U S A 2007;104:14062-14067.
Horvath L G, et al. Prostate 2007;67:1081-1090.
Huminiecki L, et al. Genomics. 2002:79:547-52.
Ibrahim T. et al. Cancer 2010; H6:1406-1418.
Jaggar R T, et al. Hum Gene Ther 1997;8:2239-2247.
Jones C A. et al. Nat Med. 2008;14:448-53.
Jones C A, et al. Nat Cell Biol. 2009; 11:1325-31.
Kalihcrov S A, et al. Cancer Gene Ther 2006; 13:203-214.
Kaliberov. S. A., et al. Virology 2013;447(1-2):312-325.
Kamdar. F., et al. Canadian journal of physiology and pharmacology 2012;90(10);1335-1344.
Kanda T, et al. Curr Biol 1998;8:377-385.
Karhadkar S S, et al. Nature 2004;431:707-712.
Kaminsky, S. M., et al. Human gene therapy 2013:24(11):948-963.
Kawano Y, et al. Br J Cancer 2009; 100:1165-1174.
Khare R. et al. Curr Gene Ther. 2011;11:241-58.
Kiel M J. et al Cell 2005; 121:1109-1121.
Kievit E. et al. Cancer Res 2000;60:6649-6655.
Kim I H, et al. Proc Natl Acad Set US A 200l;98:13282-13287.
Kim K H. et al. Clin Cancer Res 2012.
Kobayashi H, et al. Nat Cell Biol 2010;12:1046-1056.
Koch A W. et al. Developmental Cell. 2011 :20:33-46.
Koh B I, et al. EMBO Rep 2012;13:412-422.
Konclo K, et al. PLoS Biol. 2003:1:E83.
Kostenuik P J. et al. J Bone Miner Res 2009;24:182-195.
Lavergne E, et al. Oncogene 2011;30:423-433.
Leenders W P, et al. Clin Cancer Res. 2004;10:6222-30.
Leong K G, et al. Differentiation 2008;76:699-716.
Li, C, et al. Proceedings of the National Academy of Sciences of the United States of America 2011;108(34):14258-14263.
Lindemann D. et al. Thromb Haemost. 2009; 102:1135-1143.
Logan M. et al. Genesis 2002;33:77-80.
Lu. Z. H. et al. PLoS one 2013;8(12):e83933.
Lu Z. H., et al. Lab Invest 2014; in press.
Lukacs R U. et al. Nat Protoc 2010;5:702-713.
Mahasreshti P J. et al. Clin Cancer Res 2001 ;7:2057-2066.
Marignol L et al. J Gene Med 2009:11; 169-179.
Mariow R, et al. Proc Natl Acad Sci U S A. 2010;107:10520-5.
Mavria G, et al. Gene Ther. 2000;7:368-76.
Mayle A. et al. Cytometry A 2013;83:27-37.
Miki J, et al. Cancer Res 2007;67:3153-3161.
Mognetti B, et al. J Cell Mol Med 2013:17:287-292.
Morrissey C. et al. Clin Exp Metastasis 2008;25:377-388.
Muhammad A K. et al. Clin Pharmacol Ther 2010;88:204-213.
Muro. S., et al. Current vascular pharmacology 2004;2(3):281-299.
Nagasawa T, et al. Trends Immunol 2011;32:315-320.
Nathwani A C, et al. N Engl J Med 2011:365:2357-2365.
Nervi B. et al. Blood 2009;113:6206-6214.
Nettelbeck D M, et al. Mol Ther. 2001;3:882-891.
Nowotsehin S, et al. BMC Dev Biol 2013;l3:15.
Okada Y. et al. Blood. 2008;112:2336-9.
Okada Y. et al. Blood 2008;112:2336-2339.
Okada Y, et al. Circ Res. 2007;100:1712-22.
Oladipupo. S. S., et al. Blood 2011; 117(15):4l42-4153.
Omaisu Y. et al. Immunity 2010:33:387-399.
Ou. D. B., et al. PloS one 2013;8(1):e55233.
Ouyang. L., et al. Bioorg Med Chem 2011;19(12):3750-3756.
Park K W. et al. Dev Biol. 2003;261:251-67.
Peled M. et al. Clin Cancer Res. 2009;15:1664-73.
Preuss M A, et al. Open Gene Ther J. 2008;1:7-11.
Qin J, et al. Cell Stem Cell 2012;10:556-569.
Ramaswamy, S., et al. Neurobiology of disease 2012;48(2):243-254.
Raper S E, et al. Mol Genet Metab 2003;80:148-158.
Reynolds, P. N. et al. Nature biotechnology 2001; 19(9):838-842.
Reynolds P N. et al. Mol Ther. 2000;2:562-578.
Rhim J S. Methods Mol Biol 2013;946:383-393.
Rieger M A, et al. Cold Spring Harb Perspect Biol 2012:4.
Rivory L P. et al. Biochem Pharmacol 1996;52:1103-1111.
Roth, J. C. et al. Gene therapy 2008; 15(10):716-729.
Savontaus M J, et al. Gene Ther. 2002;9:972-9.
Schulze J. et al. Bone 2010;46:524-533.
Schulze J, et al. Cancer Lett 2012;317:106-113.
Schweizer L, et al. BMC Cell Biol 2008;9:4.
Seandel M, et al. Proc Natl Acad Sci U S A 2008;105:19288-19293.
Seruga B, et al. J Clin Oncol 2011;29:3686-3694.
Seth P. et al. Biochem Biophys Res Cornmun. 2005;332:533-41.
Shao L, et al. Cell Res 2009;19:296-306.
Sheldon H, et al. FASEB 3.2009;23:513-22.
Shindoh H, et al. J Toxicol Sci 2011 ;36:411-422.
Shinkai Y, et al. Cell. 1992;68:855-67.
Shinozaki K, et al. Gene Ther. 2006;13:52-59.
Shiozawa Y, et al. J Clin Invest 2011; 121:1298-1312.
Short J J, et al. Mol Cancer Ther 2010;9:2536-2544.
Smith-Berdan S, et al. Cell Stem Cell. 2011;8:72-83.
Song W, et al. Cancer Gene Ther. 2008;15:667-75.
Sottnik J L, et al. Cancer Microenviron 2011 ;4:283-297.
Stadtfeld M, et al. Science 2008;322:945-949.
Su X, et al. J Clin Invest 2012; 122:3579-3592.
Sun Y X, et al J Bone Miner Res 2005;20:318-329.
Szymczak A L. et al. Nat Biotechnol 2004:22:589-594.
Szyrnczak-Workman A L, et al. Cold Spring Harb Protoc 2012;2012:199-204.
Takayama K, et al. Cancer Gene Ther 2007:14:105-116.
Takebe N, et al. Nat Rev Clin Oncol 2011;8:97-106.
Tallone T, et al. Proc Natl Acad Sci U S A. 2001;98:7910-7915.
Tang, T., H et al. Translational research: the journal of laboratory and clinical medicine 2013;161(4):313-320.
Topf N. et al. Gene Ther 1998;5:507-513.
Triozzi P L, et al. Expert Opin Biol Ther 2011; 11:1669-1676.
Valdez J M, et al. Cell Stem Cell 2012;11:676-688.
van Berlo. J. H., et al. The Journal of clinical investigation 2013;123(1):37-45.
van Rooijen, N. et al. Methods in molecular biology 2010:605:189-203.
Varda-Bloom N. et al. Gene Ther. 2001;8:819-27.
Voog J, et al. Cell Stem Cell 2010;6; 103-115.
Waddington S N, et al. Cell 2008:132:397-409.
Wang Z. et al. J Cell Biochem 2010:11)9:726-736.
Westerink W M, et al. Mutal Res 2010;696:21-40.
White. K. M., et al. Journal of cardiothoracic surgery 2013;8(1): 183.
Wilson A. et al. Cell 2008:135:1118-1129.
Wolff, G., et al. Journal of Virology 1997;71 (1):624-629.
Xiong W. et al. J Virol 2006;80:27-37.
Xu, Z., et al. Nature medicine 2013; 19(4):452-457.
Yang C. et al. Cancer Discov 2013:3:212-223.
Yang W Y. et al. World J Gastroenterol 2006;12:5331-5.
Ye H, et al. Cancer Invest 2012:30:513-518.
Ye Q-F, et al. Asian Pacific Journal of Cancer Prevention 2012;13:2485-2489.
Zeiss. A. K., et al. Journal of cellular biochemistry 2009;108(4):778-790.
Zeng L., et al. Proc Natl Acad Sci USA 2009:106:8326-8331.
Zeng Q, et al. Int J Nanomedicine 2012:7:985-997.
Zhang X. et al. PLoS Pathog. 20I2;8:el002461.
Zhu T S. et al. Cancer Res. 2011:71:6061-6072.

1. An adenovirus vector comprising a ROBO4 enhancer/promoter operatively linked to a transgene.

2. An adenovirus vector in accordance with claim 1, wherein the transgene encodes a prodrug converting enzyme.

3. An adenovirus vector in accordance with claim 2, wherein the prodrug converting enzyme is a cytosine deaminase.

4. An adenovirus vector in accordance with claim 1, wherein the transgene encodes a decoy receptor.

5. An adenovirus vector in accordance with claim 4, wherein the decoy receptor binds at least one angiocrine factor.

6. An adenovirus vector in accordance with claim 1, wherein the Transgene encodes a truncated CXCR4 receptor.

7. An adenovirus vector in accordance with claim 1, wherein the ROBO4 enhancer/promoter comprises a tissue-specific expression control element.

8. An adenovirus vector in accordance with claim 1, wherein the ROBO4 enhancer/promoter comprises a Tet response element.

9. An adenovirus vector in accordance with claim 1, wherein the ROBO4 enhancer/promoter comprises a hypoxia response element.

10. An adenovirus vector in accordance with claim 1, wherein the ROBO4 enhancer/promoter comprises a GASP-binding element.

11. An adenovirus vector in accordance with claim 1, further comprising:
a chimeric AD5-T4 phage fibritin shaft; and
a trimerization domain displaying a myeloid cell-binding peptide (MBP).

12. A method of expressing a transgene in an endothelial cell in vivo, the method comprising administering to a mammal an adenovirus in accordance with claim 1.

13. A method of mobilizing cells in vivo, comprising administering to a mammal an adenovirus in accordance with claim 6.

14. A method of mobilizing cells in vivo in accordance with claim 13, wherein the cells comprise at least one of granulocytes, monocytes and lymphocytes from bone marrow.

15. A method of mobilizing cells in vivo in accordance with claim 13, wherein the ceils are cancer cells.

16. A method in accordance with claim 15, wherein the cancer cells are comprised by bone marrow.

17. A method of selectively targeting endothelial cells, comprising administering to a mammal an adenovirus comprising a chimeric AD5-T4 phage fibritin shaft and trimerization domain displaying a myeloid cell-binding peptide (MBP), and an exogenous promoter operatively linked to a transgene.

18. A method of selectively targeting endothelial cells in accordance with claim 11, wherein the promoter is a ROBO4 enhancer/promoter.

19. A method of selectively targeting endothelial ceils in accordance with claim 17, wherein the promoter comprises a Tel-responsive element.

20. A method of selectively targeting endothelial cells in accordance with claim 17, wherein the promoter comprises a hypoxia-responsive element.

21. A method in accordance with claim 17, wherein the endothelial cells are selected from the group consisting of brain ECs, kidney ECs and muscle ECs.

22. A method in accordance with claim 17, wherein the transgene encodes a truncated CXCR4 receptor.

23. A method of treating a cancer in a mammal in need thereof, comprising:
administering to the mammal an Ad.RGD.HS/H3.ROBO4 vector,
wherein the Ad.RGD.HS/H3.ROBO4 vector produces at least one molecule selected from the group consisting of a molecule that mobilizes metastatic cancer or leukemic stem cells and a molecule producing a chemotherapeutic prodrug converting enzyme.
